(19) United States
(12) Patent Application Publication
(10) Pub. No.: US 2025/0268951 A1
(43) Pub. Date: Aug. 28, 2025

(54) THERAPEUTIC AND PROPHYLACTIC USE OF MICROORGANISMS

(71) Applicant: THE CHINESE UNIVERSITY OF HONG KONG, Shatin, N.T. Hong Kong SAR (CN)

(72) Inventors: Siew Chien NG, Hong Kong SAR (CN); Ka Leung Francis CHAN, Hong Kong SAR (CN)

(21) Appl. No.: 17/417,732

(22) PCT Filed: Dec. 27, 2019
(86) PCT No.: PCT/CN2019/129092

§ 371 Date: Jun. 23, 2021

Related U.S. Application Data
(60) Provisional application No. 62/785,640, filed on Dec. 27, 2018.

(51) Int. Cl. A61K 35/74; A61K 35/00; A61P 3/04; C12Q 1/04

(52) U.S. Cl. CPC A61K 35/74; A61P 3/04; C12Q 1/04; A61K 2035/115

(57) ABSTRACT

Certain microorganism species in the gut and feces of fecal microbiota transplantation (FMT) donors can affect the outcome of FMT treatment. Thus, novel methods are provided for identifying subjects as suitable donor to optimize FMT outcome and for pre-treating donors and/or receipients for optimized FMT outcome. Also provided are kits and compositions for improving FMT outcome, including for specific purposes such as for weight loss or cholesterol reduction.

### RELATED APPLICATION

This application is the US National Stage Entry under § 371 of International Application No. PCT/CN2019/129092, filed Dec. 27, 2019, which claims priority to U.S. Provisional Patent Application No. 62/785,640, filed Dec. 27, 2018, the contents of which are hereby incorporated by reference in the entirety for all purposes.

### BACKGROUND OF THE INVENTION

Fecal microbiota transplantation (FMT) is a highly effective method for treating a variety of conditions including gastrointestinal disorders and Clostridium difficile infection (CDI), especially among patients suffering from recurring CDI. Also known as stool transplant, FMT involves a process of transplanting fecal matter containing microorganism from a healthy individual into the gastrointestinal tract of a recipient. The goal of FMT is restoration of the gut microflora disrupted due to a disorder such as CDI by introducing (or re-introducing) healthy bacterial flora via various means of infusion of a healthy individual's stool, e.g., by colonoscopy, enema, orogastric tube, or by mouth in the form of a capsule containing freeze-dried material obtained from a healthy donor. Aside from CDI, FMT is increasingly being used to treat other intestinal and extra-intestinal diseases, including other gastrointestinal diseases such as inflammatory bowel disease (IBD), antibiotic-resistant bacterial infection, diarrhea, constipation, irritable bowel syndrome, autism, depression, obesity, diabetes, alopecia, and the like. In addition, FMT has been used for treating certain neurological conditions, such as multiple sclerosis and Parkinson's Disease. Recent studies have revealed that the presence of certain species of microorganisms in the donor material for use in FMT, such as certain species of bacteria and fungi, can significantly impact the efficacy of FMT as well as directly affect whether additional health benefits relevant to metabolism regulation may be conferred to FMT recipients. Considering the prevalence of various conditions and diseases that are treatable by FMT in the human population and their significant economic implications, there exists an urgent need for developing new and improved methods for enhancing FMT therapeutic efficacy and for optimizing treatment outcome, especially on a donor-and/or recipient-specific basis. The present invention fulfills this and other related needs.

### BRIEF SUMMARY OF THE INVENTION

The invention relates to novel methods and compositions useful for optimizing fecal microbiota transplantation (FMT) treatment, especially for maximizing health benefits conferred to recipients of FMT. In particular, the present inventor discovered that, when certain microorganism species (e.g., bacteria, fungi, viruses) are present, especially at an elevated level, in a transplant material for FMT recipient and subsequently in the gastrointestinal (GI) tract of a recipient after receiving FMT treatment, significant health benefits such as weight loss, higher insulin sensitivity, lower blood cholesterol, reduced presence of bacteria with multidrug resistance, alleviated undesirable conditions following hematopoietic stem cell transplant such as acute graft-versus-host disease (aGvHD), and alleviated GI tract conditions such as ulcerative colitis (UC) or inflammatory bowel disease (IBD) or Crohn's disease can be achieved in the FMT recipient; whereas the presence (especially at a higher level) of certain other microorganism species in an FMT recipient GI tract correlates with the lack or greatly diminished health benefits of this nature or essentially ineffective treatment outcome. These findings allow the inventors to devise methods and compositions that can improve FMT efficacy in regard to these health benefits. Thus, the present invention provides a novel method for identifying a suitable donor for FMT, who provides fecal material to be used in FMT after proper processing. The method comprising the step of determining the level of one or more bacterial, fungal, or viral species set forth in Table 1a, 1b, 2, 3a, 3b, 4, 5, 6a, 6b, 7a, 7b, 8a, 8b, 10a, 10b, 11, 15, or 16 in a stool sample obtained from a candidate for FMT donor.

In the first aspect, the present invention provides method for identifying a suitable donor for FMT, comprising the step of determining level of one or more bacterial species set forth in Table 1a, 4, 6a, 10b, 11, or 15 in a stool sample obtained from a candidate. In some embodiments, the level of the one or more bacterial species is a percentage relative abundance. In some embodiments, when the level of the one or more bacterial species is found to be greater than 0.1%, the candidate is identified as a suitable donor for FMT. In some embodiments, the level of the one or more bacterial species set forth in Table 1b, 6b, or 10a is no greater than 0.01%. In some embodiments, the method further comprises a step of obtaining stool material from the suitable candidate for use in FMT. In some embodiments, the method further includes a step of determining total bacterial load in the stool sample. In some embodiments, when the level of the one or more bacterial species set forth in 1b, 6b, or 10a is greater than 0.01%, the candidate is identified as an unsuitable donor for FMT.

In some embodiments, the level of one or more bacterial species set forth in Table 1a, 1b, 4, 6a, 6b, 10a, 10b, 11, 15, or 16 is determined in a first stool sample obtained from a first candidate and in a second stool sample obtained from a second candidate. In some embodiments, the first candidate has a higher level of the one or more bacterial species set forth in Table 1a, 4, 6a, 10b, 11, or 15 than the second candidate and is deemed to be a more suitable FMT donor than the second candidate. In some embodiments, the first candidate has a lower level of the one or more bacterial species set forth in Table 1b, 6b, or 10a than the second candidate and is deemed to be a more suitable FMT donor that the second candidate.

In one example, when a donor is screened for his potential as a suitable FMT donor, especially for the purposes of helping a recipient lose weight, the presence of beneficial bacteria such as Bifidobacterium bifidum, Roseburia intestinalis, and Sutterella wadsworthensis, in his stool sample should reach or surpass a threshold in the relative amount of: e.g., at least about 0.3% or at least 0.7% for Bifidobacterium bifidum, at least about 3.5% or at least about 6% for Roseburia intestinalis, or with at least about 1% of Sutterella wadsworthensis. Conversely, the presence of detrimental bacteria, such as Blautia hydrogenotrophica or a Peptostreptococcaceae bacterium, is preferred to be as low as possible (e.g., less than about 0.001%) or even undetectable. On the other hand, if a candidate donor has in his stool sample inadquate amount of the beneficial bacteria (e.g., less that about 0.1% of each of Bifidobacterium bifidum, Roseburia intestinalis, and Sutterella wadsworthensis) but too much detrimental bacteria (e.g., at least about 0.01%, at least about 0.05%, or at least about 0.07% of Blautia hydrogenotrophica or a Peptostreptococcaceae bacterium), then he should be excluded to serve as a donor, especially when weight reduction is an objective in the proposed FMT process.

In a related aspect, a method is provided for improving FMT efficacy, the method comprising introducing an effective amount of one or more bacterial species set forth in Table 1a, 4, 6a, 10b, 11, or 15 into a composition intended for use in transplantation prior to FMT. In some embodiments, after the introducing step the level of each of the one or more bacterial species set forth in Table 1a, 4, 6a, 10b, 11, or 15 is greater than 0.1% of total bacteria in the composition. In some embodiments, the method further comprises a step of performing FMT using the composition. In some embodiments, the method further comprises a step of introducing into the composition an effective amount of an anti-bacterial agent that suppresses growth of one or more bacterial species set forth in Table 1b, 6b, or 10a. In some embodiments, after the introducing step the level of each of the one or more bacterial species set forth in Table 1b, 6b, 10a is less than 0.01% of total bacteria in the composition. In some embodiments, the method further comprises a step of performing FMT using the composition.

In another related aspect, a method is provided for improving FMT efficacy by administering to an FMT recipient prior to FMT an effective amount of an anti-bacterial agent that suppresses growth of one or more bacterial species set forth in Table 1b, 6b, or 10a. In some embodiments, the level of the one or more bacterial species set forth in Table 1b, 6b, or 10a is determined in a stool sample from the FMT recipient prior to administration of the anti-bacterial agent. In some embodiments, the level of the one or more bacterial species set forth in Table 1b, 6b, or 10a is determined in a stool sample from the FMT recipient after administration of the anti-bacterial agent. In some embodiments, the method further comprises a step of administering to the recipient prior to FMT an effective amount of an agent that reduces total bacterial load in a stool sample taken from the recipient prior to FMT.

In yet another related aspect, a kit is provided that comprises (1) a first composition comprising donor stool; and (2) a second composition comprising (i) an effective amount of one or more bacterial species set forth in Table 1a, 4, 6a, 10b, 11, 15, or 16; or (ii) an effective amount of an anti-bacterial agent that suppresses growth of an anti-bacterial agent that suppresses growth of the one or more bacterial species set forth in Table 1b, 6b, or 10a. In some embodiments, the first composition comprises donor stool that has been dried, frozen, and placed in a capsule for oral ingestion. In some embodiments, the kit further comprises, in the second composition an effective amount of an anti-bacterial agent that suppresses growth of the one or more bacterial species set forth in Table 1b, 6b, or 10a, or in a third composition an effective amount of an anti-bacterial agent that reduces total bacterial load. In addition, the kit may comprise printed instructions to guide the user to properly use the kit.

In some embodiments, in the above described methods the level of the one or more bacterial species set forth in Table 1a, 1b, 4, 6a, 6b, 10a, 10b, 11, or 15 is determined by quantitative polymerase chain reaction (PCR).

In a second aspect, the present invention provides a method for identifying a suitable donor for FMT. The method comprises the step of determining level of one or more fungal specie set forth in Table 2, 3a, 5, 7a, or 16 in a stool sample obtained from a candidate. In some embodiments, the level of the one or more fungal species is a percentage relative abundance. In some embodiments, the level of the one or more fungal species set forth in Table 2, 3a, 5, 7a, or 16 is greater than 0.5% and the candidate is identified as a suitable donor for FMT. In some embodiments, the level of the one or more fungal species set forth in Table 3b or 7b is no greater than 0.05%. In some embodiments, the method further comprises a step of obtaining stool material from a suitable candidate for use in FMT. In some embodiments, the level of the one or more fungal species set forth in Table 3b or 7b is greater than 0.05% and the candidate is identified as an unsuitable donor for FMT. In some embodiments, the method further comprises a step of determining total fungal load in the stool sample.

In some embodiments, the level of the one or more fungal specie set forth in Table 2, 3a, 3b, 5, 7a, 7b, or 16 is determined in a first stool sample obtained from a first candidate and in a second stool sample obtained from a second candidate. In some embodiments, the first candidate has a higher level of the one or more fungal species set forth in Table 2, 3a, 5, 7a, or 16 than the second candidate and is deemed to be a more suitable FMT donor than the second candidate. In some embodiments, the first candidate has a lower level of the one or more fungal species set forth in Table 3b or 7b than the second candidate and is deemed to be a more suitable FMT donor that the second candidate.

In one related aspect, a method is provided for improving FMT efficacy, the method comprising introducing an effective amount of the one or more fungal species set forth in Table 2, 3a, 5, 7a, or 16 into a composition intended for use in transplantation prior to FMT. In some embodiments, after the introducing step the level of the one or more fungal species set forth in Table 2, 3a, 5, 7a, or 16 is greater than 0.5% of total fungi in the composition. In some embodiments, the method further comprises a step of performing FMT using the composition. In some embodiments, the method further comprises the step of introducing into the composition an effective amount of an anti-fungal agent that suppresses growth of one or more fungal species set forth in Table 3b or 7b. In some embodiments, after the introducing step the level of the level of the one or more fungal species set forth in Table 3b or 7b is less than 0.05% of total fungi in the composition. In some embodiments, the method further comprises a step fo performing FMT using the composition.

In another related aspect, a method is provided for improving FMT efficacy, the method comprising administering to an FMT recipient prior to FMT an effective amount of an anti-fungal agent that suppresses growth of one or more fungal species set forth in Table 3b or 7b. In some embodiments, the level of the one or more fungal species set forth in Table 3b or 7b is determined in a stool sample from the FMT recipient prior to administration of the anti-fungal agent. In some embodiments, the level of the one or more fungal species set forth in Table 3b or 7b is determined in a stool sample from the FMT recipient after administration of the anti-fungal agent. In some embodiments, the method further comprises a step of administering to the recipient prior to FMT an effective amount of an agent that reduces total fungal load in a stool sample taken from the recipient prior to FMT.

In yet another related aspect, a kit is provided which comprises (1) a first composition comprising donor stool; and (2) a second composition comprising (i) an effective amount of one or more fungal species set forth in Table 2, 3a, 5, 7a, or 16; or (ii) an effective amount of an anti-fungal agent that suppresses growth of one or more fungal species set forth in Table 3b or 7b. In some embodiments, the first composition comprises donor stool that has been dried, frozen, and placed in a capsule for oral ingestion. In some embodiments, the kit further comprises, in the second composition an effective amount of an anti-fungal agent that suppresses growth of one or more fungal species set forth in Table 3b or 7b, or in a third composition, an effective amount of an anti-fungal agent that reduces total fungal load. In addition, the kit may comprise printed instructions to guide the user to properly use the kit.

In some embodiments, in the methods described above the level of the one or more fungal specie set forth in Table 2, 3a, 3b, 5, 7a, 7b, or 16 is determined by quantitative polymerase chain reaction (PCR).

In a third aspect, the present invention provides a method for identifying a suitable donor for FMT. The method comprises the step of determining level of one or more viral specie set forth in Table 8b in a stool sample obtained from a candidate. In some embodiments, the level of the one or more viral species is a percentage relative abundance. In some embodiments, the level of the one or more viral species set forth in Table 8b is greater than 0.1% and the candidate is identified as a suitable donor for FMT. In some embodiments, the level of the one or more viral species set forth in Table 8a is no greater than 0.1%. In some embodiments, the method further comprises a step of obtaining stool material from the candidate for use in FMT. In some embodiments, the method further comprises a step of obtaining stool material from the candidate for use in FMT. In some embodiments, the level of the one or more viral species set forth in Table 8a is greater than 0.1% and the candidate is identified as an unsuitable donor for FMT. In some embodiments, the method further comprises a step of determining total viral load in the stool sample.

In some embodiments, the level of the one or more viral specie set forth in Table 8a or 8b is determined in a first stool sample obtained from a first candidate and in a second stool sample obtained from a second candidate. In some embodiments, the first candidate has a higher level of the one or more viral species set forth in Table 8b than the second candidate and is deemed to be a more suitable FMT donor than the second candidate. In some embodiments, the first candidate has a lower level of the one or more viral species set forth in Table 8a than the second candidate and is deemed to be a more suitable FMT donor that the second candidate.

In one related aspect, a method is provided for improving FMT efficacy. The method comprises a step of introducing an effective amount of the one or more viral species set forth in Table 8b into a composition intended for use in transplantation prior to FMT. In some embodiments, after the introducing step the level of the one or more viral species set forth in Table 8b is greater than 0.1% of total viruses in the composition. In some embodiments, the method further comprises a step of performing FMT using the composition. In some embodiments, the method further comprises the step of introducing into the composition an effective amount of an anti-fungal agent that suppresses growth of one or more fungal species set forth in Table 8a. In some embodiments, after the introducing step the level of the one or more fungal species set forth in Table 8a is less than 0.1% of total viruses in the composition. In some embodiments, the method further comprises a step of performing FMT using the composition.

In another related aspect, a method is provided for improving FMT efficacy, the method comprising administering to an FMT recipient prior to FMT an effective amount of an anti-viral agent that suppresses growth of one or more viral species set forth in Table 8a. In some embodiments, the level of the one or more viral species set forth in Table 8a is determined in a stool sample from the FMT recipient prior to administration of the anti-viral agent. In some embodiments, the level of the one or more viral species set forth in Table 8a is determined in a stool sample from the FMT recipient after administration of the anti-viral agent. In some embodiments, the method further comprises a step of administering to the recipient prior to FMT an effective amount of an agent that reduces total viral load in a stool sample taken from the recipient prior to FMT.

In yet another related aspect, a kit is provided which comprises (1) a first composition comprising donor stool; and (2) a second composition comprising (i) an effective amount of one or more viral species set forth in Table 8b; or (ii) an effective amount of an anti-viral agent that suppresses growth of one or more viral species set forth in Table 8a. In some embodiments, the first composition comprises donor stool that has been dried, frozen, and placed in a capsule for oral ingestion. In some embodiments, the kit further comprises, in the second composition an effective amount of an anti-viral agent that suppresses growth of one or more viral species set forth in Table 8a, or in a third composition an effective amount of an anti-viral agent that reduces total viral load. In addition, the kit may comprise printed instructions to guide the user to properly use the kit.

In some embodiments, in the methods described above the level of the one or more viral specie set forth in Table 8a or 8b is determined by quantitative polymerase chain reaction (PCR).

Related compositions useful in FMT with improved efficacy may comprise (1) a donor stool material containing live fecal microorganisms and (2) an anti-bacterial or fungal or viral agent that specifically suppresses the growth or proliferation of one or more of the bacterial/fungal/viral species set forth in Table 1b, 3b, 6b, 7b, 8a, or 10a but exhibits no such suppressive or inhibitory effect against other bacterial, fungal, or viral species. Instead of a broad-spectrum anti-bacterial, fungal, or viral agent, such specific anti-bacterial, fungal, viral agent may be short polynucleotide in nature (e.g., a small inhibitory RNA, microRNA, miniRNA, lncRNA, or an antisense oligonucleotide) that is capable of disrupting the expression of a key gene in the life cycle of one or more of the bacterial, fungal, or viral species shown in Table 1b, 3b, 6b, 7b, 8a, or 10a that is capable of specifically targeting the species only but not other closely related fungal species.

In addition, various methods are provided for therapeutic applications: for example, a method is provided for weight reduction in a subject, including the step of introducing into the subject's gastrointestinal tract an effective amount of (1) one or more bacterial species set forth in Table 1a, or (2) one or more fungal species set forth in Table 2 or 3a. On the other hand, a method is provided for weight reduction in a subject, including the step of introducing into the subject's gastrointestinal tract an effective amount of an inhibitor suppressing (1) one or more bacterial species set forth in Table 1b, or (2) one or more fungal species set forth in Table 3b. In some cases, the introducing step is performed by way of FMT. Optionally, a step of administering to the individual an effective amount of a broad spectrum anti-bacterial or anti-fungal agent is performed before the step of introducing beneficial bacterial or fungal species, respectively, such as FMT is performed.

Also, a method is provided for suppressing a multidrug resistant bacterium in a subject, including the step of introducing into the subject's gastrointestinal tract an effective amount of (1) one or more bacterial species set forth in Table 4 or 15, or (2) one or more fungal species set forth in Table 5 or 16. In some embodiments, the bacterium is carbapenem-resistant Enterobacteriaceae (CRE). In some embodiments, the bacterium is vancomycin-resistant enterococcus (VRE). In some cases, the introducing step is performed by way of FMT. Optionally, a step of administering to the individual an effective amount of a broad spectrum anti-bacterial or anti-fungal agent is performed before the step of introducing beneficial bacterial or fungal species, respectively, such as FMT is performed.

Further, a method is provided for treating acute graft versus host disease (aGvHD) in a subject, including a step of introducing into the subject's gastrointestinal tract an effective amount of (1) one or more bacterial species set forth in Table 6a, or (2) one or more fungal species set forth in Table 7a. On the other hand, a method is provided for treating acute graft versus host disease (aGvHD) in a subject, including a step of introducing into the subject's gastrointestinal tract an effective amount of an inhibitor suppressing (1) one or more bacterial species set forth in Table 6b, or (2) one or more fungal species set forth in Table 7b. In some cases, the introducing step is performed by way of FMT. Optionally, a step of administering to the individual an effective amount of a broad spectrum anti-bacterial or anti-fungal agent is performed before the step of introducing beneficial bacterial or fungal species, respectively, such as by FMT, is performed.

Additionally, a method is provided for treating ulcerative colitis, including a step of introducing into the subject's gastrointestinal tract an effective amount of (1) one or more bacterial species set forth in Table 10b, or (2) one or more viral species set forth in Table 8b. On the other hand, a method is provided for treating ulcerative colitis, including a step of introducing into the subject's gastrointestinal tract an effective amount of an inhibitor suppressing (1) one or more bacterial species set forth in Table 10a, or (2) one or more viral species set forth in Table 8a. In some cases, the introducing step is performed by way of FMT. Optionally, a step of administering to the individual an effective amount of a broad spectrum anti-bacterial or anti-viral agent is performed before the step of introducing beneficial bacterial or viral species, respectively, such as by FMT, is performed.

Moreover, a method is provided for treating Crohn's disease in a subject, including the step of introducing into the subject's gastrointestinal tract an effective amount of one or more fungal species set forth in Table 11, for example, by way of FMT. Optionally, a step of administering to the individual an effective amount of a broad spectrum anti-fungal agent is performed before the introducing step such as by FMT is performed.

Lastly, a method is provided for weight reduction in a subject, comprising introducing into the subject's gastrointestinal tract an effective amount of any one, two, or three of Sutterella wadsworthensis, Roseburia intestinalis, or Mitsuokella multacida, for example, the subject may receive all three, i.e., an effective amount of each of Sutterella wadsworthensis, Roseburia intestinalis, and Mitsuokella multacida. Also provided is a method for reducing total cholesterol and low-density lipoprotein cholesterol in a subject, comprising introducing into the subject's gastrointestinal tract an effective amount of any one, two, or three of Sutterella wadsworthensis, Roseburia intestinalis, or Mitsuokella multacida, for example, the subject may receive all three, i.e., an effective amount of each of Sutterella wadsworthensis, Roseburia intestinalis, and Mitsuokella multacida. This method of cholesterol reduction may be practiced independent of weight loss or concurrent with weight loss in the subject. These methods may be performed by way of FMT. Optionally, a step of administering to the individual an effective amount of a broad spectrum anti-bacterial agent such as antibiotic is performed before the introducing step such as FMT is performed. For use in this method, a composition comprising an effective amount of any one, two, or three of Sutterella wadsworthensis, Roseburia intestinalis, or Mitsuokella multacida is provided, e.g., such a composition may be derived from donor stool material and has been dried, frozen, and placed in a capsule for oral ingestion.

### BRIEF DESCRIPTION OF THE DRAWINGS

FIG. 1a “Favourable” bacteria present in donors for FMT use to induce weight loss identified by correlation (Linear Regression). Five species were identified to be positively correlated with weight loss by linear regression. These species are presented in Table 1a. FIG. 1b “Unfavourable” bacteria present in donors for FMT use to induce weight loss identified by correlation (Linear Regression). Seventeen species were identified from linear regression to be negatively correlated with weight loss. The species are presented in Table 1b. FIGS. 1c and 1d “Favourable” and “Unfavourable” bacteria present in donors for FMT use to induce weight loss identified by LEfSe analysis. FIG. 1c LefSe plot showing bacteria that were significantly different in abundance in “favourable” and “less favourable” donors using median as cutoff point. FIG. 1d LefSe plot showing bacteria that were significantly different in abundance in “favourable” and “less favourable” donors using mean as cutoff point. The species are presented in Table 1a and Table 1b.

FIG. 2 “Favourable” fungi in donors for the use of FMT to induce weight loss. LEfSe plot showing that relative abundance of Cercospora beticola and Kazachstania naganishii were significantly higher in controls than in obese subjects. These species are presented in table 2.

FIG. 3 Fungal species in recipient associated with successful and unsuccessful weight loss. LEfSe plot showed that relative abundance of fungal species that were significantly higher in “weight loss” or “non-weight-loss” group. These species are presented in table 3a and 3b.

FIG. 4a “Favourable” bacteria in donors for the use of FMT to treat CRE identified in cross-sectional study. Taxonomic cladogram from LEfSe analysis of metagenomics sequencing comparing of bacteria profile in patients with CRE and healthy control. Comparison of bacteria profile with linear discriminant analysis effect size (LEfSe) model. Only the taxa meeting a linear discriminant analysis (LDA) threshold value of >2 was considered significant. Relative abundance of 33 species were significantly higher in healthy control (green). These species are presented in Table 4. FIG. 4b “Favourable” bacteria in donors for the use of FMT to treat CRE identified in prospective pilot study. Comparing taxonomic profile of bacteria species in FMT donor and pre- and post-FMT of recipients based on metagenomics sequencing. Relative abundance of bacterial taxa present in fecal samples of donor and pre- and post-FMT of recipients. R1 and R2 referred to stool samples collected from FMT recipient 1 and 2 respectively. Columns are labelled by the letter D followed by the sample collection day, where first FMT was performed on day 0. These species are presented in Table 4.

FIG. 5 “Favourable” fungi in donors for the use of FMT to treat CRE identified in prospective pilot trial. Comparison of taxonomic profile of fungal species in FMT donor and pre- and post-FMT of recipients with CRE based on metagenomics sequencing. Relative abundance of fungal taxa present in fecal samples of donor and pre- and post-FMT of recipients. R1 and R2 referred to stool samples collected from FMT recipient 1 and 2 respectively. Columns are labelled by the letter D followed by the sample collection day, where first FMT was performed on day 0. Detail relative abundance of selected species was shown in Table 5.

FIG. 6a “Favourable” and “Unfavourable” bacteria in donors for the use of FMT to treat acute Graft versus host disease. Comparison of taxonomic profile of bacterial species in FMT donor, and pre- and post-FMT in recipient with aGvHD based on metagenomics sequencing. Bar chart represents relative abundance of bacterial taxa in fecal samples from donor, and pre- and post-FMT of recipients. Fecal samples collected from recipient were labelled by the sample collection day, where 0 represent the day of first FMT. Fecal samples from donor that were used in FMT were labelled as D4 and D8. There were 5 samples from donor D8 collected on different days. Corynebacterium jeikeium (71.2%) was dominated in the recipient's gut bacterial community pre-FMT, and decreased substantially to <0.1% after the first FMT. Engraftment of 7 bacterial species was observed in the patient after receiving repeated FMT and these species took up a large proportion of the patient's bacterial community. These species were Alistipes onderdonkii, Alistipes putredinis, Clostridium bolteae, Clostridium nexile, Clostridium symbiosum, Eggerthella unclassified, Ruminococcus gnavu. These species are listed in Table 6a and Table 6b. FIG. 6b “Favourable” and “Unfavourable” bacteria in donors for the use of FMT to treat acute Graft versus host disease identified by LEfSe analysis. LEfSe plot showed that relative abundance of 4 bacterial species differed significantly when the patient had diarrhea or no diarrhea. A total of 22 stool samples were collected pre- and post-FMT from the patient with aGvHD. Samples were classified according to whether the patient had or did not had diarrhea on the day of stool collection. Diarrhea was defined as having 2 or more bowel opening per day, while non-diarrhea was defined as having 0 or 1 bowel opening per day. LEfSe analysis was used to identify bacterial species that were significantly different between stool samples from “diarrhea” or “non-diarrhea” group. Only the taxa meeting a LDA threshold value of >2 was considered significant. Eubacterium rectale was significantly higher in diarrhea group. Alistipes putredinis, Alistipes onderdonkii and Clostridium hathewayi were significantly higher in non-diarrhea group. These species are listed in Table 6a and Table 6b.

FIG. 7 “Favourable” and “Unfavourable” fungal species in donors for using FMT to treat aGvHD identified from heatmap. Comparison of taxonomic profile of fungal species in FMT donor and in recipient with aGvHD pre and post FMT based on metagenomics sequencing. Relative abundance of fungal taxa present in fecal samples of donor and recipient with aGVHD pre and post FMT. Fecal samples collected from recipient were labelled by the sample collection day, where 0 represent the day of first FMT. Fecal samples from donor that were used in FMT were labelled as D4 and D8. There were 5 samples from donor D8 collected on different days. Engraftment of fungi species from donor in the patient with aGvHD after FMT was assessed by heat map. 44 fungi species were engrafted after FMT treatment in the patient. Blue to red colour shades indicate increasing relative abundance as indicated in legend. Taxonomic labels on right of figure indicate labels of indicated species. Representative species are listed in Table 7a and 7b.

FIG. 8 Differential viral taxa between mucosa of healthy population and patients with UC at the family, genus and species levels. Differentially enriched viral families (a), genera (b) and species (c) between health and UC mucosa were determined by DESeq analysis with FDR correction (only those differential taxa with adjusted p value <0.05 and |Log2 (between-group fold-change) |>2 are shown). For viral taxon names, taxa color-coated by black denote prokaryotic viruses, while those color-coated by orange denote eukaryotic viruses. For viral abundance box plots, the boxes extend from the 1st to 3rd quartile (25th to 75th percentile), with the median depicted by a vertical line. These taxa are listed in Table 8a and 8b.

FIG. 9 Mucosal virome enterotypes in healthy population and patients with UC. FIG. 9a Virome enterotype clustering based on partition around medoids (PAM) algorithm and principal coordinates analysis (PCoA) on the viral community structures of mucosa from healthy population and patients with UC. The inset shows the ratio of healthy individuals and UC subjects within each enterotype population. FIG. 9b Heatmap of the presence of differential viral species contributing to clustering of the 2 mucosal virome enterotypes. Discriminative species were identified by concordant DEseq and Random Forest analyses. Viral species abundances are color intensified according to Logio RPKM values. Only those species concordantly determined by DEseq and Random Forest algorithm with effect size more than 2 and FDR-adjusted P value <0.05 are shown. These species are listed in Table 9a and 9b.

FIG. 10 Altered bacterial microbiota in UC mucosa. FIG. 10a Comparison of bacteria a diversities based on Simpson diversity and Chaol richness in the mucosa of controls and UC subjects. Statistical significance was determined by t test, *P<0.05, **P<0.01. For box plots, the boxes extend from the 1st to 3rd quartile (25th to 75th percentile), with the median depicted by a vertical line. The bacteria composition in health and UC mucosa was plotted in relative abundance, at the phylum (b), family (c) and genus (d) levels. e, Differentially enriched bacterial taxa between the mucosa of health individuals and UC subjects were determined by Lefse analysis with FDR correction. Only those taxa with adjusted P value <0.05 and LDA effect size >2 are shown. These taxa are listed in Table 10a and 10b.

FIG. 11 Study timeline of mouse model. Mice were fed with high-fat diet for 1 month, followed by normal diet for 1 month and 7 days of antibiotics treatment. Mice were then divided into 3 groups and received treatment of Sutterella wadsworthensis, Roseburia intestinalis, Mitsuokella multacida, Consortium (at dose of dose of 1*10⁹ cfu/ml), or culture medium via oral gavage.

FIG. 12 Administration of Sutterella wadsworthensis significantly reduced body weight gain while and administration of Roseburia intestinalis showed a trend of reduced body weight gain. Effect is particular obviously after day 15 (after 3 oral gavage). Abbreviation: SW-Sutterella wadsworthensis; RI-Roseburia intestinalis

FIG. 13 Both Sutterella wadsworthensis and Roseburia intestinalis reduced body weight gain at day 20 and day 45. Reduction of body weight gain continues 20 days after cessation of treatment (at day 45). Body weight at day 20 and day 45 were compared with day 0, and presented as percentage of body weight gain. Abbreviation: SW-Sutterella wadsworthensis; RI-Roseburia intestinalis

FIG. 14 Average food intake showed a decreasing trend after administration of consortium, MM, RI and SW, in contrast to administration of medium only which showed an increasing trend. The effect of reduction of food intake is most obvious in RI and SW. Average food intake was calculated as gram of food intake per mouse per day.

FIG. 15 Average food efficiency were reduced after day 15 in mice administered with consortium, MM, RI and SW compared to administration of medium. Average food efficiency was calculated as the body weight gain (gram) in 5 days per total food intake (gram) in these 5 days per mouse.

FIG. 16 Administration of Sutterella wadsworthensis alone reduced LDL-C, TC and TG. Administration of Roseburia intestinalis alone reduced LDL-C and TC.

Administration of a consortium of Sutterella wadsworthensis, Roseburia intestinalis and Mitsuokella multacida reduced LDL-C and TC. All results are compared to administration of medium-control on day 45. Abbreviation: SW-Sutterella wadsworthensis; RI-Roseburia intestinalis; MM: Mitsuokella multacida; LDL-C: low-density lipoprotein cholesterol; TC: total cholesterol; TG: total triglyceride.

FIG. 17 Timeline of sample collection for donor and recipients. Relative sample collective time and results for CRE for each recipient are indicated.

FIG. 18 Relative abundance alterations of bacterial species, Ruminococcus sp_5_1_39BFAA, Collinsella tanakaei and Eubacterium sicaeum, in three FMT recipients before and after FMT and in their corresponding donors. Relative abundance of Ruminococcus sp_5_1_39BFAA, Collinsella tanakaei and Eubacterium sicaeum were undetectable before FMT and increased after FMT to a level similar to donor or even higher.

FIG. 19 LEfSe analysis comparing bacterial composition in pre-FMT (green) and post-FMT (red). Represented are all taxa significantly distinct with LDA scores >2.0.

FIG. 20 Analysis of fungal composition of donors and recipients. (a) Alpha diversity (Shannon's diversity) for donors, recipients, and recipient post-FMT at different times. (b) Principal Coordinate Analysis (PCoA) based on Bray-Curtis distance for recipients. Plot showed the distance between donor and CRE recipients over time. (c) LEfSe analysis comparing mycobiome composition in pre-FMT (yellow) and post-FMT (blue). (d) Heatmap showing the dominant species significantly presented in pre- and post-FMT fecal samples.

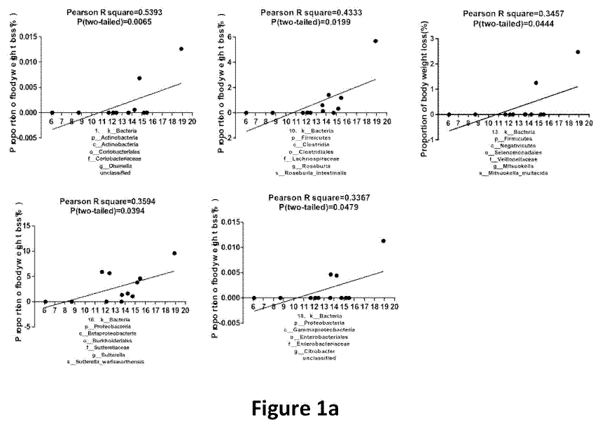

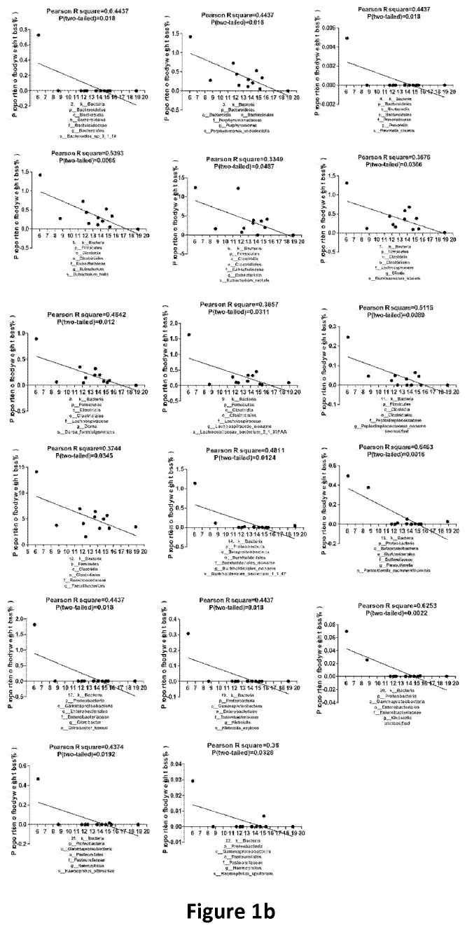

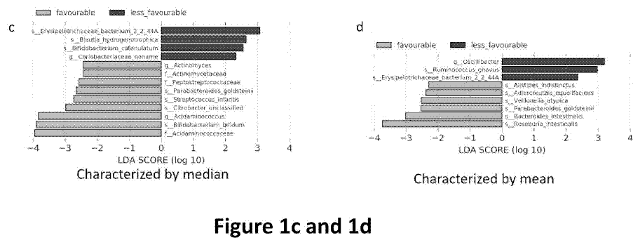

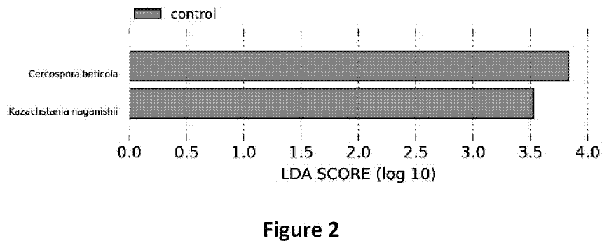

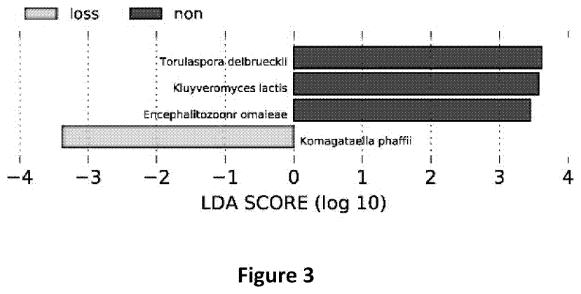

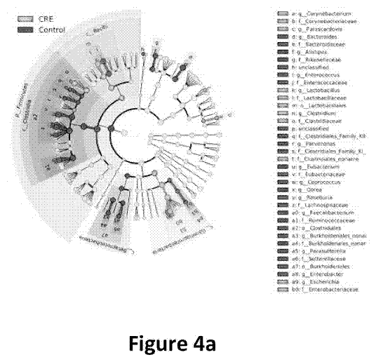

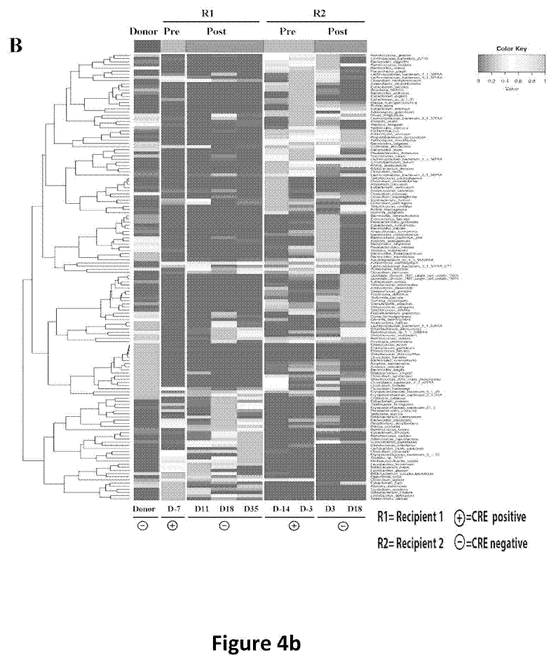

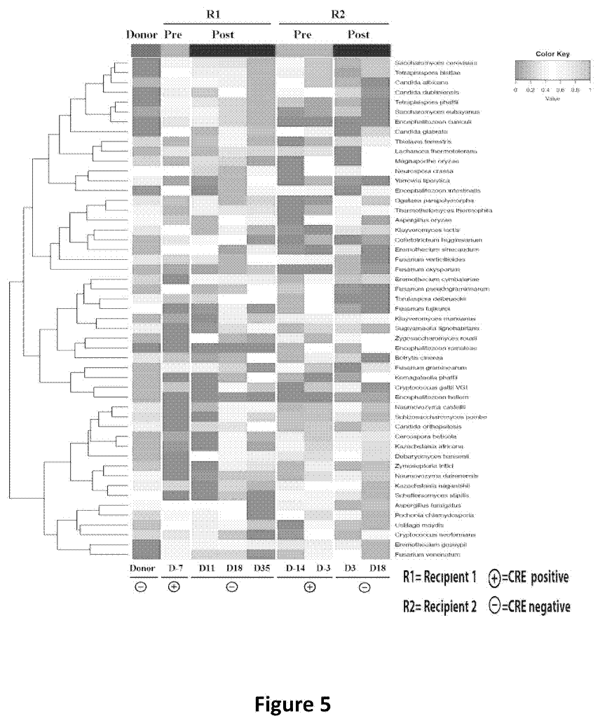

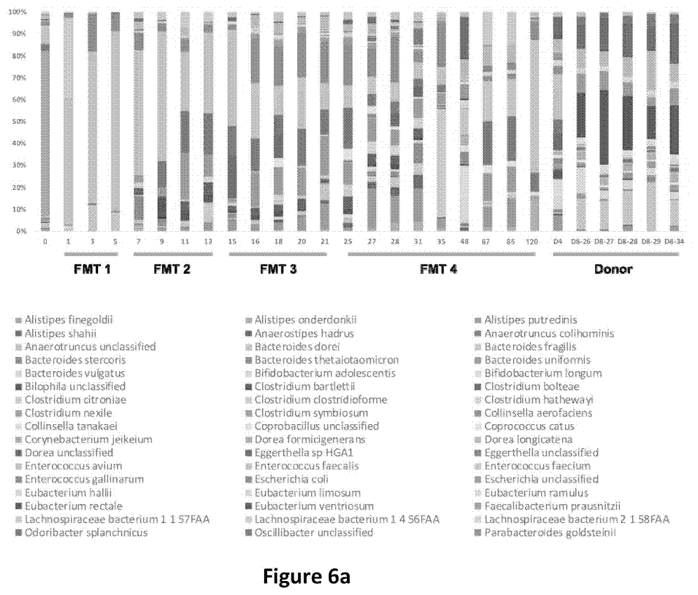

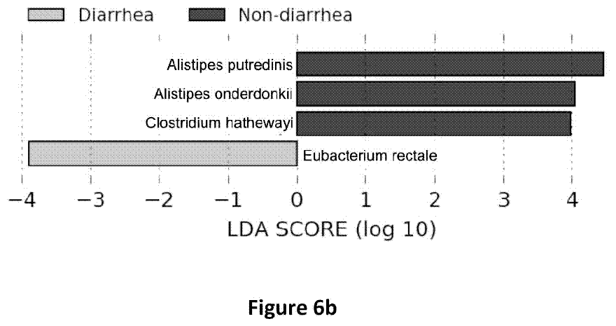

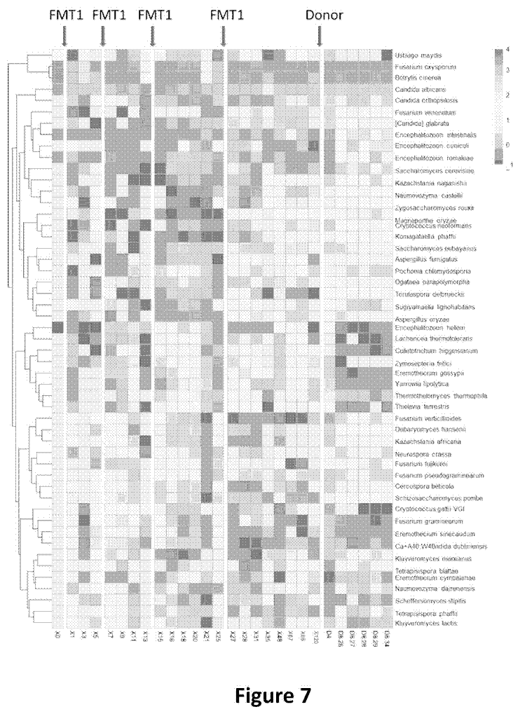

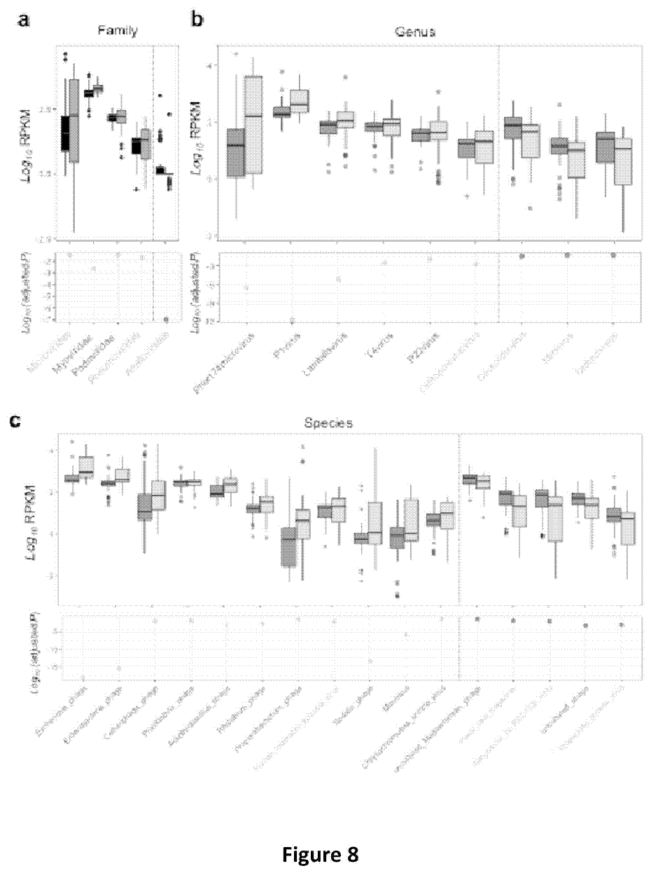

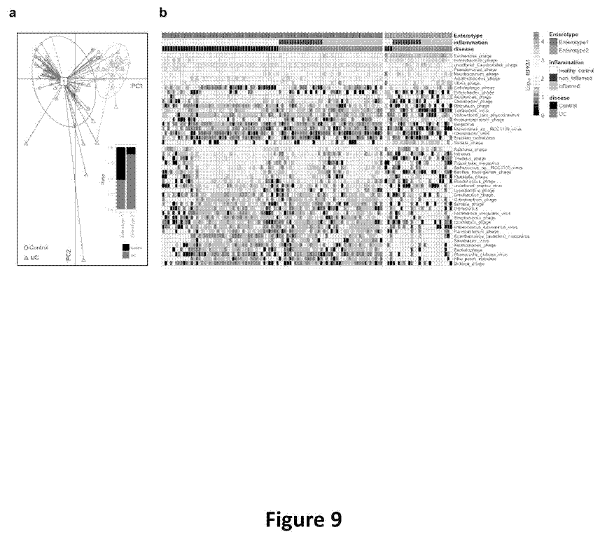

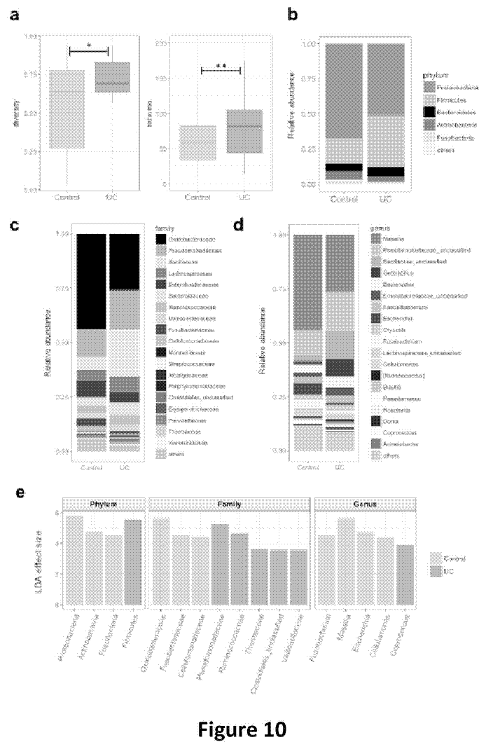

### Definitions

The term “fecal microbiota transplantation (FMT)” or “stool transplant” refers to a medical procedure during which fecal matter containing live fecal microorganisms (bacteria, fungi, viruses, and the like) obtained from a healthy individual is transferred into the gastrointestinal tract of a recipient to restore healthy gut microflora that has been disrupted or destroyed by any one of a variety of medical conditions. Typically, the fecal matter from a healthy donor is first processed into an appropriate form for the transplantation, which can be made through direct deposit into the lower gastrointestinal tract such as by colonoscopy, or by nasal intubation, or through oral ingestion of an encapsulated material containing processed (e.g., dried and frozen) fecal matter. FMT is used for treating a number of medical conditions including obesity, metabolic syndrome, gastrointestinal disorders (such as inflammatory bowel disease (IBD) including ulcerative colitis (UC) and Crohn's disease (CD)), antibiotic-resistant bacterial infections (such as Clostridium difficile infection (CDI) or conditions caused by multidrug-resistant organisms including carbapenem-resistant Enterobacteriaceae (CRE) or vancomycin-resistant Enterococcus (VRE)), as well as autism, depression, obesity, diabetes, alopecia, acute graft-versus-host disease (aGvHD), and further including certain neurological conditions such as multiple sclerosis and Parkinson's Disease.

The term “inhibiting” or “inhibition,” as used herein, refers to any detectable negative effect on a target biological process, such as RNA/protein expression of a target gene, the biological activity of a target protein, cellular signal transduction, cell proliferation, and the like. Typically, an inhibition is reflected in a decrease of at least 10%, 20%, 30%, 40%, 50%, 60%, 70%, 80%, 90% or greater in the target process (e.g., growth or proliferation of a microorganism of certain species, for example, one or more of the bacterial species shown in Table 1b, 6b, or 10a; or fungal species shown in Table 3b or 7b; or viral species shown in Table 8a), or any one of the downstream parameters mentioned above, when compared to a control. “Inhibition” further includes a 100% reduction, i.e., a complete elimination, prevention, or abolition of a target biological process or signal. The other relative terms such as “suppressing,” “suppression,” “reducing,” and “reduction” are used in a similar fashion in this disclosure to refer to decreases to different levels (e.g., at least 10%, 20%, 30%, 40%, 50%, 60%, 70%, 80%, 90% or greater decrease compared to a control level) up to complete elimination of a target biological process or signal. On the other hand, terms such as “activate,” “activating,” “activation,” “increase,” “increasing,” “promote,” “promoting,” “enhance,” “enhancing,” or “enhancement” are used in this disclosure to encompass positive changes at different levels (e.g., at least 10%, 20%, 30%, 40%, 50%, 60%, 70%, 80%, 90%, 100%, 200%, or greater such as 3, 5, 8, 10, 20-fold increase compared to a control level, for example, the control level of one or more of the bacterial species shown in Table 1a, 4, 6a, 10b, 11, or 15; or fungal species shown in Table 2, 3a, 5, 7a, or 16; or viral species shown in Table 8b) in a target process or signal.

The term “anti-bacterial/fungal/viral agent” refers to any substance that is capable of inhibiting, suppressing, or preventing the growth or proliferation of bacterial, fungal, or viral species, respectively, especially those of shown in Tables 1b, 3b, 6b, 7b, 8a, and 10a. Known agents with anti-bacterial activity include various antibiotics that generally suppress the proliferation of a broad spectrum of bacterial species as well as agents such as antisense oligonucleotides, small inhibitory RNAs, and the like that can inhibit the proliferation of specific bacterial species. The term “anti-bacterial/fungal/viral agent” is similarly defined to encompass both agents with broad spectrum activity of killing virtually all species of bacteria, fungi, or viruses, respectively, and agents that specifically suppress proliferation of target bacteria, fungal, or viral species, respectively.

“Percentage relative abundance,” when used in the context of describing the presence of a particular bacterial or fungal or viral species (e.g., any one of those shown in any one of Tables 1-11) in relation to all bacterial or fungal or viral species, respectively, present in the same environment, refers to the relative amount of the bacterial or fungal or viral species out of the amount of all bacterial or fungal or viral species, respectively, as expressed in a percentage form. For instance, the percentage relative abundance of one particular fungal species can be determined by comparing the quantity of DNA specific for this species (e.g., determined by quantitative polymerase chain reaction) in one given sample with the quantity of all fungal DNA (e.g., determined by quantitative polymerase chain reaction (PCR) and sequencing based on the Internal transcribed spacer 2 or ITS2 sequence) in the same sample.

“Absolute abundance,” when used in the context of describing the presence of a particular bacterial or fungal or viral species (e.g., any one of those shown in Tables 1-11, 15, and 16) in the feces, refers to the amount of DNA derived from the bacterial or fungal or viral species, respectively, out of the amount of all DNA in a fecal sample. For instance, the absolute abundance of one fungus can be determined by comparing the quantity of DNA specific for this fungal species (e.g., determined by quantitative PCR) in one given sample with the quantity of all fecal DNA in the same sample.

“Total bacterial/fungal/viral load” of a fecal sample, as used herein, refers to the amount of all bacterial/fungal/viral DNA, respectively, out of the amount of all DNA in the fecal sample. For instance, the absolute abundance of fungi can be determined by comparing the quantity of fungal specific DNA (e.g., 18S rDNA determined by quantitative PCR) in one given sample with the quantity of all fecal DNA in the same sample.

The term “effective amount,” as used herein, refers to an amount of a substance that produces a desired effect (e.g., an inhibitory or suppressive effect on the growth or proliferation of one or more detrimental bacterial or fungal or viral species (e.g., the bacterial species shown in Table 1b, 6b, or 10a) for which the substance (e.g., an anti-bacterial agent) is used or administered. The effects include the prevention, inhibition, or delaying of any pertinent biological process during bacterial/fungal/viral proliferation to any detectable extent. The exact amount will depend on the nature of the substance (the active agent), the manner of use/administration, and the purpose of the application, and will be ascertainable by one skilled in the art using known techniques as well as those described herein. In another context, when an “effective amount” of one or more beneficial bacterial or fungal or viral species (e.g., those listed in Table 1a, 2, 3a, 4, 5, 6a, 7a, 8b, 10b, 11, 15, or 16) are artificially introduced into a composition intended for use in FMT, it is meant that the amount of the pertinent bacteria/fungi/viruses being introduced is sufficient to confer to the FMT recipient health benefits such as weight loss, improved sensitivity to insulin, reduced blood cholesterol level, suppression of drug-resistant bacterial infection, and/or alleviation of acute graft-versus-host disease (aGvHD) and GI tract diseases such as IBD.

As used herein, the term “about” denotes a range of value that is +/−10% of a specified value. For instance, “about 10” denotes the value range of 9 to 11 (10+/−1).

### DETAILED DESCRIPTION OF THE INVENTION

### I. Introduction

The invention provides a novel approach for assessing the likelihood of effective FMT prior to the procedure being performed as well as for improving the effectiveness of the FMT procedure in conferring certain health benefits to the recipients. During their studies, the present inventors discovered that the presence and relative abundance of certain bacterial, fungal, and viral species both in a recipient's gastrointestinal tract and in a donor's stool directly correlate with the outcome of FMT. For example, the presence of bacterial species shown in Table 1a, 4, 6a, 10b, 11, or 15, especially at an elevated level, are found to confer health benefits to the FMT recipients, whereas the presence of bacterial species shown in Table 1b, 6b, or 10a, especially at an elevated level, tends to negatively impact the FMT outcome. Similarly, the presence and relative abundance of certain fungal and viral species correlated with desirable FMT outcome have been identified. The detection of one or more “beneficial” species of bacteria/fungi/viruses such as those shown in Table 1a, 2, 3a, 4, 5, 6a, 7a, 8b, 10b, 11, 15, or 16, especially at an elevated level; on the other hand, the detection of one or more “detrimental” species of bacteria/fungi/viruses such as those shown in Table 1b, 3b, 6b, 7b, 8a, 10a, or 16, especially at an elevated level, in a potential donor's stool thus can be used to guide donor selection, whereas analysis of the level of these pertinent species of bacteria/fungi/viruses in an FMT recipient can determine whether the subject is immediately ready for FMT or should be treated with an anti-bacterial/fungal/viral agent that suppresses such undesirable microorganism prior to FMT in order to optimize the therapeutic outcome.

### II. FMT Donor/Recipient Selection and Preparation

Patients suffering from CDI, especially recurring CDI, are often considered as recipients for FMT treatment. Aside from CDI, other diseases and conditions, including those of digestive system or nervous system such as colitis, irritable bowel syndrome (IBS), Crohn's disease, acute graft-versus-host disease (aGvHD), infections caused by multidrug-resistant bacteria such as CRE or VRE, multiple sclerosis, Parkinson's Disease, diabetes mellitus, and obesity are also suitable for FMT treatment.

Fecal matter used in FMT is obtained from a healthy donor and then processed into appropriate forms for the intended means of delivery in the upcoming FMT procedure. Up until very recently, the general criterion for an FMT donor has been simply that the donor is a healthy individual without any known diseases or disorders especially in the digestive tract, although some preference is often given to the members of the same household as the recipient.

The present inventors have discovered in their studies that elevated presence of one or more “beneficial” bacterial species, such as those shown in Table 1a, in a recipient's gastrointestinal tract or in a donor stool (which is used in the transplantation after being processed) can confer significant health benefits following FMT treatment in a patient, such as body weight loss, improved insulin sensitivity, reduced blood/serum/plasma cholesterol level in the recipient. In contrast, no such healthful benefits are conferred after FMT treatment when elevated presence of other “detrimental” bacterial species, such as those shown in Table 1b, is found in a recipient's GI tract after FMT or in a donor stool used in FMT. Similarly, elevated presence of one or more “beneficial” fungal species, such as those shown in Table 2 or 3a, in a recipient's gastrointestinal tract or in a donor stool used in FMT can confer the same or similar health benefits following FMT treatment in a patient, whereas no such health benefits is obtained when elevated presence of “detrimental” fungal species, such as those shown in Table 3b, is found in a recipient's GI tract after FMT or in a donor stool used in FMT.

This revelation enables the initial screening of individuals as appropriate FMT donors as well as the initial screening of patients as likely candidates for successful and beneficial FMT treatment, especially in the case of treatment for obesity or metabolic syndrome including insulin insensitivity and/or type II diabetes: if a candidate donor's stool contains a minimal or elevated level of any one or multiple bacterial species shown in Table 1a (e.g., each is greater than 0.1%, 0.2%, 0.4%, 0.6%, 0.8%, 1%, 1.2%, 1.5%, 1.8%, or 2% of total bacteria), the candidate is deemed as suitable as an FMT donor, and his stool can immediately retrieved for processing and later used in FMT; on the other hand, if a candidate's stool sample shows no or only low level of these beneficial bacterial species (e.g., each is no greater than 0.1%, 0.2%, 0.4%, 0.6%, 0.8%, 1%, 1.2%, 1.5%, 1.8%, or 2% of total bacteria), then the candidate is deemed not an immediately appropriate FMT donor and his fecal material should not be taken for use in FMT without necessary modification before potential use. One possible means of improving donor fecal material prior to processing for use in FMT is to artificially introduce one or more of the beneficial bacterial species (e.g., those in Table 1a) so as to increase the presence of such bacterial species in the fecal material for use in FMT (e.g., each species is greater than 0.1%, 0.2%, 0.4%, 0.6%, 0.8%, 1%, 1.2%, 1.5%, 1.8%, or 2% of total bacteria).

Conversely, the inventors have also revealed that the absence or reduced presence of certain undesirable or detrimental bacterial species, including one or more of those shown in Table 1b, in the fecal material used in FMT, tends to yield significant health benefits following FMT treatment in a patient, such as body weight loss, improved insulin sensitivity, reduced blood/serum/plasma cholesterol level in the recipient. Thus, if a candidate donor's stool sample has been tested and shown to contain a reduced or undetectable level of one or more of the bacterial species shown in Table 1b (e.g., each is no greater than 0.02%, 0.04%, 0.06%, 0.08%, 0.1%, 0.2%, 0.4%, or 0.5% of total bacteria), the individual is deemed an appropriate donor and his stool samples can be immediately collected and processed for use in FMT. On the other hand, if an individual as a potential donor has his stool tested and found to contain a significant presence, especially an elevated level, of any one or more of the bacterial species shown in Table 1b (e.g., each is greater than 0.02%, 0.04%, 0.06%, 0.08%, 0.1%, 0.2%, 0.4%, or 0.5% of total bacteria), he is deemed inappropriate as donor of fecal matter for use in FMT, thus his stool samples should not be taken for use in FMT.

Ideally, a desirable FMT composition prepared from a donor fecal material and intended for use in FMT for treating obesity or metabolic syndrome or type II diabetes has both a high level of one or more beneficial bacterial species (e.g., those shown in Table 1a) and a low level of one or more detrimental bacterial species (e.g., those shown in Table 1b). Thus, while modifications can be made to a composition intended for use in FMT to separately address the insufficient level of beneficial bacteria and the over-presence of detrimental bacteria, one possible modification is to increase the level of one or more beneficial bacterial species (e.g., those shown in Table 1a), for example, by supplementing the transfer material intended for use in FMT or by directly introducing into the recipient's GI tract an adequate amount of such beneficial bacterial species, optionally while at the same time to suppress the level of one or more detrimental bacterial species (e.g., those shown in Table 1b) in order to maximize the potential health benefits a recipient may derived from the FMT procedure.

Insofar as FMT recipients are concerned, if a patient who has been proposed to receive FMT treatment for obesity or metabolic syndrome or type II diabetes, and his stool sample shows an elevated level of one or more of the detrimental bacterial species shown in Table 1b (e.g., each is greater than 0.02%, 0.04%, 0.06%, 0.08%, 0.1%, 0.2%, 0.4%, or 0.5% of total bacteria), then the patient may be deemed unsuitable to receive FMT right away, as the therapy may have a diminished chance to yield health benefits such as reduced body weight, increased insulin sensitivity, reduced blood cholesterol. The patient may first receive treatment by an anti-bacterial agent to reduce the level of detrimental bacterial species, especially those shown in Table 1b, in his gastrointestinal tract, prior to the start of FMT treatment, during which the patient is to receive donor fecal material enriched with one or more of the beneficial bacterial species (such as those shown in Table 1a) and/or with suppressed level of one or more detrimental bacterial species (e.g., those shown in Table 1b). On the other hand, if the patient who is proposed to receive FMT treatment does not have a significant presence, especially elevated presence, of any one or more of the bacterial species shown in Table 1b (e.g., each is no greater than 0.02%, 0.04%, 0.06%, 0.08%, 0.1%, 0.2%, 0.4%, or 0.5% of total bacteria), then the patient may proceed to begin FMT treatment right away without other steps of preparation or pre-treatment.

In addition, the inventors have also identified “beneficial” and “detrimental” fungal species for the FMT treatment for obesity or metabolic syndrome or type II diabetes, which can guide the selection of appropriate FMT donors and recipients in the same fashion as described above: for example, a desirable FMT composition prepared from a donor fecal material and intended for use in FMT for treating obesity or metabolic syndrome or type II diabetes has an elevated level of one or more beneficial fungal species (e.g., those shown in Table 2 or 3a, each no less than 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 11%, 12%, 13%, 14%, or 15% of total fungi) and/or a low level of one or more detrimental bacterial species (e.g., those shown in Table 3b, each no greater than 0.05%, 0.1%, 0.2%, 0.3%, 0.4%, 0.5%, 0.6%, 0.7%, 0.8%, 0.9%, 1%, 1.5%, 2%, 2.5%, 3%, or 3.5% of total fungi). Further, preparation either for FMT donor/donor fecal material or for recipient can be carried out to manipulate the level of beneficial fungal species (cause an increase) and/or the level of detrimental fungal species (cause a decrease) in order to optimize the health benefits from the FMT procedure, similar to the above description of the manipulation of beneficial and/or detrimental bacterial species prior to FMT.

In the case of FMT treatment for suppressing or eradicating multidrug resistant bacteria, such as carbapenem-resistant Enterobacteriaceae (CRE) and vancomycin-resistant enterococcus (VRE), the inventors have identified “beneficial” bacterial and fungal species, which, when present in an elevated level in FMT donor stool, allow the procedure to achieve enhanced therapeutic efficacy. These bacterial and fungal species are set forth in Tables 4 and 5, respectively. A desirable FMT composition prepared from a donor fecal material and intended for use in FMT for treating multi-drug resistant bacteria has an elevated level of one or more beneficial bacterial or fungal species (e.g., those shown in Table 4, 5, 15, or 16, each no less than 0.02%, 0.05%, 0.1%, 0.5%, 1%, 1.5%, 2%, 2.5%, or 3% of total bacteria or total fungi). Further, preparation either for FMT donor/donor fecal material or for recipient can be carried out to manipulate the level of beneficial bacterial or fungal species (cause an increase) in order to maximize the efficacy and health benefits from the FMT procedure, similar to the above description of the manipulation of beneficial bacterial species prior to FMT, for example, by artificially introducing an added and effective amount of one or more of the “beneficial” bacterial and/or fungal species of Table 4, 5, 15, or 16 into the transfer material to be used in FMT or directly to the recipient's GI tract.

In the case of FMT treatment for cell or tissue transplant-related conditions such as acute graft-versus-host disease (aGvHD), the inventors have identified certain “beneficial” bacterial and fungal species, which, when present at an elevated level in a recipient's stool after FMT treatment, correlate with enhanced therapeutic efficacy. These bacterial and fungal species are set forth in Tables 6a and 7a, respectively. On the other hand, certain “detrimental” bacterial and fungal species are also identified, see Table 6b and 7b. Their presence at an elevated level in a recipient's stool after FMT treatment correlates with diminished or inadequate therapeutic outcome. Thus, when selecting potential FMT donors, one whose fecal sample contains an elevated level of any one or more of the “beneficial” bacterial and/or fungal species (set forth in Table 6a or 7a) over an average (e.g., greater than 0.1%, 0.2%, 0.5%, 0.8%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 11%, 12%, 13%, 14%, or 15% of total bacteria or total fungi) or the corresponding level(s) found in another potential donor's fecal sample will be deemed more suitable donors. Conversely, a suitable donor should also have in his stool sample a lower level of one or more or all of the “detrimental” bacterial and/or fungal species set forth in Table 6b or 7b. Further, preparation either for FMT donor/donor fecal material or for recipient can be carried out to manipulate the level of beneficial bacterial or fungal species (cause an increase) and/or the level of detrimental bacterial or fungal species (cause a decrease) in order to maximize the efficacy and health benefits from the FMT procedure, similar to the above description of the manipulation of beneficial bacterial species prior to FMT, for example, by artificially introducing an added and effective amount of one or more of the “beneficial” bacterial and/or fungal species of Table 6a or 7a into the transfer material to be used in FMT or directly to the recipient's GI tract. Means for suppression, especially specific suppression, of one or more or all of the detrimental bacterial and/or fungal species set forth in Table 6b or 7b in recipients' GI tract following FMT may be applied to enhance therapeutic efficacy or benefits.

In the case of GI tract disorders or conditions such as inflammatory bowel disease (IBD), specifically ulcerative colitis (UC), the inventors have identified certain bacterial and viral species the presence of which, especially at an elevated level, are correlated with the presence of disease. These bacterial and viral species are set forth in Tables 8a and 10a, respectively. On the other hand, certain other bacterial and viral species are also identified, see Table 8b and 10b, respectively, the absence or presence at a reduced level of which have been observed in an individual suffering from the disease in comparison with a healthy counterpart. Thus, these particular bacterial and viral species may be used to guide selection of FMT donors for the purpose of providing material for use in treating GI disorders such as IBD, which includes both UC and CD: a lower level of one or more of the bacterial species and/or viral species set forth in Table 8a or 10a tends to indicate suitability of a candidate as an FMT donor. Conversely, a higher level of one or more of bacterial species and/or viral species set forth in Table 8b or 10b tends to indicate suitability of a candidate as an FMT donor. Further, one potential donor whose level in his stool sample of one or more of the bacterial species and/or viral species set forth in Table 8a or 10a is lower than the corresponding level found in another candidate's stool sample, he will be deemed a more appropriate donor than the second candidate. On the other hand, when one potential donor whose level in his stool sample of one or more of the bacterial species and/or viral species set forth in Table 8b or 10b is higher than the corresponding level found in another candidate's stool sample, he will be deemed a more appropriate donor than the second candidate. Further manipulation of these relevant bacterial and/or viral species in the FMT material and/or in a recipient's GI tract, for example, to introduce an additional and effective amount of one or more of the bacterial and viral species are set forth in Tables 8b and 10b, or to reduce in the FMT material and/or in a recipient's GI tract the amount of the bacterial and viral species are set forth in Tables 8a and 10a, may be used for enhancing the therapeutic effects of FMT therapy strategies for the treatment of IBD especially UC.

In the case of FMT treatment of GI tract disorders or conditions such as Crohn's Disease (CD), which is another type of inflammatory bowel disease (IBD), the inventors have identified certain “beneficial” bacterial species, which, when present in an elevated level in FMT donor stool, allow the procedure to achieve enhanced therapeutic efficacy. These bacterial species are set forth in Table 11. A desirable FMT composition prepared from a donor fecal material and intended for use in FMT for treating CD has an elevated level of one or more of the beneficial bacterial species (e.g., those shown in Table 11, each reaching no less than 0.1%, 0.2%, or 0.5% of total bacteria). Further, preparation either for a FMT donor/donor fecal material or for a recipient can be carried out to manipulate the level of one or more of the beneficial bacterial species (cause an increase) in order to maximize the efficacy and health benefits from the FMT procedure, similar to the above description of the manipulation of beneficial bacterial species prior to FMT, for example, by artificially introducing an added and effective amount of one or more of the “beneficial” bacterial species of Table 11 into the transfer material to be used in FMT or directly to the recipient's GI tract.

Various methods have been reported in the literature for determining the levels of all bacterial or fungal or viral species in a sample, for example, amplification (e.g., by PCR) and sequencing of bacterial polynucleotide sequence taking advantage of the sequence similarity in the commonly shared 16S rDNA bacterial sequences. On the other hand, the level of any given bacterial species may be determined by amplification and sequencing of its unique genomic sequence. A percentage abundance is often used as a parameter to indicate the relative level of a bacterial species in a given environment.

### III. Treatment Methods Using Beneficial Microorganisms

The discovery by the present inventors reveals the direct correlation between (1) certain “beneficial” bacterial species (e.g., those shown in Table 1a, 4, 6a, 10, 11, or 15), “beneficial” fungal species (e.g., those shown in Table 2, 3a, 5, 7a, or 16), “beneficial” viral species (e.g., those shown in Table 8b), or certain “detrimental” bacterial species (e.g., those shown in Table 1b, 6b, or 10a), “detrimental” fungal species (e.g., those shown in Table 3b or 7b), “detrimental” viral species (e.g., those shown in Table 8a) in an individual's stool or GI tract or in the transfer material derived from donor stool for use in FMT and (2) presence/absence of diseases or significant health benefits conferred by way of FMT treatment to an FMT recipient, such as body weight loss, improved insulin sensitivity, lowered blood cholesterol level, suppression of multidrug-resistant bacteria such as CRE and VRE, alleviation of certain disorders such as aGvHD, UC or CD. This discovery not only allows one to devise an initial screening process to identify appropriate donors and recipients to secure therapeutic efficacy and/or health benefits from the FMT procedure, it also enables different methods for enhancing or optimizing the potential health benefits conferred by the FMT procedure through modulating (increasing or decreasing) the level of one or more of the beneficial or detrimental bacterial, fungal, or viral species shown in the Tables here in a donor stool material and in a recipient prior to the FMT treatment.

As discussed in the above section, when a candidate donor's stool is tested and found to contain an elevated level of one or more of the detrimental bacterial or fungal or viral species such as those shown in Table 1b, 3b, 6b, 7b, 8a, or 10a (e.g., each species is greater than 0.01%, 0.02%, 0.05%, 0.1%, 0.5%, 1%, 1.5%, 2%, 2.5%, 3%, or 3.5% of total bacteria, total fungi, or total viruses, respectively, in the stool sample), the candidate is deemed as unsuitable as an FMT donor, and his stool should not be taken for use in FMT without pre-treatment or modification, as without modification such fecal material is unlikely to yield health benefits such as weight loss, increased insulin sensitivity, or reduced blood cholesterol, elimination of multidrug-resistant bacteria, alleviation of aGvHD, UC, or CD, to the recipient of an FMT treatment. Similarly, when a proposed FMT donor whose stool is tested and found to contain an insufficient level of one or more of the beneficial bacterial or fungal or viral species such as those shown in Table 1a, 2, 3a, 4, 5, 6a, 7a, 8b, 10b, 11, 15, or 16 (e.g., each is less than 0.1%, 0.2%, 0.3%, 0.4%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 11%, 12%, 13%, 14%, or 15% of total bacteria, total fungi, or total viruses in the stool sample), the proposed donor is deemed as an unsuitable donor for FMT intended to confer health benefits such as reduced body weight, sensitized response to insulin, reduced blood cholesterol level, elimination of multidrug-resistant bacteria, and alleviation of aGvHD, UC, or CD, and his fecal material should not be immediately used for FMT due to the lack of prospect of conferring such beneficial health effects unless the stool material is adequately modified. In these cases of expected lack of health benefits from FMT treatment can be readily improved in view of the inventors' discovery, for example, fecal material from a donor can be modified to increase the level of one or more of the beneficial bacterial species (for example, increasing the level of one or more of the bacterial species shown in Table 1a, 4, 6a, 10b, 11, or 15 to greater than 0.1%, 0.2%, 0.4%, 0.6%, 0.8%, 1%, 1.2%, 1.5%, 1.8%, 2%, 2.5%, 3%, 3.5%, or 4% of total bacteria by way of introducing additional amount of such bacteria) and/or to reduce the level of one or more of the detrimental bacterial species (for example, reducing the level of one or more of the bacterial species shown in Table 1b, 6b, or 10a to less than 0.01%, 0.02%, 0.04%, 0.05%, 0.06%, 0.08%, 0.1%, 0.2%, 0.3%, 0.4%, or 0.5% of total bacteria by way of using specific inhibitors of such bacterial species). Pre-treatment schemes with similarly intended goals can be employed to prepare patients who are soon to receive FMT treatment in order to maximize their potential to receive health benefits such as those stated above and herein.

First, for a patient who has been considered for receiving FMT but who has also been deemed an unsuitable recipient of FMT due to an elevated level of one or more of the detrimental bacterial or fungal or viral species shown in Table 1b, 3b, 6b, 7b, 8a, or 10a found in his stool sample, which indicates a diminished chance of a beneficial FMT outcome, measures can be taken to lower the level of such bacteria or fungi or viruses before FMT is commenced so that a much greater chance of achieving beneficial results can be obtained for the FMT procedure. For instance, an anti-bacterial agent capable of suppressing the growth or proliferation of the bacterial species set forth in Table 1b, either generally or specifically, can be administered to the patient in an effective amount such that the level of such detrimental bacterial species in the patient's digestive tract and in the feces is significantly reduced (e.g., each is no more than 0.01%, 0.02%, 0.04%, 0.05%, 0.06%, 0.08%, 0.1%, 0.2%, 0.3%, 0.4%, or 0.5% of total bacteria) prior to the start of the FMT procedure intended for treating obesity/patient weight manipulation or for treating metabolic syndrome or for treating type II diabetes. Similarly, an anti-fungal agent capable of suppressing the growth or proliferation of the fungal species set forth in Table 3b, either generally or specifically, can be administered to the patient in an effective amount such that the level of such detrimental fungal species in the patient's digestive tract and in the feces is significantly reduced (e.g., each is no more than 0.05%, 0.1%, 0.2%, 0.3%, 0.4%, 0.5%, 0.6%, 0.7%, 0.8%, 0.9%, 1%, or 1.5% of total fungi) prior to the start of the FMT procedure intended for treating obesity/patient weight manipulation or for treating metabolic syndrome or for treating type II diabetes.

For the treatment of aGvHD, an anti-bacterial agent capable of suppressing the growth or proliferation of the bacterial species set forth in Table 6b, either generally or specifically, can be administered to the patient in an effective amount such that the level of such detrimental bacterial species in the patient's digestive tract and in the feces is significantly reduced (e.g., each is no more than 0.01%, 0.02%, 0.03%, 0.04%, 0.05%, 0.06%, 0.07%, 0.08%, 0.09%, or 0.1% of total bacteria) prior to the start of the FMT procedure intended for treating aGvHD. Similarly, an anti-fungal agent capable of suppressing the growth or proliferation of the fungal species set forth in Table 7b, either generally or specifically, can be administered to the patient in an effective amount such that the level of such detrimental fungal species in the patient's digestive tract and in the feces is significantly reduced (e.g., each is no more than 0.01%, 0.02%, 0.03%, 0.04%, 0.05%, 0.06%, 0.07%, 0.08%, 0.09%, or 0.1% of total fungi) prior to the start of the FMT procedure intended for treating aGvHD.

For the treatment of ulcerative colitis (UC), an anti-viral agent capable of suppressing the growth or proliferation of the viral species set forth in Table 8a, either generally or specifically, can be administered to the patient in an effective amount such that the level of such detrimental bacterial species in the patient's digestive tract and in the feces is significantly reduced (e.g., each is no more than 0.01%, 0.02%, 0.05%, or 0.1% of total viruses) prior to the start of the FMT procedure intended for treating UC. Similarly, an anti-bacterial agent capable of suppressing the growth or proliferation of the bacterial species set forth in Table 10a, either generally or specifically, can be administered to the patient in an effective amount such that the level of such detrimental bacterial species in the patient's digestive tract and in the feces is significantly reduced (e.g., each is no more than 0.01%, 0.02%, 0.05%, or 0.1% of total bacteria) prior to the start of the FMT procedure intended for treating UC.

For this purpose, the patient's level of detrimental bacterial or fungal or viral species is to be determined at least twice prior to his FMT procedure: once at the initial screening stage, a second time after the initial level is deemed too high for a beneficial FMT outcome and after an anti-bacterial agent has been given to the patient. Once the level of detrimental bacterial or fungal or viral species is confirmed as lowered to a level or percentage that would allow satisfactory FMT outcome, the patient is then ready to undergo FMT treatment.

Second, for a candidate who has been deemed improper to serve as an FMT donor due to a low level in his stool of one or more beneficial bacterial or fungal or viral species such as those shown in Table 1a, 2, 3a, 4, 5, 6a, 7a, 8b, 10b, 11, 15, or 16, he may be disqualified as a donor in favor of anther individual whose stool sample exhibits a more favorable bacterium, fungus, or virus profile. In the alternative, his stool material may still be used, and the expected unsatisfactory FMT outcome can be remedied by supplementing the donor stool material with an effective amount of the beneficial bacterial, or fungal, or viral species. For example, one or more of the bacterial species shown in Table 1a may be introduced from an exogenous source into a donor fecal material so that the level of the bacterial species in the fecal material is increased (e.g., to reach at least 0.1%, 0.2%, 0.3%, 0.4%, 0.5%, 0.6%, 0.7%, 0.8%, 0.9%, 1%, 1.2%, 1.4%, 1.5%, 1.6%, 1.7%, 1.8%, or 2% of total bacteria in the fecal material) before it is processed for use in FMT for the treatment of obesity or metabolic syndrome or type II diabetes. Similarly, one or more of the fungal species shown in Table 2 or 3a may be introduced from an exogenous source into a donor fecal material so that the level of the fungal species in the fecal material is increased (e.g., to reach at least 0.5%, 0.6%, 0.7%, 0.8%, 0.9%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% of total fungi in the fecal material) before it is processed for use in FMT for the treatment of obesity or metabolic syndrome or type II diabetes.

In the case of eradication of multidrug-resistant bacteria such as CRE or VRE, one or more of the bacterial species shown in Table 4 or 15 may be introduced from an exogenous source into a donor fecal material so that the level of the bacterial species in the fecal material is increased (e.g., to reach at least 0.1%, 0.2%, 0.5%, 0.8%, 1%, 1.5%, 2%, 2.5%, or 3% of total bacteria in the fecal material) before it is processed for use in FMT. Similarly, one or more of the fungal species shown in Table 5 or 16 may be introduced from an exogenous source into a donor fecal material so that the level of the fungal species in the fecal material is increased (e.g., to reach at least 0.01%, 0.02%, 0.05%, or 0.1% of total fungi in the fecal material) before it is processed for use in FMT.

In the case of treatment of aGvHD, one or more of the bacterial species shown in Table 6a may be introduced from an exogenous source into a donor fecal material so that the level of the bacterial species in the fecal material is increased (e.g., to reach at least 0.1%, 0.2%, 0.3%, 0.4%, 0.5%, 0.6%, 0.7%, 0.8%, 0.9%, 1%, 1.5%, 2%, 2.5%, 3%, 3.5%, or 4% of total bacteria in the fecal material) before it is processed for use in FMT. Similarly, one or more of the fungal species shown in Table 7a may be introduced from an exogenous source into a donor fecal material so that the level of the fungal species in the fecal material is increased (e.g., to reach at least 0.1%, 0.2%, 0.5%, 0.8%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 11%, 12%, 13%, 14%, or 15% of total fungi in the fecal material) before it is processed for use in FMT.

In the case of treating ulcerative colitis (UC), one or more of the bacterial species shown in Table 10b may be introduced from an exogenous source into a donor fecal material so that the level of the bacterial species in the fecal material is increased (e.g., to reach at least 0.1%, 0.2%, or 0.5% of total bacteria in the fecal material) before it is processed for use in FMT. Similarly, one or more of the viral species shown in Table 8b may be introduced from an exogenous source into a donor fecal material so that the level of the viral species in the fecal material is increased (e.g., to reach at least 0.1%, 0.2%, or 0.5% of total viruses in the fecal material) before it is processed for use in FMT.

In the case of treatment of Crohn's Disease (CD), one or more of the bacterial species shown in Table 11 may be introduced from an exogenous source into a donor fecal material so that the level of the bacterial species in the fecal material is increased (e.g., to reach at least 0.1%, 0.2%, or 0.5% of total bacteria in the fecal material) before it is processed for use in FMT.

Conversely, for a candidate who has been deemed improper to serve as an FMT donor due to a higher level of one or more detrimental bacterial, or fungal, or viral species such as those shown in Table 1b, 3b, 6b, 7b, 8a, or 10a in his stool, he may be disqualified as a donor in favor of anther individual whose stool sample exhibits a more favorable bacterial profile. In the alternative, his stool material may still be used, and the expected unsatisfactory FMT outcome can be remedied by treating the candidate donor or the his stool material with an effective amount of an anti-bacterial, or anti-fungal, or anti-viral agent capable of suppressing the growth or proliferation of such detrimental bacteria or fungi or viruses, respectively, can be administered in a manner essentially the same as the effort described above and herein to suppress or eliminate undesirable bacteria, fungi, or viruses in a recipient's GI tract. Optionally, the stool samples collected from the donor after the treatment can be further improved by artificially adding one or more of the beneficial bacterial/fungal/viral species such as those shown in Table 1a, 2, 3a, 4, 5, 6a, 7a, 8b, 10b, 11, 15, or 16 to reach a substantial level (e.g., each is more than 0.1%, 0.2%, 0.5%, 0.8%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 11%, 12%, 13%, 14%, or 15% of total bacteria, fungi, or viruses, respectively). Since the donor's body, especially the gastrointestinal tract, contains a vast collection of microorganisms many of which are important for the health of gut microflora and for the success of FMT, a useful anti-bacterial/fungal/viral agent for this purpose is preferably not a broad-spectrum agent that kills all bacteria, fungi, or viruses, respectively. Rather, it can be an agent that narrowly and precisely targets the undesirable bacterial, fungal, or viral species without significantly affecting other bacterial, fungal, of viral species, including those that are closely related. Although the agent may be of any chemical compound in nature, small polynucleotides (e.g., siRNAs, miRNAs, miniRNAs, lncRNAs, or antisense DNAs/RNAs) may be the most effective in achieving the specific task of disrupting the expression of one or more key genes in the life cycle of the bacterial, fungal, or viral species being targeted so as to specifically inhibit the proliferation of the target species only without significant impact on other closely related species.

Immediately upon completion of the FMT procedure, the recipient may be further monitored by continuous testing of the level of both beneficial and detrimental bacterial, fungal, or viral species in the stool samples on a daily basis for up to 5 days post-FMT while the clinical symptoms of the condition being treated as well as the intended health benefits are also being monitored in order to assess FMT outcome and the corresponding levels of microorganism in the recipient: in the case of treating obesity/patient weight manipulation, the level of bacterial species set forth in Tables 1a and 1b and the level of fungal species set forth in Tables 2, 3a, and 3b may be monitored in connection with observation of health benefits achieved such as weight loss, insulin sensitivity improvement, and blood cholesterol reduction; in the case of treating conditions caused by multidrug-resistant bacteria such as CRE or VRE and eradication of such harmful bacteria, the level of bacterial species set forth in Table 4 or 15 as well as the level of fungal species set forth in Table 5 or 16 may be monitored in connection with observation of health benefits achieved such as alleviation of the relevant symptoms and suppression or elimination of CRE or VRE; in the case of treating aGvHD, the level of bacterial species set forth in Tables 6a and 6b as well as the level of fungal species set forth in Tables 7a and 7b may be monitored in connection with observation of health benefits achieved such as alleviation of symptoms relevant to aGvHD; in the case of treating ulcerative colitis (UC), the level of bacterial species set forth in Tables 10a and 10b as well as the level of viral species set forth in Tables 8a and 8b may be monitored in connection with observation of health benefits achieved such as alleviation of symptoms relevant to UC; and in the case of treating Crohn's disease (CD), the level of bacterial species set forth in Table 11 may be monitored in connection with observation of health benefits achieved such as allevation of symptoms relevant to CD.

### IV. Kits and Compositions for Improved FMT

The present invention also provides novel kits and compositions that can be used for improving therapeutic efficacy and health benefits delivered by various therapeutic and/or prophylactic treatment schemes involving FMT. For example, in a kit for treating a patient in need of FMT (e.g., for obesity/body weight control, suppression or eradication of multidrug resistant bacterial such as CRE or VRE, alleviation of acute graft-versus-host disease, and alleviation of IBD such as UC or CD), a first composition intended for transplantation into a patient or FMT recipient and a second composition for either (1) increasing the level of one or more of the beneficial bacterial species (such as those shown in Table 1a, 4, 6a, 10b, 11, or 15), fungal species (such as those shown in Table 2, 3a, 5, 7a, or 16), or viral species shown in Table 8b; or (2) reducing the level of one or more of the detrimental bacterial species (such as those shown in Table 1b, 6b, or 10a), fungal species (such as those shown in Table 3b or 7b), or viral species shown in Table 8a—this composition may be in tended to be added to the first composition or it may be intended to be administrated to the recipient, e.g., directly deposited in the GI tract. The first composition comprises a fecal material from a donor, which has been processed, formulated, and packaged to be in an appropriate form in accordance with the delivery means in the FMT procedure, which may be by direct deposit in the recipient's lower gastrointestinal track (e.g., wet or semi-wet form) or by oral ingestion (e.g., frozen dried encapsulated). The second composition in some cases may comprises an adequate or effective amount of one or more of the beneficial bacterial species (such as those shown in Table 1a, 4, 6a, 10b, 11, or 15), fungal species (such as those shown in Table 2, 3a, 5, 7a, or 16), or viral species shown in Table 8b, such that it can be added to the first composition prior to FMT for the purpose of optimizing the prospect of achieving therapeutic efficacy and/or conferring health benefits to the recipient. In other cases, the second composition comprises an anti-bacterial or anti-fungal or anti-viral agent capable of suppressing the growth/proliferation of one or more of the detrimental bacterial species (such as those shown in Table 1b, 6b, or 10a), fungal species (such as those shown in Table 3b or 7b), or viral species shown in Table 8a, which agent may be a broad-spectrum agent that kills bacteria, fungi, or viruses, or a specific inhibitor of the detrimental bacterial or fungal or viral species, as well as one or more pharmaceutically acceptable excipient, such that the composition may be administered to an FMT recipient shortly prior to the procedure, concurrently during the procedure, or immediately following the procedure. The composition is formulated for the intended delivery method of the anti-bacterial, anti-fungal, or anti-viral agent, for example, by injection (intravenous, intraperitoneal, intramuscular, or subcutaneous injection) or by oral ingestion or by local deposit (e.g., suppositories). The first and second compositions are often kept separately in two different containers in the kit. In some cases, both compositions for increasing the beneficial bacterial/fungal/viral species and for suppressing detrimental bacterial/fungal/viral species are present, and they are provided in separate containers as the second and third components of the kit. Typically, the kit will further include printed material providing detailed instructions for users of the kit, such as providing information of the schedule and dosing arrangement for administering the first and second (and optionally third) compositions to a recipient.

In another aspect of this invention, alternative compositions useful in FMT with improved efficacy may be devised to contain at least these two components: (1) a donor stool material containing live fecal microorganisms, and (2) an anti-bacterial agent that specifically suppresses the growth or proliferation of one or more detrimental bacterial species (e.g., those shown in Table 1b, 6b, or 10a) but exhibits no such suppressive or inhibitory effect against other bacterial species, especially those shown in Table 1a, 4, 6a, 10b, 11, or 15; or an anti-fungal agent that specifically suppresses the growth or proliferation of one or more detrimental fungal species (e.g., those shown in Table 3b or 7b) but exhibits no such suppressive or inhibitory effect against other fungal species, especially those shown in Table 2, 3a, 5, 7a, or 16; or an anti-viral agent that specifically suppresses the growth or proliferation of one or more detrimental viral species (e.g., those shown in Table 8a) but exhibits no such suppressive or inhibitory effect against other bacterial species, especially those shown in Table 8b.

Component (2) preferably is not a broad-spectrum bacterium/fungus/virus killing agent; rather, it should be an anti-bacterial/fungal/viral agent specifically targeting the detrimental bacterial/fungal/viral species, respectively (e.g., those shown in Table 1b, 3b, 6b, 7b, 8a, or 10a). For example, it may be short polynucleotide in nature of, e.g., a small inhibitory RNA, microRNA, miniRNA, lncRNA, or an antisense oligonucleotide, that is capable of disrupting the expression of at least one key gene in the life cycle of the targeted detrimental bacterial/fungal/viral species, such that the agent is capable of specifically targeting the bacterial/fungal/viral species only without significantly affecting other closely related bacterial/fungal/viral species. Component (2) is particularly useful in the case of a donor's stool containing a level of one or more of the detrimental bacterial/fungal/viral species too high to permit a satisfactory FMT outcome, as it is capable of locally and specifically suppressing the proliferation of such undesirable bacterial/fungal/viral species so as to ensure the success of FMT despite the less than desirable initial quality of the donor fecal material.

### EXAMPLES

The following examples are provided by way of illustration only and not by way of limitation. Those of skill in the art will readily recognize a variety of non-critical parameters that could be changed or modified to yield essentially the same or similar results.

### INTRODUCTION

The purpose of this invention is to determine if and how donor and recipient fecal bacteria, virome and fungome impact FMT efficacy in the treatment of various human conditions. These conditions include but are not limited to obesity or malnutrition, metabolic diseases, type 2 diabetes mellitus, inflammatory bowel disease, irritable bowel syndrome, antibiotic-resistant infections and graft-versus-host disease. The practical use of the invention includes optimal donor selection, donor/patient stool bacteria, virus and fungi profiling in recipient and donor before and during FMT practice, patient stratification prior to practice of FMT in view of specific bacteria, virome and fungi level/abundance including and up to the species level followed by a sequential treatment of anti-fungal or anti-bacterial prior to FMT therapies on these recipients. Defined “favorable” set of criteria for bacteria, virome and fungi has been provided for establishment for donor stool, donor stool bank and its derived products to optimize diagnostics and therapeutics.

### EXAMPLE 1 OBESITY AND WEIGHT MANIPULATION

### A. Experimental Model for Obesity and Weight Manipulation

### BACKGROUND

The prevalence of obesity is rising at an alarming rate worldwide. In China, over 100 million people are estimated to suffer from diabetes. Similarly, the prevalence of overweight and obese individuals in China has increased by four-fold from 3.7% to 19.0% over two decades. Currently, non-pharmacological interventions based on diet and physical exercise have shown limited long-term success in producing sustained weight loss. Alternatives such as weight loss drugs or bariatric surgery are limited by incomplete resolution of the diseases, side effects, high cost and surgical-related morbidity. Available therapies are insufficiently capable of reducing morbidity and mortality rates from obesity.

Recently, emerging evidence showed that obesity is associated with alterations in the composition of the gut microbiota, and the obese microbiome was more efficient in harvesting energy from the diet. In addition, the bacterial composition differs between lean and obese animals. Therefore, changing the composition of intestinal microbiota has emerged as a novel therapeutic modality to reduce weight and improve insulin sensitivity. In murine and human studies, Bacteroidetes and Firmicutes divisions dominated the microbiota (92.6%) but obese individuals possessed a lower proportion of Bacteroidetes and higher levels of Firmicutes than their lean counterparts. Colonization of germ-free mice with an ‘obese microbiota’ led to a significantly greater increase in total body fat than colonization with a ‘lean microbiota’, suggesting the gut microbiota serves as an additional contributing factor to the pathophysiology of obesity. Obese and lean phenotypes can also be induced in germ-free mice by transfer of fecal microbiota from human donors. Lastly, animal studies have implied a causal relationship as the adverse phenotype can be transferred via fecal transplantation. These data have led to the use of microbiota therapeutics as a potential treatment for metabolic syndrome and obesity.

Fecal microbiota transplantation (FMT) can restore the gut microbial ecology, and has proven to be a breakthrough for the treatment of recurrent Clostridium difficile infection. Furthermore, clinical trials are being conducted to evaluate its use for other conditions including inflammatory bowel disease, irritable bowel syndrome, diabetes mellitus, non-alcoholic steatohepatitis and hepatic encephalopathy. Early results in human have shown that FMT from lean donor when transplanted into subjects with metabolic syndrome resulted in a significant improvement in insulin sensitivity and an increased in intestinal microbial diversity, including a marked increase in butyrate-producing bacterial strains. The therapy is generally well-tolerated and appeared safe. Whether FMT is effective as a treatment for obesity remains to be determined. In addition, FMT from donor to malnourished or underweight individuals may manipulate the microbiota leading to prevention or reversal of weight loss. Efficacy of FMT in obesity and/or malnutrition varies depending on the selection of optimal/favorable donor based on their microbiota profile including but not limited to bacteria, virome or fungi. Compatibility between donor and recipient microbial strains may also be responsible for successful transplant outcomes.

### METHODS

### Human Subjects

Stool samples were obtained from 13 healthy donors (3 females, 7 males and 3 unreported gender participants, age 21-67 years old, body mass index, BMI 18.3-23.0). Thirteen healthy donors included healthy Chinese individuals. These donors were D4, D8, D9, who were FMT donors of a human clinical trial; and Donor 1-9 and Donor 19, who were randomly selected.

### Animal Model and Human-to-Mouse FMT

Age and sex matched C57 mice were put on high fat diets for 4 weeks to establish the obesity model. Obesity model establishment was considered successful if their weight exceeded that of control by 20%. Obese mice were then gavaged with stool from 13 healthy human donor or PBS (control) twice within a week. Their diet was changed from high fat diet to normal diet. Body weight was measured twice a week. Oral Glucose Tolerance Test (OGTT) and Insulin Tolerance Test (ITT) were performed after 2nd FMT. Plasma glucose levels and lipid profiles including total cholesterol (TC), triglyceride (TG), low density lipoprotein cholesterol (LDL-C), and high density lipoprotein cholesterol (HDL-C) were measured at the end of study.

### Fecal DNA Isolation and Metagenomics Sequencing

DNA of fecal samples collected from the 13 donors was extracted and further purified using a DNeasy Blood and Tissue Kit (QIAGEN) according to the manufacturer's protocol. Metagenomics sequencing was performed by first constructing paired-end library with insert size of 350 bp following the manufacturer's instruction (illumina) and sequenced on the NovaSeq Illumina sequencer. Community composition was calculated with MetaPhlan2 using the default settings. Bacterial taxonomy summarization, rarefaction analyses of microbial diversity, compositional differences (dissimilarity value indicated by Unweighted UniFrac Distance) were calculated in R package vegan.

### Findings

Percentage of Body Weight Loss in FMT Group was not Significantly Higher than that in PBS Control when Analysing all Donors Together

Body weight of mice 3 days after second FMT/PBS gavage was compared with body weight of mice prior to first FMT/PBS gavage. Mice were divided into FMT group (n=39) and PBS control group (n=6). Median percentage body weight loss compared to baseline was 13.17% (IQR: 6.08%-18.89%) in FMT group and 11.36% (IQR: 8.43%-15.61%) in PBS control group. A trend towards higher percentage weight loss by FMT compared to control was observed but not significant.

### Effect of Body Weight Loss by FMT Varies According to Donor

To analyse the effect of donor, mice were grouped according to donors. Thirteen groups of mice received FMT from different donors (each group comprised 3 mice), and 1 control group received PBS (6 mice). Eleven groups achieved higher percentage of body weight loss than controls. Mice receiving stool from donor M9 achieved the highest amount of weight loss [mean percentage weight loss 19%; standard deviation (SD) 4.9%]. Although FMT induced weight loss in general, effect varies according to donor. Selection of optimal donor is thus necessary.

Human to Mice FMT was Associated with Improved Insulin Sensitivity in Obese Mice

A total of 60 mice were included in the insulin tolerance test (ITT), 54 received FMT and 6 received PBS. Compared with the PBS control group, mice in the FMT group showed significantly decreased area under curve (AUC) of ITT (P<0.05), which represented an improved ability to clear glucose from the bloodstream. These data showed that FMT improved insulin sensitivity in obese mice.

Blood Serum Total Cholesterol (TC) and Low-Density Lipoprotein (LDL) Level were Significantly Lower in Obese Mice Receiving FMT from Human Stool than Those Receiving PBS

Blood serum TC and LDL level were measured after second FMT/PBS gavage in a total of 33 mice (10 FMT group of 3 mice each receiving FMT from 10 different donors and 1 control group of 3 mice receiving PBS). All FMT group showed a significantly lower TC and LDL level than PBS control group regardless of donor (p<0.001).

### Bacterial Profile of Human Donor Stool Showed Great Variation in Bacteria Composition and Abundance

Comparing between the 13 donors, it was found that the bacterial alpha diversity (Shannon diversity index and Simpson diversity index), and relative abundance of bacteria at phyla level were highly variable.

### “Favourable” and “Unfavourable” Bacteria for Weight Loss

Rate of Body Weight Loss in Obese Mice Showed Significant Correlations with 22 Bacteria

Relative abundance of specific bacteria was calculated form metagenomics sequencing results. To identify specific bacteria associated with donor effect of FMT on weight loss, the relative abundance of specific bacteria were correlated with the mean percentage weight loss observed in each group of mice (n=3) receiving second FMT from the 13 different donors. 5 bacteria were found to have significant positive correlation with mean percentage weight loss, and 17 bacteria were found to have significant negative correlation with mean percentage weight loss.

### Bacteria Criteria for Selecting “Favourable” FMT Donor for Inducing Weight Loss

In an alternative analysis, the 13 donors were classified into “favourable” donors and “unfavourable” donors. “Favourable donors” was defined as donors who were able to induce more weight loss than the mean (13.17%) or median (13.67%) percentage weight loss among the 13 groups of mice, and the rest were defined as “unfavourable donors. Relative abundance of bacteria between the 2 groups were analysed using Linear discriminant analysis (LDA) Effect Size (LEfSe) analysis (linear discriminant analysis effect size>2, adjusted p value<0.05 were considered significant).

The present inventors identified 13 bacteria taxa that were over-represented in “favourable′ donors (favourable bacteria for weight loss) and 7 bacteria taxa that were over-represented in “unfavourable” donors (unfavourable bacteria for weight loss).

A summary combining the “favourable” and “unfavourable” bacteria for weight loss identified above and their relative abundance is shown in Table 1a and Table 1b.

### B. Human Study of FMT for Obesity and Weight Manipulation

### Methods

### Cross-Sectional Study in Human

Stool samples from 23 obese subjects and 15 controls were collected to compare their microbial profile. Obese subjects were defined as having BMI≥28 kg/m², while control subjects were defined as having BMI ≥18.5 kg/m² and <23 kg/m².

### Randomized Controlled Trial of Fecal Microbiota Transplantation (FMT)

Among the obese subjects recruited to the cross-sectional study, 12 subjects who also had type 2 diabetes mellitus were also recruited to a clinical trial titled “A randomised placebo-controlled study of fecal microbiota transplant (FMT) to impact body weight and glycemic control in obese subjects with type 2 diabetes mellitus” (Reference number on ClinicalTrials.gov: NCT03127696). In this study, subjects were randomized into 3 arms: 1) FMT with Lifestyle Modification Program (LMP), 2) FMT alone and 3) Sham with LMP. Primary outcome was defined as 5% weight reduction compared to baseline, which was the weight at randomization before receiving any intervention. Stool samples were collected at baseline. Weight was measured at week 0, 4, 8, 12, 16, 20, 24 and 52.

### FMT Procedures

Volunteers from the general population, spouses or partners, first-degree relatives, other relatives, friends and others who are known or unknown to the subjects were recruited as donors to provide stool for FMT. To ensure donors were healthy and fit for stool donation, donors were first screened with a questionnaire followed by stool and blood tests to rule out any infectious. In addition, only lean donors with BMI <23 kg/m²) were included. Shortly after donor provided stool at the Prince of Wales Hospital, stool were diluted with sterile saline (0.9%). This solution were blended and strained with filter. The resulting supernatant were then stored as frozen FMT solution for future FMT. For subjects randomized to receive FMT, FMT solution from a single donor or mixing of stool from multiple donors were used. For subjects randomized to receive sham, saline solution were infused. During the procedure, 100-200 ml of FMT solution or sterile saline was infused over 2-3 minutes into the distal duodenum or jejunum via OGD.

### Lifestyle Modification Programme (LMP)

Subjects randomized to LMP were seen by a dietitian at week 0, 1, 2, 4, 6, 8 and 12. Dietitian guided and corrected subjects in terms of their dietary habits, physical activity patterns, and other lifestyle habits. Subjects also recorded their dietary history for 7 days before each consultation with the dietitian.

### Fecal DNA Extraction and Metagenomics Sequencing

Fecal DNA was isolated using Maxwell® RSC PureFood GMO and Authentication Kit according to the protocol. DNA libraries were constructed through the processes of end repairing, purification, and PCR amplification. After DNA libraries construction, DNA libraries were sequenced by Illumina Novaseq 6000 with paired-end 150 bp sequencing strategy by Novogene, Beijing, China.

### Read Quality Control and Preprocessing

The raw sequence reads were filtered for quality and adapter removed by Fastp. The human host contaminate reads were remove by Kneaddata with default argument (Reference database: GRCh38 p12). For the metagenomics datasheet, clean reads were processed in Kraken2 by classified to the NCBI Fungi Refseq database (download at Sep 20,2018). Bracken were used to estimate species relative abundance table for downstream analysis.

### Statistical Analysis

Linear discriminant analysis effect size (LEfSe) model was used to identify species having different relative abundance between groups. Only the taxa meeting a LDA threshold value of >2 was considered significant. For species identified by LEfSe model, an alternative analysis was performed to confirm the difference of their relative abundance between groups by Wilcoxon Rank Sum test. α-values <0.05 were considered statistically significant.

### Findings

Relative Abundance of Cercospora beticola and Kazachstania Naganishii were Significantly Higher in Controls than in Obese Subjects

In the cross-sectional study, fecal fungal communities were compared between 23 obese subjects and 15 controls [Median age was 52 (IQR: 47-60) for obese subjects and 57 (IQR: 54-60) for controls; 60% of obese subjects were male and 69.6% of control were male]. By LEfSe analysis, relative abundance of two fungal species, Cercospora beticola and Kazachstania naganishii, was found to be significantly higher in controls than in obese subjects. Median abundance was 0.045 (IQR: 0.038-0.050) in obese subjects and 0.056 (IQR: 0.045-0.063) in controls (p=0.016). Median abundance was 0.004 (IQR: 0.003-0.006) in obese subjects and 0.006 (IQR: 0.004-0.007) in controls (p=0.044). These 2 fungi are favourable fungi for weight loss (Table 2).

Relative Abundance of 4 Fungal Species was Significantly Different Between Subjects Who Achieved Weight Loss and Subjects Who Did not Achieve Weight Loss after Receiving FMT or LMP

From the randomized controlled trial, 12 subjects who had reached week 16 follow up were included in the analysis. To identify fungal species that can predict weight loss, subjects were classified into “weight loss” and “non-weight-loss” group, and their fecal fungal profile at baseline were compared. Subjects were classified into “weight loss” group if their weight loss at 16 weeks was more than 5% compared to baseline, while subjects were classified into “non-weight-loss” group if their weight loss at 16 weeks was less than 5% compared to baseline. Three cases were classified into the “weight loss” group (median percentage weight loss 5.3%) and 9 were classified to the “non-weight-loss” group (median percentage weight loss 0.2%).

By LEfSe analysis, it was found that 1 fungal species, Komagataella phaffii, was significantly higher in “weight-loss” group than in “non-weight-loss” group. High relative abundance of this species in fecal sample at baseline could predict successful subsequent weight loss through intervention of FMT or LMP. Supplementation of this “favourable species” can promote weight loss (Table 3a).

On the other hand, LEFSe analysis also showed that relative abundance of 3 fungal species were identified to be significantly higher in “non-weight loss” group than in “weight-loss” group. These fungal speices were Torulaspora delbrueckii, Encephalitozoon romaleae and Kluyveromyces lactis. High relative abundance of these “unfavourable” species in faecal sample at baseline can predict resistant to weight loss by FMT or LMP (Table 3b).

### Example 2 Multidrug-Resistant Organisms (Carbapenem-Resistant Enterobacteriaceae, CRE and Vancomycin-Resistant Enterococcus, VRE)

### FMT in CRE: Bacterial, and Fungal Determinants of Disease and FMT Outcomes

### Background

Multidrug-resistant organisms (MDRO) present an increasingly serious public health threat to the global community. The prevalence of various MDRO, including carbapenem-resistant Enterobacteriaceae (CRE) and vancomycin-resistant Enterococcus (VRE), has been increasing worldwide, and some have become endemic in certain countries. For example in countries neighbouring Hong Kong, Klebsiella pneumoniae carbapenemase (KPC) and New Delhi metallo-β-lactamase (NDM)-producing Enterobacteriaceae are endemic and widespread in China, while IMP-producing Enterobacteriaceae are widely present in Taiwan and Japan. Data from the Hospital Authority showed that the number of carbapenemase-producing Enterobacteriaceae (CPE) cases increased from 36 in 2012 to 134 in 2015. A large outbreak of VRE involving >200 patients was recently reported in a tertiary hospital in Hong Kong. In some countries, transmission of MDRO is not limited to hospital settings, but become widespread in the community.

The primary site of colonization and persistence of most MDRO is in the gastrointestinal tract. Carriage can persist for months, with up to 40% of individuals still having colonization one year after hospital discharge. Outbreaks of MDRO have been reported in hospitals and long-term care facilities. Around 10% of patients colonized with MDRO would develop clinical infections by the same organism. Infections caused by these MDRO carry significant morbidity and high mortality of up to 50%, partly due to the limited choice of safe and effective antimicrobial therapy. Currently, the control of MDRO transmission is mainly by infection control measures, and there is no proven therapy for eradication of intestinal colonization of MDRO.

There is accumulating evidence showing that the gut microbiota plays an important role in the control of intestinal colonization and infection by pathogenic bacteria. Composition and diversity of gut microbiota in carriers of MDRO were different from non-carriers. Intestinal commensal bacteria help to regulate mucosal innate immunity, e.g., by inducing the production of antimicrobial proteins by epithelial cells. Flagellin has been shown to induce production of RegIIIy, an antimicrobial protein, and reduce VRE colonization in mice. Administration of obligate anaerobic commensal bacteria to mice has also been shown to markedly reduce VRE colonization. Preliminary evidence, mainly from anecdotal reports, have shown that fecal microbiota transplantation (FMT) in human carriers of MDRO were safe and potentially effective in eliminating intestinal colonization by various MDRO, including CRE and VRE, even in immunocompromised patients.

With metagenomics sequencing, the microbial profile of healthy control and FMT recipient was evaluated to identify microbial species associated with CRE colonization. The microbial profile of FMT donor, and microbial profile of recipient pre-and post-FMT were also compared to identify species that are associated with clearance of CRE. These species play an important role in donor or microbial products selection for enhanced FMT efficacy as well as predictive marker for successful CRE clearance after FMT.

### Methods

### Clinical Trial of FMT for Eradication of CRE and VRE in Human

Patients who were age ≥18 years old, had two or more stool or rectal swab positive for CRE or VRE at least one week apart, and did not receive antimicrobial therapy for at least 48 hours prior to infusion of FMT were recruited to a clinical trial. Patients who had active infection of CRE or VRE requiring antimicrobial therapy, pregnancy, active gastrointestinal tract infection or inflammatory disorders, had recent intro-abdominal surgery, had short gut syndrome or use of medications which alter gastrointestinal motility were excluded. CRE was defined as presence of any Enterobacteriaceae with resistance to any of the carbapenems. VRE was defined as presence of Enterococcus species resistant to vancomycin. In this study, patients received 2 FMT using frozen donor stool samples. 100-200 ml of FMT solution or sterile saline were infused over 2-3 minutes into the distal duodenum or jejunum via OGD. Stool samples were collected form patients before and after FMT prospectively.

Human Subjects without CRE or VRE

Stool samples of 2 subjects without CRE or VRE were included as control.

### Faecal DNA Extraction and Metagenomics Sequencing

DNA libraries were constructed through the processes of end repairing, purification, and PCR amplification. Sequencing libraries were prepared using the NexteraXT DNA Library Preparation Kit (Illumina, California, USA), following the manufacturer's guidelines. The sequencing was performed on the NovoSeq PE150 (Novogene, Tianjin, China; standard 2×150 bp run), generating 12G raw data per sample.

### Read Quality Control and Preprocessing

The raw sequence reads were filtered for quality and adapter removed by Fastp. The human host contaminate reads were remove by Kneaddata with default argument (Reference database: GRCh38 p12). For the metagenomics datasheet, clean reads were processed in Kraken2 by classified to the NCBI Fungi Refseq database (download at Sep 20,2018). Bracken were used to estimate species relative abundance table for downstream analysis.

### Statistical Analysis

Linear discriminant analysis effect size (LEfSe) model was used to identify species having different relative abundance between groups. Only the taxa meeting a Linear discriminant analysis (LDA) threshold value of >2 was considered significant.

### Findings

### Fecal Microbiota Transplantation for Eradication of Intestinal Colonization of Carbapenem-Resistant Enterobacteriaceae

Two patients recruited to the clinical trial were successfully cleared from CRE after FMT. Recipient 1 (female, aged 90) had 2 FMT 5 days apart at day 0 and day 5. CRE was tested negative on day 11, and remained negative up till week 5 after first FMT. She then developed foot ulcer infection and received antibiotic therapy at week 6 to week 19 after first FMT. CRE of recipient 1 was tested positive at week 14 and 19. At week 22, it was tested negative again after completion of the 4 courses of Augmentin. Recipient 2 (male, aged 70) received two FMT on consecutive days and all results of CRE follow-up test at week 1, 3, 6 were negative.

Stool samples from recipient 1 and recipient 2 were collected pre- and post-FMT. Both recipient received FMT from the same single donor.

### “Favourable” and “Unfavourable” Bacteria for Selecting FMT Donor in Treating CRE

Bacteria Profile of Patient with CRE was Significantly Different from Healthy Control

In cross-sectional study, fecal bacteria profile was compared between CRE group and healthy control group. CRE group consisted of fecal samples from recipient 1 (R1) and recipient 2 (R2) before receiving FMT, while healthy control group consisted of fecal samples form FMT donor and 2 other healthy controls recruited to recruited to the Hong Kong Health Gut Microbiota Survey.

LEfSe analysis of metagenomics sequencing identified relative abundance of 40 species were significantly different (LDA >2) between CRE group and control group. These represent “favourable” and “unfavourable” bacteria for clearance of CRE.

### Bacteria Profile in Donor and CRE Recipient Pre-and Post-FMT

In the prospective pilot trial, compared to donor, within-subject bacterial α-diversity was consistently lower in both recipients before and after FMT. However, bacterial α-diversity of RI showed increase at Day 18 after FMT and results in R2 showed a greater α-diversity level at day 3 and day 8 as compared with day-14. Relative abundance of individual bacteria species before and after FMT was compared (day-7 vs day 18 in recipient 1, day-3 vs day 18 in recipient 2). For recipient 2, there were 2 time point of fecal samples collected before FMT. Day-3 for recipient 2 was chosen because it was time point closer to FMT. Day 18 was chosen as a post-FMT time point for both recipient because the bacteria profile started to stabilize by this time. The inventors identified bacteria species with increased fold change >1.0 in either recipient 1 or recipient 2. These species presented in very low level or were even not detectable in the pre-FMT fecal samples. Engraftment of these species from donor potentially associate with clearance of CRE. Lachnospiraceae_bacterium_5_1_63FAA showed greatest increase in R1 after FMT and second greatest increase in R2, with fold changes of 1659.5 and 16.9, respectively.

A summary of the “favourable” bacteria for CRE clearance identified above and their relative abundance is shown in Table 4.

### Fecal Fungal Profile in Donor and CRE Recipient Pre-and Post-FMT

In the prospective pilot trial, changes of α-diversity of fungi after FMT were towards an opposite trend in RI and R2, suggesting effect of FMT on fungi diversity were variable in different patients. Relative abundance of individual fungi species before and after FMT was analysed.

Although the short-term donor engraftment with FMT of fungi was not as apparent as bacteria, relative abundance of a few fungi species showed marked changes. The fungi community of recipients following FMT was structurally more similar to the donor profile than that prior to FMT. Fungi species with increased fold change >1.0 in either recipient 1 or recipient 2 were listed below. In particular, Encephalitozoon hellem showed highest fold change in both recipients, increased by 9.31 fold in R1 and increased by 81 fold in R2, respectively. Engraftment and augmentation of these fungal species following FMT could be the determinants of successful CRE decolonization (Table 5).

### EXAMPLE 3 ACUTE GRAFT-VERSUS-HOST DISEASE (AGVHD)

FMT in aGvHD: Bacterial and Fungi Determinants of Disease and FMT Outcomes

### Background

Allogenic hematopoietic stem cell transplant (allo-HSCT) is one of the most promising tumor immunotherapies for hematological disorders. Acute graft-versus-host disease (aGvHD) is a serious complication of allo-HSCT which occurs in 35-45% of recipients, and is associated with 15-25% of mortality. In particular, lower gastrointestinal (GI) tract involvement is complicated by high mortality. Glucocorticoids are used as the first-line therapy for aGVHD, but only about half of the patients respond and no second-line treatment has yet been established. The one year survival rates for aGvHD is less than 30%. So far, there has been no satisfactory improvement in patient survival with refractory GvHD.

Increasing evidence indicates that changes in the gut microbiota composition are closely associated with the occurrence of GvHD. Multiple factors contribute to reduced microbiota diversity in patients having allo-HSCT, including recurrent use of antibiotics, administration of chemotherapy and/or radiation, and altered nutritional patterns. Allogeneic bone marrow transplantation (BMT) lead to a loss of overall diversity and expansion of Lactobacillus and loss of Clostridiales, which increased the risk of subsequent GVHD. Moreover, Enterococci was observed in recipient after allo-HSCT and correlated with the severity of GI-aGVHD. Alteredvirus has also been reported to be linked to enteric GVHD. For instance, an unexpected association of picobirnaviruses was observed with early post-transplant GVHD⁵.

Emerging evidence suggest that altered gut microbiota is associated with acute graft-versus-host disease (aGvHD) in stem cell transplantation (SCT). Fecal microbiota transplantation (FMT) represents a promising treatment for aGVHD. Recently, several small case series have indicated the potential for FMT as treatment for refractory aGVHD. FMT can improve gastrointestinal symptoms, reduce diarrhea and reconstruct gut microbiota in three consecutive patients with refractory GI-aGvHD. Administration of FMT capsules in patient after allo-HSCT decreased the occurrence of aGvHD and increased survival.

To date, there are limited experience of the use FMT in HSCT recipients. Whilst more well-designed clinical studies are warranted to confirm the safety and efficacy of FMT for aGVHD, this life-threatening disease requires immediate attention in some patients. Most studies have focused on restoration of the bacteria diversity, and little is known on the role of phage and fungi in FMT efficacy. In this study, the first teenage case of successful FMT for the treatment of aGvHD (Grade IV) was presented, and serial fecal bacteria, virome and fungi profile was characterized after FMT in the recipient using metagenomics to investigate potential associations between changing microbiome dynamics and the treatment of aGvHD

In a young boy with severe life threatening grade 4 aGVHD of the gut refractory to corticosteroids and biologic therapies, repeated FMT (4 in total) restored the diversity of the microbiome and results in disease improvement. This is the first case of FMT for pediatric GI-GVHD that highlighted restoration of diversity and stable engraftment of bacteria, virus and fungi. Potential donor and recipient bacteria, fungi profile that influences favourable FMT outcomes was identified.

### Case Presentation

A 14-year-old-male who suffered from myelodysplastic syndrome with monosomy 7 underwent HLA-identical sibling allogenic haemotopoietic stem cell transplantation (allo-HSCT) on 21 Dec. 2017. He developed Skin Stage II GvHD (rash 25-50%), gut aGvHD (stage IV, with significant functional impairment), and overall grade IV life threatening GvHD shortly after the allo-HSCT. He had diarrhea, vomiting and abdominal pain. He received the first FMT 77 days after allo-HSCT, followed by three more FMT at 5, 13, and 25 days after the first FMT. The first 3 FMT was from a single donor, D8 and the forth FMT was from a different single donor D4. During the course of four FMTs, patient also received methylprednisolone, cyclosporine A, Infliximab, Ruxolitinib, Entocort, Octreotide, antibiotics, antivirals and antifungals.

On the day prior to receiving first FMT, patient had a stool volume of 465 ml and bowel opening 5 times. On the next day of first FMT, stool volume reduced to 0 while bowel opening reduced to 1 time. On day 5 after first FMT, stool volume increased to 293 ml with blood and watery consistency, while bowel opening increased to two times. The patient received a second FMT from the same donor on day 7. On day 11, stool volume reduced to 100 ml and bowel opening reduced to 1 time. Stool was well-formed, and patient only had mild colicky abdominal pain. Stool volume was maintained at around 100 ml per day and bowel opening was once or none per day thereafter until day 35. Immunosuppressants were slowly weaned off. Patient remained stable until the last follow up at day 117 after first FMT.

### FMT Procedures

The donor stool was obtained from the Stool bank of the Center for Gut Microbiota Research, The Chinese University of Hong Kong.

### Methods

Stool from Donor and Recipient

FMT were administered to the patient on day 0, day 5, day 13 and day 25. Clinical data were in recipient collected daily from day 0 to day 25 when patient had 4 FMT, then monthly until day 117. For this analysis, 1 pre-FMT stool sample, and 3, 4, 6 and 8 stool samples post-1st, 2nd, 3rd and 4th FMT respectively were profiled for bacteria, virus, and fungi by metagenomics sequencing.

Stool samples from the 2 FMT donors, D4 and D8, that were used in the FMT were collected for metagenomics sequencing. Stool samples from donor D8 were collected from 5 different time points within 2 weeks. These samples were labelled as D8-26, D8-27, D8-28 and D8-29. Stool from donor D4 was collected from a single time point and labelled as D4. All these samples were profiled for bacteria, virus and fungi separately. In the 1st and 2nd FMT, stool from donor D8 collected on different days were used. The allocation of stool samples used in the 4 FMT were listed below:
1 ^{st} FMT: D8-26 and D8-27
2^{nd} FMT: D8-28 and D8-29
3^{rd} FMT: D8-34
4^{th} FMT: D4

### Fecal DNA Extraction and Metagenomics Sequencing

Fecal DNA was isolated using Maxwell® RSC PureFood GMO and Authentication Kit according to the protocol. DNA libraries were constructed through the processes of end repairing, purification, and PCR amplification. After DNA libraries construction, libraries were sequenced by Illumina Novaseq 6000 with paired-end 150 bp sequencing strategy by Novogene, Beijing, China. 10-12G raw data for bacteria and fungi metagenomics sequencing.

### Read Quality Control and Preprocessing

The raw sequence reads were filtered for quality and adapter removed by Fastp. The human host contaminate reads were remove by Kneaddata with default argument (Reference database: GRCh38 p12). For the metagenomics datasheet, clean reads were processed in Humann2 for bacterial analysis, and in Kraken2 for fungal and viral analysis. Bacterial taxonomy summarization, rarefaction analyses of microbial diversity, compositional differences (dissimilarity value indicated by Unweighted UniFrac Distance) were calculated in R package vegan.

### Quantitative PCR for Detection of Total Fungal Load in Human Fecal DNA Total

Total fungal loads in human stools were quantified by TaqMan qPCR analysis (Premix Ex TaqTM, TaKaRa) of extracted human fecal DNA using primers36: Fungi-quant-F 5′-GGRAAACTCACCAGGTCCAG-3′; Fungi-quant-R 5′-GSWCTATCCCCAKCACGA-3′, and probe: 5′-TGGTGCATGGCCGTT-3′.

### Statistical Analysis

Linear discriminant analysis effect size (LEfSe) model was used to identify species having different relative abundance between groups. Only the taxa meeting a LDA threshold value of >2 was considered significant. For species identified by LEfSe model, an alternative analysis was performed to confirm the difference of their relative abundance between groups by Wilcoxon Rank Sum test. P-values <0.05 were considered statistically significant.

### Findings

Bacterial Changes after FMT
Alpha-Diversity of Fecal Bacterial Communities in Patient with aGvHD Increased after FMT

Shannon and Simpson index were used to estimate alpha-diversity of the bacterial communities in recipient with aGvHD after FMT (after 1^{st}, 2^{nd}, 3^{rd} and 4^{th} FMTs) and FMT donors (D4 and D8). After receiving the first FMT, alpha-diversity in the stool of recipient increased markedly compared to that before FMT, and after 2^{nd}, 3^{rd} and 4^{th} FMTs the alpha diversity reached a level similar to that of donor D4 and D8.

Restoration of Bacterial Community Structure in aGvHD Patient after FMT

Principal component analysis (PCA) analysis was used to investigate the community structure dissimilarities between the fecal samples of recipient pre-FMT and post-FMT as well as that of donors. After repeated FMTs, the fecal bacteria communities gradually approximate that of the donors, which indicate patient's bacteria community structure was restored and became similar to that of donor.

Seven Bacterial Species Transferred from the Donor Became a Dominant Bacterial Species in FMT Recipient

Engraftment of bacterial species from donor in the patient with aGvHD after FMT was assessed by bar chart. Corynebacterium jeikeium (71.2%) dominated the patient's gut bacterial community and decreased to below 0.1% after the first FMT. Engraftment of 7 bacterial species was observed in the patient after receiving repeated FMT and these species took up a large proportion of the patient's bacterial community. These species were Alistipes onderdonkii, Alistipes putredinis, Clostridium bolteae, Clostridium nexile, Clostridium symbiosum, Eggerthella unclassified, Ruminococcus gnavu.

Relative Abundance of Four Bacterial Species was Significantly Different when the Patient had Diarrhea or No Diarrhea

A total of 22 stool samples were collected pre- and post-FMT from the patient with aGvHD. To identify fecal bacterial that had different relative abundance when patient had diarrhea or no diarrhea, these 22 stool samples were classified according to whether the patient had or did not had diarrhea on the day of stool collection. Diarrhea was defined as having 2 or more bowel opening per day, while non-diarrhea was defined as having 0 or 1 bowel opening per day. LEfSe analysis was used to identify bacterial species which relative abundance were significantly different between stool samples from “diarrhea” or “non-diarrhea” group. Relative abundance of Eubacterium rectale was significantly higher in diarrhea group, while relative abundance of Alistipes putredinis, Alistipes onderdonkii and Clostridium hathewayi were significantly higher in non-diarrhea group. “Favourable” donor should contain a high relative abundance of Alistipes putredinis, Alistipes onderdonkii and Clostridium hathewayi and low relative abundance of Eubacterium rectale.

aGvHD Symptoms Significantly Correlated with 5 Bacterial Species

To identify specific bacterial associated with treatment outcome, relative abundance of specific bacterial was correlated with the GvHD symptoms including number of bowel opening, stool volume and volume of vomiting by linear regression. One bacterium was found with significant positive correlation with number of bowel opening and stool volume. “Favourable” donor should contain a low relative abundance or absences of Corynebacterium jeikeium. On the other hand, 4 bacterial species showed significant negative correlation with vomiting symptom. “Favourable” donor should contain a high relative abundance of these bacterial (listed below): Alistipes putredinis; Clostridium bolteae; Clostridium hathewayi; Clostridium nexile.

A summary combining the “favourable” and “unfavourable” bacteria for treating aGvHD identified above and their relative abundance is shown in Table 6a and Table 6b.

Fungal Changes after FMT
Increased Stool Diversity of Fungal Species in aGvHD after FMT

Shannon and Simpson index were used to estimate alpha-diversity of the fungal communities in recipient with aGvHD after FMT and FMT donor (donor 4 and donor 8). After receiving the first FMT, alpha-diversity increased markedly compared to that of before FMT. Alpha-diversity of fungal species continued to increase steadily after the subsequent three FMTs and reached a level similar to that of donors D4 and D8.

Increased Total Fungal Load in aGvHD Patient after FMT

Total fungal load was measured by qPCR. Total fungal load in the patient increased to a level similar to that of donor after receiving FMT from donor D8, then decreased again after a few days and remained low after receiving second FMT. The level in the patient increased again after receiving the third FMT. Total fungal load decreased slightly after fourth FMT using stool from donor D4 who had a slightly lower total fungal load than donor D8.

Relative Abundance of 2 Fungal Species Significantly Decreased after FMT while 12 Fungal Species Transferred from Donor Became a Dominant Fungal Species in FMT Recipient

Engraftment of fungi species from donor in the patient with aGvHD after FMT was assessed by bar chart. Fusarium oxysporum (61.44%) and Botrytis cinereal (29.13%) dominated the patient's gut fungal community, and decreased significantly after the first FMT. These are considered as “unfavourable” fungi. Twelve species engrafted in the patient after receiving repeated FMT and took up a large proportion of the patient's fungal community. These species were Schizosaccharomyces pombe, Eremothecium sinecaudum, Cercospora beticola, Fusarium venenatum, Thermothelomyces thermophila, Colletotrichum higginsianu, Fusarium verticillioides, Thielavia terrestris, Candida dubliniensis, Eremothecium gossypii Fusarium pseudograminearum, and Neurospora crassa. These are considered as “favourable” fungi.

aGvHD Symptoms Significantly Correlated with 21 Fungal Species

To identify specific fungi associated with treatment outcome, the relative abundance of specific fungi was correlated with the GvHD symptoms including number of bowel opening, stool volume, and volume of vomiting by linear regression. Two fungi were found to show significant positive correlation with number of bowel opening and stool volume. Based on these findings, “favourable” donor should contain a low relative abundance of these two fungi, Fusarium oxysporum and Botrytis cinerea. On the other hand, 19 fungi showed significant negative correlation with at least one of the symptoms. “Favourable” donor should contain a high relative abundance of these fungi.

A summary combining the “favourable” and “unfavourable” fungi for treating aGvHD identified above and their relative abundance is shown in Table 7a and Table 7b.

### EXAMPLE 4 INFLAMMATORY BOWEL DISEASE VIROME

### Mucosal Virome in Healthy Population and Ulcerative Colitis

### Background

Ulcerative colitis (UC), one subtype of inflammatory bowel disease (IBD), is a remitting and relapsing inflammatory disease affecting the entire large intestine, which usually begins in the rectum and spreads upwards. The incidence of UC is continuing to increase especially in developing nations as well as the newly industrialized countries. While the etiology of IBD remains ambiguous, IBD is hypothesized to arise from aggravated immune responses towards the gut microbiota in genetically susceptible individuals. Fecal virome alterations in IBD have been investigated both in humans and in mice, characterized by a bloom in bacteriophages from the order Caudovirales. However, it is unclear of the mucosal virome composition and function either in health or in IBD, meanwhile the knowledge on mucosal bacteria alterations in UC is also limited. To begin to address these conundrums, the present inventors enriched the virus-like particles (VLPs) from the rectum of healthy individuals and patients with UC, performed ultra-deep vriome metagenomic sequencing and 16S rDNA sequencing to determine mucosal virome, bacteriome and viruses-bacteria interactions in subjects with UC compared to healthy controls. This study is the first and largest to date to characterize the mucosal virome in health and UC.

### Methods

### Study Subjects

48 healthy subjects versus 63 patients with UC from Hong Kong, 20 healthy subjects versus 20 patients with UC from Beijing, and 8 healthy subjects versus 8 patients with UC from Xiangshan, Zhejiang Province (all Chinese), were recruited into this study, with informed consent. Patient inclusion criteria include subjects aged ≥18 with a diagnosis of UC defined by endoscopy, radiology and histology. Controls comprised of individuals undergoing colonoscopy for polyp or colorectal cancer screening, or investigations of gastrointestinal symptoms, and friends and spouses or partners of patients at local hospitals, or any individuals who are interested to participate in this study. Rectal biopsies from the study subjects were collected via endoscopy and then stored at −80° C. for downstream mucosal virome and bacterium analysis.

### Virus-Like Particles (VLPs) Enrichment and Sequencing

Virus-like Particles (VLPs) were enriched from the rectal biopsies of patients with UC and healthy subjects, using a modified protocol according to previously described methods. The Qualified libraries were amplified on cBot to generate the cluster on the flowcell (TruSeq PE Cluster Kit V3-cBot-HS, Illumina). The amplified flowcell were sequenced pair end on the HiSeq Xten System (TruSeq SBS KIT-HS V3, Illumina) (BGI, Shenzhen, China; standard 2×150 bp run), generating 20-60 million raw sequences (5-8G raw data) per sample.

### Sequence Processing and Quality Control

Raw reads were filtered by SOAPnuke (v 1.5.3) (website: soap.genomics.org.cn/) developed by BGI as follows: (i) adaptors removed, (ii) read removed if N base is more than 3% of the read, (iii) read removed if bases with quality low than 20 were more than 40% of read, (iv) all duplicates removed. Human sequences were removed from the quality-trimmed dataset by DeconSeq (v 0.4.3) with default parameters and the human reference GRCh38.

### De Novo Contig Assembly and Taxonomy Annotation

Contigs were assembled using the IDBA (v 1.1.1), using maximum kmer length 120, with a minimum contig length of 1,000 bp. The assembled contigs were clustered at a 95% identity level using CD-HIT to generate a unique contig consortium.

### Virome Abundance Data Analysis

To estimate contig abundance and calculate sequence diversity, all reads were aligned to the resulting curated contigs using Bowtie2 (v 2.2.9). The mapped sequence counts, contig lengths, and total sequence counts were used to normalize the sequence counts and represent the RPKM (reads per kilobase per million) of each sample to the contigs. These values were used to generate viral abundance tables at various taxonomy levels.

The virome abundance data was imported into R 3.2.3. Diversity, evenness, and richness calculation were performed using phyloseq in R. Spearman correlation and their significance were calculated using the cor and cor.test functions in R, respectively. For the viruses-bacteria correlations in a diversity and taxa abundance, Spearman correlations were calculated. Correlation plots were generated using the corrplot R package. Heat maps were generated using the pheatmap R package.

### Non-Metric Multidimensional Scaling Analysis

The difference in mucosal viral community structures between controls and UC was performed via NMDS (Non-metric multidimensional scaling analysis) plot based upon Bray-Curtis dissimilarities among all subjects.

### DESeq, Random Forest and LEfSe Linear Discriminant Analysis

To compare differences in the configurations of mucosal virome and bacterial microbiome as well as the functions of virome between UC patients and healthy household controls, between mucosal virome enterotypes, Differential analysis were performed. DESeq and Random Forest were performed in R via DEseq and randomForest package respectively. Lefse analyses were performed on the Huttenhower lab Galaxy server (website: huttenhower.sph.harvard.edu/galaxy/). DB-RDA analysis was also performed in R to delineate the effect of medication and health on mucosal virome configutation.

### Virome Function Analysis

Virome functions were annotated via HUMANN2 v0.9.4. Predicted functions were collapsed by Gene ontology terms and Pfam protein family identies, with abundance values expressed in RPK (reads per kilobase). To establish the presence or absence a function within a sample, a stringent RPK threshold value >10 was used to define as present.

### Mucosal Bacterial DNA Extraction

Bacterial DNA was extracted from rectal biopsies using Maxwell RSC Tissue DNA kit (Promega, Madison, Wisconsin) according to the protocal.

16S rRNA Sequencing and Quality Control

The final fecal DNA samples were sequenced on the Illumina Hiseq 2500 platform (V4 region, 2×250 bp). Quality control and data analysis were implemented in mothur (v 1.38.0) as previously described. Any sequences with ambiguous bases and anything longer than 275 bp were removed, and aligned against the non-redundant Greengenes database (v 13.8) using the NAST algorithm.

16S rRNA Sequencing Data Analysis

The resulting sequences were classified against the Greengenes database and annotated with deepest level taxa represented by pseudo-bootstrap confidence scores of at least 80% averaged over 1,000 iterations of the naive Bayesian classifier.

### Findings

### Alterations in the Diversity of Mucosal Virome in UC

The rectal virome of 63 patients with UC was compared with that of 48 healthy subjects in Hong Kong. On average, 56,632,558±14,330,713 clean paired-end reads were obtained from the enriched rectal VLP preparations. The mucosal virome composition was investigated at the order, genus and species levels in health and UC.

Compared with healthy individuals, UC subjects had a significantly higher abundance of viruses in the mucosa (Mann-Whitney test, p=0.017) and decreased viral diversity, evenness and richness (t test, p=0.013, 0.020, and 0.029, respectively). The alterations in Caudovirales bacteriophages, predominant mucosal viruses were next explored, and it was found that Caudovirales abundance also expanded in UC (Mann-Whitney test, p=0.003), while the species diversity, evenness and richness within the Caudovirales order were all decreased (t test, p=0.009, 0.017, and 0.018, respectively). Collectively, these findings indicate dysbiosis of the mucosal virome in patients with UC.

There were more total viruses and Caudovirales bacteriophages enriched in inflamed mucosa than in non-inflamed mucosa among UC patients (Mann-Whitney test, p=0.015 and 0.017). In comparison with the a diversity differences in the mucosal virome of non-inflamed mucosa of UC patients versus that of healthy controls, the decreases in the diversity, evenness and richness of the total viruses and Caudovirales bacteriophages in the inflamed mucosa versus that of healthy controls were statistically more pronounced. Thus, it suggests that gut inflammation is a significant contributor to mucosal virome dysbiosis in UC.

Different Mucosal Virome Configurations Between Healthy Population and Patients with UC

At the family level, Microviridae (single-stranded DNA phage), Mycoviridae, Podoviridae (double-stranded DNA phages from the Caudovirales order) and Penumoviridae (Eukaryotic virus) were more abundant in UC than in controls, whereas Anelloviridae (Eukatyotic virus) were higher in controls than in UC. At the genus level, Phix174microvirus, Plvirus, Lambdavirus, T4virus, P22virus (all Caudovirales bacteriophages) and Orthopneuovirus were enriched in UC, whereas giant viruses Coccolithovirus, Minivirus and Vertebrate-infecting virus Orthopoxvirus (all Eukaryotic viruses) were enriched in controls. In congruent with these observations, more Caudovirales bacteriophage species expanded in abundance in UC mucosa relative to healthy subject mucosa, including Escherichia phage and Enterobacteria phage. Altogether, these findings indicate that dysbiosis in prokaryotic viruses, particularly Caudovirales bacteriophages, are prominent in UC mucosa, while some eukaryotic viruses are more abundant in healthy controls, indicating an association between bacteria dysbiosis and bacteriophage expansion in UC and an association between eukaryotic virus infection and host immunity maturation in health which may protect the host from autoimmune diseases.

Exploiting Random Forest, the inventors performed classification on the mucosal viromes in the Bejing and Xiangshan cohort. All the subjects were classified as Enterotype 1. Differential analysis on all Enterotype 1 subjects between healthy controls and UC in Hong Kong cohort identified a panel of disparate viral species. However, only two species were replicated in the validation cohort, algea-infecting Feldmannia species virus, significantly highly present in UC Enterotype 1 subjects relative to control Enterotype 1 subjects in Xiangshan cohort, and Pseudomonas virus, significantly highly present in control Enterotype 1 subjects relative to UC Enterotype 1 subjects in Beijing cohort. All together, it indicates that there is a significant geographical effect on the mucosal virome structure, resulting in a large variation in the mucosal virome composition between cohorts.

Functional alterations in mucosal virome in UC

The mucosal virome functions were then compared between health and UC.

Though the most abundant functions did not differ in abundance between health and UC, healthy individuals showed a richer virome function, in terms of both GO and Pfam protein functions, whereas UC subjects exhibited a significant abolishment of functions. Nonetheless, several molecular functions were determined to be more abundant in UC than in healthy mucosal virome, including DNA template negative regulati on of transcription, beta-lactamase, glutamine amidotransferase, glycosal hydrolases, Type II/IV secretion system and multicopper oxidase, all of which were linked to phage lysis of bacteria host as well as bacteria functions. This result implies that the enriched mucosal viral functions in UC are associated with bacteria fitness, pathogenicity and antibiotics resistance, and that an intensive genetic swap between bacteria and bacteriophages may exist in UC mucosa.

### Mucosal Bacteriome Alterations and Trans-Kingdom Interactions Between Virome and Bacteriome at Mucosa

the bacterial microbiome alteration in UC mucosa was further assessed compared to healthy control mucosa. Bacterial diversity and richness were significantly increased (t test, p=0.024 and 0.007, respectively, indicating an expansion of diverse bacteria presence in the mucosa of UC patients. UC mucosal bacteriome showed distinctive structure to that of controls, at the phylum, family and genus levels. Quantitative differential analysis identified a number of bacterial taxa distinguished between health and UC mucosa. Among them, Firmicutes (phylum), Pseudomonadaceae, Ruminococcaceae, Thermaceae, unclassified Clostridiales, Vellonellaceae (family), and Coprococcus (genus) were significantly enriched in UC than in controls.

To characterize the relationship between bacteriome and virome in the mucosa, the correlation between the a diversity (diversity, evenness and richness) of the bacteriome and that of the virome was evaluated. In controls, there were significant intra-kingdom a diversity correlations. However, the significances of within-bacteria-kingdom a diversity correlations observed in controls were lost in UC, indicating a more dysbiotic state of the mucosal bacterial microbiota than the dysbiosis of the mucosal virobiota in UC.

### EXAMPLE 5 CROHN'S DISEASE AND ADHERENT INVASIVE E. COLI (AIEC)

### FMT in Crohn's Disease: Presence of AIEC and FMT Outcome

### Background

Crohn's disease (CD), one of the two main inflammatory bowel diseases (IBD), is a life-long disabling condition of the gut. In Hong Kong, the incidence of Crohn's disease has risen by 7-fold in the past decade. The cause of Crohn's disease is unknown. While genetic factors may play a role, strong evidence suggests that the bacteria in the gut are likely to be fundamental to the development of disease. FMT is currently being explored as a potential therapy for IBD. However, the mechanisms and factors affecting treatment outcome is unclear. Adherent invasive Escherichia Coli (AIEC) can invade the gut epithelium and colonise the mucosa of IBD patients. They can also survive inside macrophages without inducing cell death, allowing them to lurk in the mucosa and cause damage in the long term. Since FMT directly changes the content of the gut lumen, we hypothesize that FMT may not be able to eradicate the AIEC bacteria residing inside the epithelial cells and macrophages could continue to cause inflammation. Therefore, the aim of our study is to determine whether the presence of AIEC strains could stultify the efficacy of FMT and worsen the outcome of the disease.

### Method

Study subjects

Patients with ileal Crohn's disease location L1-ileal or L3-ileocolonic according to Montreal classification) disease were recruited in Hong Kong. Patients were aged ≥18 with a diagnosis of ileal Crohn's disease defined by endoscopy, radiology and histology (ileal or ileocolonic). Subjects without IBD were recruited as controls. These comprised of individuals aged ≥18 who would undergo colonoscopy for polyp or colorectal cancer screening, or investigations of gastrointestinal symptoms, and friends and spouses or partners of patients at local hospitals, or any individuals who were interested to participate in this study. Clinical data were collected using a standardized data collection form and ileal biopsies were collected during ileocolonoscopy. Biopsies were then stored at at −80° C. for downstream analysis. Subjects were excluded if they had taken antibiotics, probiotics or prebiotics within 3 months of biopsies taking. All subjects consented to participate.

Quantification of Mucosal E. coli Level and Isolation of E. coli Strains that Colonized the Ileal Mucosa

The biopsy samples were thawed and weighed in order to calculate the isolated bacteria to the weight of the tissues. The bacteria associated with the mucosal surface Intestinal mucosa adherent flora was isolated by gentle shaking for 5 minutes in PBS. Internalized bacteria was released by treating the biopsies with Triton 1X and Ultra-Turrax. The isolated bacteria were cultured at 37° C. overnight. The colony forming units (CFUs) were counted on the next day to quantify mucosal associated E. coli. The mucosal E. coli level was calculated by dividing the number of colony forming units by the weight of mucosa biopsies.

For each patient, 48 bacterial colonies from mucosal associated E. coli isolated on Drigalski medium and positive for lactose metabolism (E. coli differential character) were cultured in 96-well microplates in Luria-Bertani medium supplemented with 15% glycerol and stored at −80° C.

### Determination of AIEC Positivity

AIEC positivity representing the presence of AIEC was determined by first isolating E. coli strains in the mucosal biopsies (as described above), followed by screening for main characteristics of AIEC strains in the isolated strains and classification based on these characteristics of AIEC. Main characteristics of AIEC include adhesion, invasion to intestinal epithelial cells and survival within macrophages. E. coli strains having an invasion index greater than or equal to 0.1 relative to the initial inoculum and having the ability to survive and multiply within macrophages at 24 hours post-infection (% survival in macrophages greater than 100) were considered as AIEC.

### Bacteria Administration and Fecal Microbiota Transplantation in DSS Colitis Mouse Model

8-9 weeks old male C57B/L6 wild type mice (n=50) were used to construct acute DSS colitis mouse model to investigate the effect of FMT on AIEC infected mice. A group of mice was given only clean drinking water as control (n=6). 44 mice were subjected to drinking water supplemented with 2% DSS.

Among 21 AIEC strains isolated from CD patients, one with the strongest invasion ability, AIEC 62d, was selected for animal experiment. Mice were gavaged with a dose of 10⁹ CFU of this AIEC strain (n=22) or a non-pathogenic E. coli strain, K12 (n=22), for three consecutive days, starting from the first day of DSS treatment.

Stool were collected from healthy mice and diluted 1:10 with sterile PBS. After sedimentation, 200 μl of the supernatant were gavaged to each mouse for three consecutive days as FMT treatment. Mice from AIEC group and K12 group (n=6, each) were sacrificed on day 7 as “before FMT” group. The remaining 32 mice were then gavaged with fecal matter from healthy mice or sterile PBS (n=8, each) for three consecutive days starting from day 7.

### Quantification of Fecal AIEC in Mice

Numeration was done by platting PBS-resuspended feces on LB agar plates containing ampicillin (50 μg/mL) and erythromycin (25 μg/mL) since the AIEC strain is resistant to these antibiotics, and no such resistant bacteria were found in non-infected mice. On the next day, colony forming units (CFU) were counted and the number of CFU per mg of feces was evaluated.

### Assessment of Severity of Colitis in Mice

Bodyweight of the mice were measured daily. Stool samples were collected every two days. Colon length and histology score were measured at sacrifice to evaluate the severity of colitis. Colon length was measured from cecum to rectum. Blinded histologic scoring was performed on H & E stained colonic tissue as follows. Each section was assigned four scores based on the degree of epithelial damage and inflammatory infiltration into the mucosa, submucosa and muscularis/serosa, resulting in a total scoring range of 0-12 per mouse. The average scores for control and DSS-treated groups were then tabulated.

DNA Extraction and 16s rRNA Sequencing of Mice Specimens

The proximal colon tissue DNA was extracted to assess the mucosal associated microbiota. Stool and tissue from mice sacrificed on day 7 were characterized as “before FMT,” whereas stool and tissue harvested on day 14 were characterized as “after FMT.” DNA was extracted using Maxwell® 16 Tissue DNA Purification Kit following manufacturer's instruction. The extracted DNA were send to Novogene (HK) company limited for library construction and V3-V4 region of 16s rRNA sequencing on Illumina PE250 platform.

### Bioinformatics Analysis

The conversion from raw data to OUT tables were done using Mothur pipline. Sequences were classified by 97% resemblance.

### Findings

Mucosal E. coli was Present in Higher Level in AIEC Positive CD Patients than AIEC Negative CD Patient and Healthy Control

It has been reported that E. coli bacteria are over-represented at the mucosa of CD patients when compared to healthy controls. To assess whether E. Coli bacteria are further over-represented at the mucosa of CD patients with AIEC, the total E. coli load was measured in ileal mucosa taken from 56 CD subjects with ileal involvement and 24 healthy controls.

Among all the subjects we examined, 37.5% (n=21) of CD patients and 8.3% (n=2) of healthy controls were AIEC positive. The total E. coli load of AIEC positive patients was significantly higher than that of AIEC negative patients and healthy control (p=0.0323 and p<0.001 after logarithmic transformation, Turkey's multiple comparisons test).

Fecal AIEC Level in Mice was Only Transiently Reduced after FMT

Effect of FMT on AIEC level was investigated in DSS colitis mice infected with AIEC. Changes of AIEC load in mice before and after FMT was evaluated in fecal samples. In AIEC mice treated with FMT, the fecal AIEC load decreased immediately after mice was treated with FMT for 3 consecutive days but gradually increased subsequently. Comparing AIEC mice with and without FMT, the fecal AIEC load on day 9 and day 12 after AIEC infection in FMT group was significantly lower than that of no FMT group. However, on day 14 after AIEC infection, the difference of AIEC load between FMT group and no FMT group became insignificant. These data indicate that FMT alone may not be enough to eradicate AIEC in mice. Treatment that specifically target AIEC should be incorporated to eradicate persistent AIEC infection.

### FMT Efficacy in Treatment of Colitis was Compromised by AIEC Infection

The effect of AIEC on FMT efficacy in the treatment of colitis was then investigated. Effect of FMT was compared between mice infected with AIEC and mice infected with K12, a non-pathogenic E. coli strain. Severity of colitis was assessed by body weight, colon length, and histological score.

For body weight, the percentage recovery of body weight after FMT compared to baseline (day 0) was evaluated, in K12 infected mice, percentage of body weight recovery at day 12 was higher in mice treated with FMT than that without FMT (99% vs 95%, p=0.0243, 2-way ANOVA). On the other hand, for AIEC infected mice, body weight of mice with or without FMT treatment showed no significant difference at day 14 (p=0.98, 2-way ANOVA). Comparing body weight recovery after FMT at day 14, body weight of K12 infected mice recovered to 97% of initial body weight, while body weight of mice infected with AIEC only recovered to 91% (p=0.048, 2-way ANOVA).

For colon length, in mice infected with AIEC, colon length was not significantly different between group of before FMT and after FMT (p=0.88, Kruska-Wallis test with multiple comparisons). Conversely, colon length of K12 infected mice significantly increased after FMT (mean colon length=SEM 6.80±0.12 mm before FMT vs 8.02±0.27 mm after FMT, p=0.0042, unpaired t test).

For histological score, after FMT, there was less improvement in histological score in AIEC infected mice, compared with mice infected with K12 (2.0 vs 3.0, p=0.086, Mann-Whitney test), indicating less recovery of colitis in AIEC infected mice. These results show that FMT ameliorated DSS induced colitis in AIEC negative (K12 infected) mice, but its efficacy was compromised in the presence of AIEC.

Absence of Favourable Bacteria in AIEC Infected Mice Despite Treatment with FMT

The mucosal and fecal microbiota of the aforementioned AIEC infected and K12 infected mice were analyzed before and after FMT. The presence of AIEC in the mouse gut caused reduced bacterial diversity in mice fecal microbiota. FMT treatment increased the alpha diversity of K12 infected mice, but not AIEC infected mice. LEfSe analysis revealed several taxa that successfully engrafted K12 mice, but failed to colonise in AIEC infected mice. Most of these taxa belong to the Firmicutes family, which resembled the feature of the mucosa microbiota of IBD patients, indicating AIEC as a driver in dysbiosis in the mouse mucosa. These data indicate a casual role of AIEC in the pathogenesis of IBD. These taxa are listed in Table 11.

Presence of AIEC might be an impediment in FMT efficacy. The prevention of colonization of potentially beneficial commensals might be a mechanism for AIEC to affect FMT efficacy.

### EXAMPLE 6 OBESITY

### Methods

### Open-Label Human Study of FMT for Obesity and Weight Manipulation

An open-label clinical trial of fecal microbiota transplant (NCT03789461) in obese subjects was conducted. Subjects aged 18-75, has a body mass index (BMI) ≥28 kg/m² and <45 kg/m² and with informed consent obtained were recruited. During this study, subjects received intensive FMT for a total of 20 days. Every week during treatment period, subject received 5 days of FMT (5 days on and 2 days off). During the same period, subjects also received dietary and lifestyle advice.

FMT: Stool were obtained from volunteers from general population including spouses or partners, first-degree relatives, other relatives, friends and others who are known or unknown to the recipients. Prior to stool donation, volunteers were screened by questionnaire and laboratory test. A series of laboratory tests for infectious diseases and interviews were done. Stool from the eligible donors were used in this study. Prior to receiving FMT, subjects received 5 days of antibiotics consisting of Vancomycin 500 mg 3 times daily, Metronidazole 500 mg 3 times daily and Amoxicilin 500 mg 3 times daily to enhance the engraftment of the microbiota from FMT. Then subjects received 20 days of FMT. 100-200 ml of FMT solution were infused to patients via standard procedures including Oesophago-gastro-duodenoscopy (OGD), sigmoidoscopy, or enema in either in-patient or out-patient settings.

1. Via OGD: 100-200 ml of FMT solution was infused over 2-3 minutes into the distal duodenum or jejunum via OGD. After infusion, subjects were monitored for 1 hour before discharge.

2. Via Sigmoidoscopy: 100-200 ml of FMT solution was infused over 2-3 minutes into the distal colon via sigmoidoscopy. After infusion, subjects were monitored for 1 hour before discharged.

3. Via Enema: 100-150 ml of FMT solution was self-administered or with help from research team via enema. Subjects were instructed to retain the enema for 20-30 minutes. 4 mg loperamide was given before each enema to enhance the retention of FMT solution.

### Fecal DNA Isolation and Metagenomics Sequencing

DNA of faecal samples collected from the donors was extracted and further purified using a DNeasy Blood and Tissue Kit (QIAGEN) according to the manufacturer's protocol. Metagenomics sequencing was performed by first constructing paired-end library with insert size of 350 bp following the manufacturer's instruction (illumina) and sequenced on the NovaSeq Illumina sequencer. Community composition was calculated with MetaPhlan2 using the default settings. Bacterial taxonomy summarization, rarefaction analyses of microbial diversity, compositional differences (dissimilarity value indicated by Unweighted UniFrac Distance) were calculated in R package vegan.

### Findings

Body weight changes of obese subjects up to week 8 were analyzed. Five consecutive subjects received FMT from 3 different donors. Fecal bacteriome of these donors were profiled through metagenomics sequencing. Donor 15 and Donor 16 has 2 and 3 favourable species for weight loss (Table 12) of relative abundance of >0.1; and 2 unfavourable species for weight loss (Table 13) of relative abundance≤0.01%. In contrast, Donor 8 has none of these favourable bacteria that Donor 15 and Donor 16 have, and relative abundance of the 2 unfavourable species were >0.01% (Table 12 and Table 13). Based on these profile, Donor 8 was considered as unfavorable donor for weight loss while Donors 15 and 16 were considered as favourable donor for weight loss. At week 8, subjects receiving FMT from Donors 15 and 16 had more weight loss than recipients of donor 8 (Table 14).

### EXAMPLE 7 OBESITY AND LIPID METABOLISM

### Experimental Model for Obesity and Lipid Metabolism

### Bacteria and Media

### Sutterella Wadsworthensis Growth Conditions

Sutterella wadsworthensis (SW) were obtained from DSMZ, the liquid growth medium was without glucose, supplemented with molasses at a concentration corresponding to 2% sucrose; sodium lactate (Chempur Poland) 7.41 g/L; sodium acetate (Chempur Poland) 7 g/L; and 0.2% yeast extract (BD Bioscences USA) in different combinations. Starting pH of all media was 7.0. Cultures were incubated in a micro-aerophilic atmosphere, comprising of 5.9% oxygen, 7.2% carbon dioxide, 3.6% hydrogen and 83.3% nitrogen at 37° C. This atmosphere was generated using Anoxomat® Atmosphere Generating System, from Mart® Microbiology b.v. (9200 JB Drachten, Netherlands). Chopped meat carbohydrate medium (CM) were purchased from BD. Cultures were grown under anaerobic conditions (Coy Laboratory Products, 75% N₂, 20% CO₂, 5% H₂) in 37° C. without shaking. Plates were reviewed twice weekly for up to one month. Any bacterial isolate deemed Gram-negative and oxygen sensitive (by virtue of failed subculture in room air) was identified by sequencing of the 16S rRNA gene and sequence search on NCBI BLAST.

Roseburia intestinalis Growth Conditions

Roseburia intestinalis (RI) were purchased from DSMZ collection, DSM-13018 Media were prepared and maintained anaerobically using-free Oxygen device. The isolates were routinely maintained by growing for 16-18 h at 37° C. in 7±5 ml aliquots of M2GSC medium. Substrate utilization and hydrolysis. The Medium consisted of (in 100 ml) 1 g casitone, 0±2.5 g yeast Extract, 0±4 g NaHCO₃, 0±1 g cysteine, 0±0.45 g K₂HPO₄, 0±0.45 g KH₂PO₄, 0±09 g NaCl, 0±009 g MgSO4, 0=009 g CaCl₂), 0±1 mg resazurin, 1 mg haemin, 1 μg biotin, 1 μg cobalamin, 3 μg p-aminobenzoic acid, 5 μg folic acid and 15 μg pyridoxamine.

### Mitsuokella Multacida Growth Conditions

Mitsuokella multacida (MM) was maintained frozen as glycerol stocks at −20° C. L-10 medium either liquid or solid (1% w/v agar) containing both maltose and glucose (0.1% w/v) or modified Scott and Dehority medium containing 10% (v/v) rumen fluid, 0.2% (w/v) glucose, 0.2% (w/v) cellobiose and 0.3% (v/v) starch [1] was used for culturing. Cultures were grown under anaerobic and stationary conditions in an atmosphere consisting of CO₂/H₂ (90:10) at a temperature of 37° C. or at 39° C. in 100% CO₂ in hungate tubes containing 5 ml of media.

### Mouse Model

Eight-week old male C57BL/6 mice (average initial weight 20 g) were purchased from the Chinese University of Hong Kong and allowed to acclimatize to the animal facility environment for two weeks prior to the experiments. In all experiments, male mice were used. All mice were kept at a strict 24 hr light-dark cycle, with lights on from 6 am to 6 pm. Every experimental group consisted of two cages per group to control for cage effect (n=5 per cage).

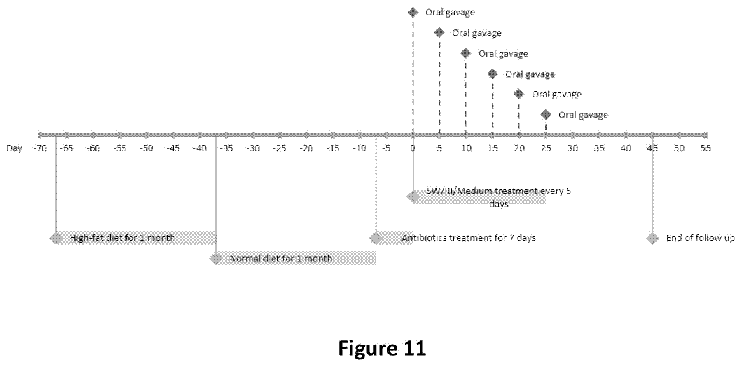

Mice were divided into 5 groups, SW, MM, RI, Medium and Consortium (a combination of SW, MM, and RI) group. Mice were administrated with three single bacteria at the dose of 1*10⁹ or Consortium (combined with 3 bacteria and each occupied for 33.33 percent) each 5 days. Body weight were measured every 5 days. Study design shown in FIG. 11.

### Antibiotics

For antibiotic treatment, mice were given a combination of kanamycin (0.4 mg/mL), gentamicin (0.035 mg/mL), colistin (850 U/mL), metronidazole (0.215 mg/mL), and vancomycin (0.045 mg/mL) in their drinking water for two weeks as previously described. Samples were immediately transferred to the animal facility in Hungate anaerobic culture tubes and the supernatant and bacteria was administered to the mice by oral gavage. Stool was collected on pre-determined days at the beginning of the dark phase, and immediately snap-frozen and transferred for storage at 80° C. until further processing.

### Lipid Profile

20 days post 6^{th} administration, mice were sacrificed by cervical dislocation for the collection of blood. Blood samples were collected in non-heparinized tubes and centrifuged at 1,600×g for 10 min at 4° C. Kits purchased from the Nanjing Jiancheng Bioengineering Institute (Nanjing, China) were used to determine the levels of serum TG (GPO-PAP assay; catalog no. A110-1), TC (GPO-PAP assay; catalog no. A111-1), LDL-C(catalog no. A113-1) and HDL-C(catalog no. A112-1), by Fisher Multiskan FC Multi-Detection Microplate Reader. The levels of TG and TC were determined at 510 nm and expressed as mmol/L. The levels of LDL-C and HDL-C were determined at 546 nm and expressed as mmol/L.

### Findings

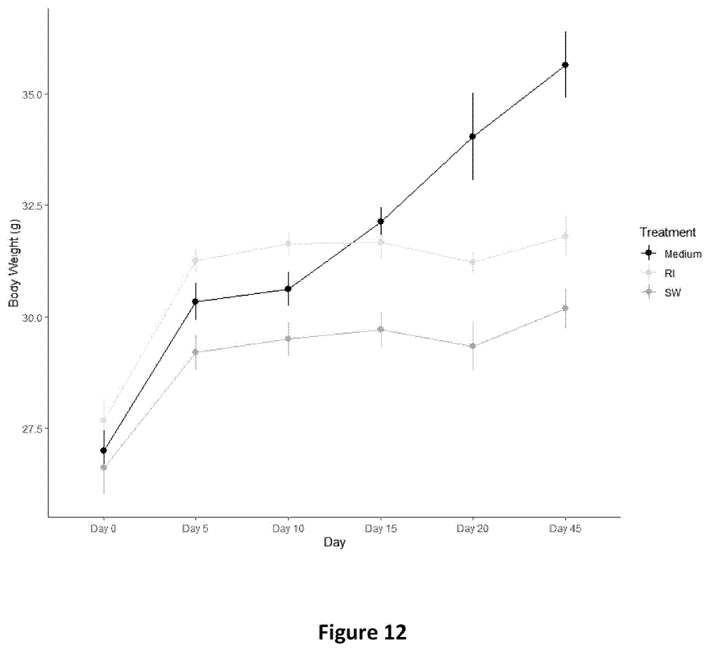

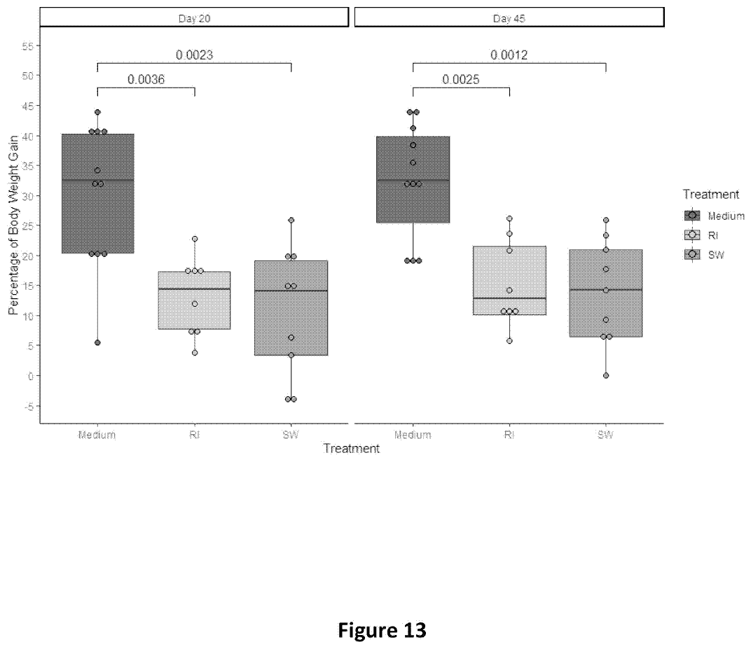

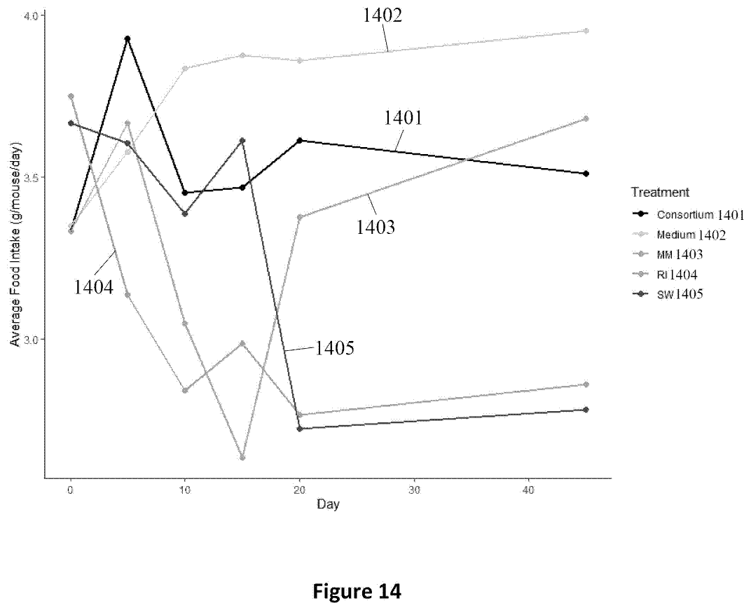

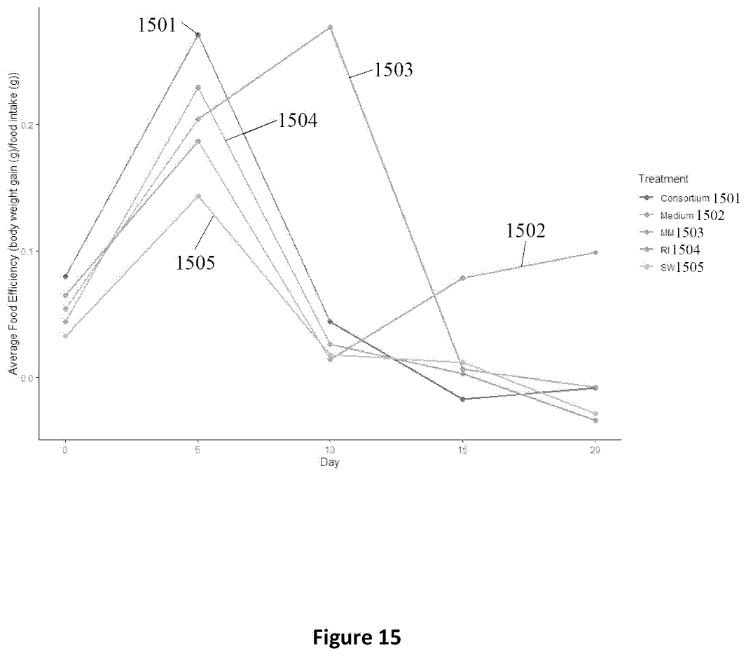

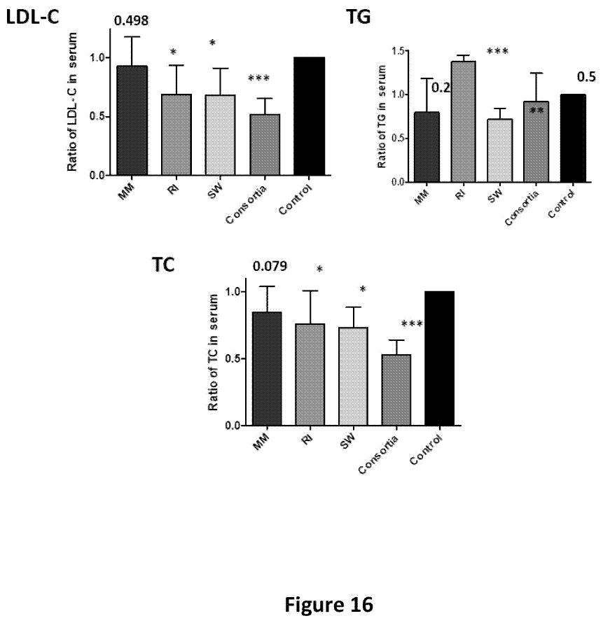

Administration of Sutterella wadsworthensis (SW) significantly reduced body weight gain (p<0.001; two-way repeated measures ANOVA) while and administration of Roseburia intestinalis (RI) showed a trend of reduced body weight gain. Effect is particular obviously after day 15 (after 3 oral gavage) (FIG. 12). Percentage of body weight gain of each mice on day 20 and day 45 compared to day 0 were calculated. Percentage of body weight gain was significantly lower on day 20 and day 45 in mice administered SW and RI in comparison to those administered medium (FIG. 13). The average food intake as gram of food intake per mouse per day was calculated. Average food intake showed a decreasing trend after administration of consortium (a combination of SW, MM, and RI), Mitsuokella multacida (MM), RI and SW, in contrast to administration of medium only which showed an increasing trend. The effect of reduction of food intake is most obvious in RI and SW (FIG. 14). Average food efficiency was calculated as the body weight gain (gram) in 5 days per total food intake (gram) in these 5 days per mouse. Average food efficiency were reduced after day 15 in mice administered with consortium, MM, RI and SW compared to administration of medium (FIG. 15). Effect on lipid metabolism was investigated. Administration of SW alone reduced LDL-C, total cholesterol (TC) and triglyceride (TG). Administration of RI alone reduced LDL-C and TC. Administration of consortium reduced LDL-C and TC. All results are compared to administration of medium-control on day 45 (FIG. 16).

ABBREVIATIONS sw: Sutterella wadsworthensis; ri-Roseburia intestinalis; mm: Mitsuokella multacida; HDL-C: high density lipoprotein cholesterol; LDL-C: low density lipoprotein cholesterol; TG: triglyceride; TC: total cholesterol; OGTT: Oral glucose tolerance test; ITT: Insulin tolerance test.

### EXAMPLE 8 CARBAPENEM-RESISTANT ENTEROBACTERIACEAE METHODS

### Clinical Trial of FMT for Eradication of CRE in Human

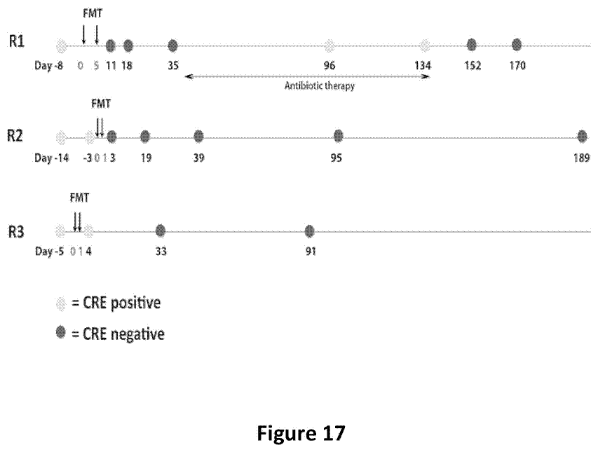

Patients who were age ≥18 years old, had two or more stool or rectal swab positive for CRE at least one week apart, and did not receive antimicrobial therapy for at least 48 hours prior to infusion of FMT were recruited to a clinical trial (NCT03479710). Patients who had active infection of CRE or VRE requiring antimicrobial therapy, pregnancy, active gastrointestinal tract infection or inflammatory disorders, had recent intro-abdominal surgery, had short gut syndrome or use of medications which alter gastrointestinal motility were excluded. CRE was defined as presence of any Enterobacteriaceae with resistance to any of the carbapenems. A total of 3 subjects with CRE, donors, and 4 healthy household subjects were recruited, and stool samples at baseline were obtained for analyses of microbiome. Timeline of sample collection for donor and recipients shown in FIG. 17.

In this study, patients received 2 FMT using frozen donor stool samples. 100 ml of FMT solution (raw stool 50 g) in 0.9% sterile saline were infused over 2-3 minutes into the distal duodenum or jejunum via oesophago-gastro-duodenoscopy (OGD). Stool samples were collected from patients before and after FMT prospectively. Recipients received FMT from the same single donor for the 2 FMTs.

Stool for FMT infusion were obtained from donors recruited to Stool Biobank for the Faculty of Medicine, The Chinese University of Hong Kong. Donors were volunteers from general population including spouses or partners, first-degree relatives, other relatives, friends and others who were known or unknown to the potential patients. Donors need to fulfil a set of eligibility criteria and passed screening laboratory tests for infectious diseases, including CRE and vancomycin-resistant Enterococcus (VRE).

### Fecal DNA Extraction

Approximately 100 mg fecal sample was prewashed with 1 ml ddH₂O and pelleted by centrifugation at 13,000×g for 1 min. The fecal pellet was resuspended in 800 μL TE buffer (pH 7.5), supplemented with 1.6 μl 2-mercaptoethanol and 500 U lyticase (Sigma), and incubated at 37° C. for 60 min. The sample was then centrifuged at 13,000×g for 2 min and the supernatant was discarded. After pretreatment, fecal DNA was subsequently extracted from the pellet using Maxwell® RSC PureFood GMO and Authentication Kit (Promega) following manufacturer's instructions. Briefly, fecal pellet was added 1 ml of CTAB buffer and vortexed for 30 seconds, then heating sample at 95° C. for 5 minutes. After that, the samples were vortexed thoroughly with beads at maximum speed for 15 min. Then 40 μl of proteinase K and 20 μl of RNase A was added into sample and the mixture was incubated at 70° C. for 10 minutes. The supernatant was then obtained by centrifuging at 13,000×g for 5 min and was added in Maxwell® RSC machine for DNA extraction. The extracted fecal DNA was subject to 16S rDNA sequencing and metagenomics sequencing.

### Metagenomics Sequencing and Analysis

Qualified fecal DNA was cut into fragments, the sequencing libraries were prepared through the processes of end repairing, adding A to tails, purification, and PCR amplification. The fecal DNA libraries were sequenced in-depth on the platform of Illumina Hiseqxten PE150 by the Beijing Genomics Institute (BGI) and yielded average 48±5.3 million reads (12G data) per sample.

The raw sequence reads were filtered and quality-trimmed by Trimmomatic v0.36 18 as follows: 1) trimming with a quality sliding window of 4:8; 2) cropping sequences to remove 20 bases from the start and bases beyond 220 from the end; 3) Removing sequences less than 150 bp long. Then the human host contaminate reads were filtering out by Kneaddata (https://bitbucket.org/biobakery/kneaddata/wiki/Home, Reference database: GRCh38 p12) with default argument to generate clean reads.

The bacteria taxonomic and functional profiling were implemented in humann2 v0.11.1 19, the process of which includes taxonomic identification by MetaPhlAn2 using clade-specific markers biomarker20, annotation of species pangenomes through Bowtie2 21 with ChocoPhlAn database, translated search of unmapped reads with DIAMOND 22 against UniRef90 universal protein reference database23, pathway collection was obtained from the generated gene list using Metacyc database 24. The gene families and pathway abundance files of all samples were joined and then normalized to relative abundance.

Taxonomic profile of fungi were determined from the fecal DNA metagenomic dataset using Kraken2 v2.0.7-beta. The full NCBI fungal and viral RefSeq database 25 was built from NCBI using Jellyfish program by counting distinct 31-mer in the reference libraries, with each k-mer in a read mapped to the lowest common ancestor of all reference genomes with exact k-mer matches. Each query was thereafter classified to a taxon with the highest total hits of k-mer matched by pruning the general taxonomic trees affiliated with mapped genomes.

### Statistics

The abundance data of bacteria, viruses, fungi were imported into R 3.3.5. Richness, diversity and rarefaction calculation were performed using the phyloseq package. The principal component analysis principal coordinates analysis (PCoA) based on the Bray-Curtis dissimilarity matrix of the microbial community structure was calculated by vegan R package. The heat maps were implemented using the ggplot R package.

### Findings

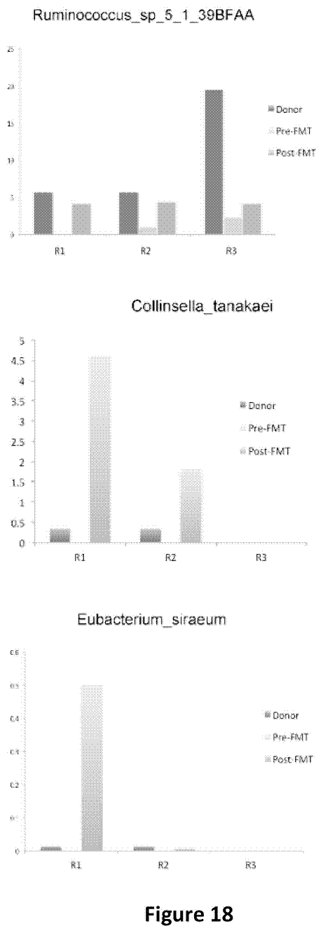

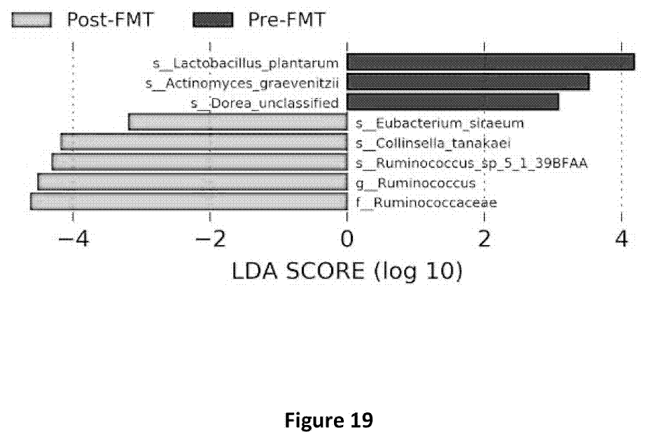

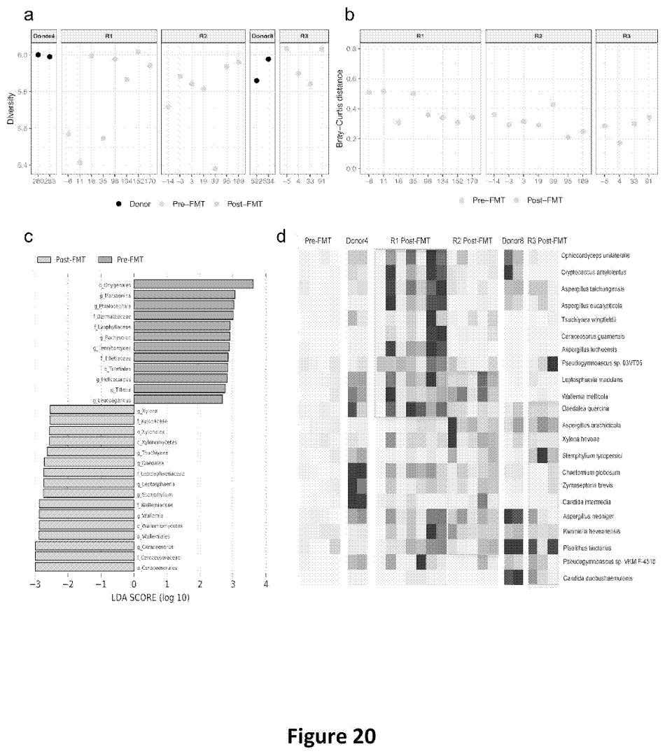

Relative abundance of Ruminococcus sp_5_1_39BFAA, Collinsella tanakaei and Eubacterium sicaeum were undetectable before FMT and increased after FMT to a level similar to donor or even higher (FIG. 18). LEfSe analysis also showed that these bacterial species were significantly increased in post-FMT samples compred to pre-FMT samples with LDA scores >2.0 (FIG. 20). These represents favorable bacteria for CRE decolonization (Table 15).

To address whether FMT impacts the gut mycobiome, fungi-enriched deep metagenomic sequencing was performed and then the sequence reads were aligned to reference databases to identify fungal taxonomy using Kraken2. Alpha diversity of the patient's gut fungal community showed extensive fluctuation over time after FMT (FIG. 20a) Fungal communities in post-FMT samples showed decreased dissimilarity to fungal communities from their corresponding donors (FIG. 20b), indicating possible engraftment of donor fungi in recipients or recovery of the recipients' fungal community towards a healthy state. Further discriminative analysis via LEfSe on the fecal mycobiome before and after FMT showed Xylonales, Wallemiales, Ceraceosorales as differentially enriched genera in post-FMT stool (FIG. 20c, LDA effect size >2, p<0.05). Further detailed species-level profiling revealed taxonomic differences of each recipient after FMT, for instance CRE patients engrafted 22 fungal species, including Daedalea quercina, Pisolithus tinctorius, and Aspergillus arachidicola (FIG. 20d, Table 16). While the transfer of microbiota after FMT primarily consisted of bacteria, some fungi engraftment may also be present, albeit in small numbers.

All patents, patent applications, and other publications, including GenBank Accession Numbers and other sequence identification numbers, cited in this application are incorporated by reference in the entirety for all purposes.

TABLE 1a
“Favourable” bacteria present in donors for FMT use to induce weight loss

| | | | | Minimum |
|---|---|---|---|---|
| | | | | relative |
| | | | | abundance* |
| | | | | in donor for |
| No. | Study | Analysis | Taxa* (family, genus, species, strains) | FMT (%) |
| 1 | Human to Mice FMT | LEfSe | Bifidobacterium_{—}bifidum (species) | 1.7308 |
| 2 | Human to Mice FMT | LEfSe | Acidaminococcaceae (family) | 1.6421 |
| 3 | Human to Mice FMT | LEfSe | Acidaminococcus (genus) | 1.2371 |
| 4 | Human to Mice FMT | LEfSe, Correlation (Linear | Roseburia intestinalis (species) | 1.1788 |
| | | Regression) | | |
| 5 | Human to Mice FMT | LEfSe | Bacteroides_{—}intestinalis (species) | 0.1893 |
| 6 | Human to Mice FMT | LEfSe | Peptostreptococcaceae (family) | 0.141 |
| 7 | Human to Mice FMT | LEfSe | Parabacteroides_{—}goldsteinii (species) | 0.0638 |
| 8 | Human to Mice FMT | LEfSe | Veillonella_{—}atypica (species) | 0.0406 |
| 9 | Human to Mice FMT | LEfSe | Alistipes indistinctus (species) | 0.0231 |
| 10 | Human to Mice FMT | LEfSe | Streptococcus_{—}infantis (species) | 0.0101 |
| 11 | Human to Mice FMT | LEfSe | Actinomycetaceae (family) | 0.0045 |
| 12 | Human to Mice FMT | LEfSe | Actinomyces (genus) | 0.0045 |
| 13 | Human to Mice FMT | LEfSe | Citrobacter_unclassified (species) | 0.0039 |
| 14 | Human to Mice FMT | Correlation (Linear Regression) | Sutterella wadsworthensis (species) | |
| 15 | Human to Mice FMT | Correlation (Linear Regression) | Olsenella unclassified (genus) | |
| 16 | Human to Mice FMT | Correlation (Linear Regression) | Mitsuokella multacida (species) | |
| 17 | Human to Mice FMT | Correlation (Linear Regression) | Citrobacte unclassified (genus) | |

*Relative abundance calculated as number of all sequence belonging to the taxa divided by number of all sequence detected in the sample

TABLE 1b
Maximum “unfavourable” bacteria in donors for the use of FMT to induce weight loss

| | | | | Maximum |
|---|---|---|---|---|
| | | | | abundance |
| | | | | in donor for |
| No. | Study | Analysis | Taxa* (family, genus, species, strains) | FMT (%) |
| 1 | Human to Mice FMT | LEfSe | Oscillibacter (genus) | 0.3737 |
| 2 | Human to Mice FMT | LEfSe | Ruminococcus_{—}gnavus (species) | 0.354 |
| 3 | Human to Mice FMT | LEfSe | Bifidobacterium_{—}catenulatum (species) | 0.0337 |
| 4 | Human to Mice FMT | LEfSe | Coriobacteriaceae_{—}bacterium_phl (species) | 0.0279 |
| 5 | Human to Mice FMT | LEfSe | Adlercreutzia_{—}equolifaciens (species) | 0.024 |
| 6 | Human to Mice FMT | LEfSe | Blautia_{—}hydrogenotrophica (species) | 0.0055 |
| 7 | Human to Mice FMT | LEfSe | Erysipelotrichaceae_{—}bacterium_2_2_44A | 0.001 |
| | | | (species) | |
| 8 | Human to Mice FMT | Correlation (Linear Regression) | Bacteroides sp_3_1_19 (species) | |
| 9 | Human to Mice FMT | Correlation (Linear Regression) | Burkholderiales bacterium_1_1_47 | |
| | | | (species) | |
| 10 | Human to Mice FMT | Correlation (Linear Regression) | Citrobacter koseri (species) | |
| 11 | Human to Mice FMT | Correlation (Linear Regression) | Dorea formicigenerans (species) | |
| 12 | Human to Mice FMT | Correlation (Linear Regression) | Eubacterium hallii (species) | |
| 13 | Human to Mice FMT | Correlation (Linear Regression) | Eubacterium rectale (species) | |
| 14 | Human to Mice FMT | Correlation (Linear Regression) | Faecalibacterium prausnitzii (species) | |
| 15 | Human to Mice FMT | Correlation (Linear Regression) | Haemophilus pittmaniae (species) | |
| 16 | Human to Mice FMT | Correlation (Linear Regression) | Haemophilus sputorum (species) | |
| 17 | Human to Mice FMT | Correlation (Linear Regression) | Klebsiella oxytoca (species) | |
| 18 | Human to Mice FMT | Correlation (Linear Regression) | Klebsiella unclassified (genus) | |
| 19 | Human to Mice FMT | Correlation (Linear Regression) | Lachnospiraceae bacterium_5_1_63FAA | |
| | | | (species) | |
| 20 | Human to Mice FMT | Correlation (Linear Regression) | Parasutterella excrementihominis (species) | |
| 21 | Human to Mice FMT | Correlation (Linear Regression) | Peptostreptococcaceae_noname | |
| | | | unclassified (species) | |
| 22 | Human to Mice FMT | Correlation (Linear Regression) | Porphyromonas endodontalis (species) | |
| 23 | Human to Mice FMT | Correlation (Linear Regression) | Prevotella disiens (species) | |
| 24 | Human to Mice FMT | Correlation (Linear Regression) | Ruminococcus obeum (species) | |

*Relative abundance calculated as number of all sequence belonging to the taxa divided by number of all sequence detected in the sample

TABLE 2
“Favourable” fungi in donors for the use of FMT to induce weight loss

| | | | Minimum relative |
|---|---|---|---|
| | | | abundance* in donor for |
| Study | Analysis | Taxa (species) | FMT (%) |
| Cross-sectional Human | LEfSe | Cercospora beticola | 5.6 |
| Study | | | |
| Cross-sectional Human | LEfSe | Kazachstania naganishii | 0.6 |
| Study | | | |

*Relative abundance calculated as number of all sequence belonging to the taxa divided by number of all sequence detected in the sample

TABLE 3a
Fungal species in recipient associated with successful weight loss

| | | | Minimum relative |
|---|---|---|---|
| | | | abundance* in recipient to |
| | | | predict successful weight |
| Study | Analysis | Taxa (species) | loss (%) |
| Randomized controlled | LEfSe | Komagataella phaffii | 0.54 |
| trial | | | |

*Relative abundance calculated as number of all sequence belonging to the taxa divided by number of all sequence detected in the sample

TABLE 3b
Fungal species in recipient associated with failure of weight loss

| | | | Minimum relative |
|---|---|---|---|
| | | | abundance* in recipient |
| | | | that predict unsuccessful |
| Study | Analysis | Taxa (species) | weight loss (%)^{#} |
| Randomized controlled | LEfSe | Kluyveromyces lactis | 1.13 |
| trial | | | |
| Randomized controlled | LEfSe | Torulaspora delbrueckii | 0.85 |
| trial | | | |
| Randomized controlled | LEfSe | Encephalitozoon | 0.073 |
| trial | | romaleae | |

*Relative abundance calculated as number of all sequence belonging to the taxa divided by number of all sequence detected in the sample
^{#}To enhance efficacy of weight loss intervention, relative abundance in recipient should be kept below these value

TABLE 4
“Favourable” bacteria in donors for the use of FMT to treat CRE

| | | | | Minimum | Average |
|---|---|---|---|---|---|
| | | | | relative | fold |
| | | | | abundance* | change in |
| | | | | in donor for | recipient |
| No. | Study | Analysis | Taxa (species) | FMT (%) | after FMT |
| 1 | Prospective pilot | Fold change | Lachnospiraceae_{—}bacterium_{—} | 1.105 | 838.222 |
| | | | 5_1_63FAA | | |
| 2 | Prospective pilot | Fold change | Escherichia_{—}coli | 2.752 | 119.3285 |
| 3 | Prospective pilot, | Fold change, | Faecalibacterium_{—}prausnitzii | 2.45 | 103.186 |
| | Cross-sectional | LEfSe | | | |
| 4 | Prospective pilot, | Fold change, | Ruminococcus_{—}obeum | 3.797 | 72.608 |
| | Cross-sectional | LEfSe | | | |
| 5 | Prospective pilot | Fold change | Clostridium_{—}bolteae | 1.134 | 26.013 |
| 6 | Prospective pilot | Fold change | Dorea_{—}formicigenerans | 1.376 | 19.018 |
| 7 | Prospective pilot | Fold change | Parasutterella_{—}excrem4entihominis | 0.104 | 17.315 |
| 8 | Prospective pilot, | Fold change, | Burkholderiales_{—}bacterium_{—} | 0.226 | 16.325 |
| | Cross-sectional | LEfSe | 1_1_47 | | |
| 9 | Prospective pilot | Fold change | Clostridium_{—}scindens | 0.002 | 14.625 |
| 10 | Prospective pilot | Fold change | Clostridium_{—}citroniae | 0.015 | 13.429 |
| 11 | Prospective pilot | Fold change | Erysipelotrichaceae_{—}bacterium_{—} | 0.025 | 10.21 |
| | | | 21_3 | | |
| 12 | Prospective pilot | Fold change | Eubacterium_{—}limosum | 0.709 | 7.224 |
| 13 | Prospective pilot | Fold change | Gordonibacter_{—}pamelaeae | 0.014 | 6.223 |
| 14 | Prospective pilot | Fold change | Streptococcus_{—}mitis_{—}oralis_{—} | 0.2 | 5.814 |
| | | | pneumoniae | | |
| 15 | Prospective pilot | Fold change | Lachnospiraceae_{—}bacterium_{—} | 0.042 | 5.319 |
| | | | 1_4_56FAA | | |
| 16 | Prospective pilot, | Fold change, | Anaerostipes_{—}hadrus | 0.69 | 3.611 |
| | Cross-sectional | LEfSe | | | |
| 17 | Prospective pilot | Fold change | Streptococcus_{—}australis | 0.023 | 3.437 |
| 18 | Prospective pilot | Fold change | Alistipes_{—}shahii | 0.051 | 3.31 |
| 19 | Prospective pilot, | Fold change, | Lachnospiraceae_{—}bacterium_{—} | 0.394 | 3.239 |
| | Cross-sectional | LEfSe | 1_1_57FAA | | |
| 20 | Prospective pilot | Fold change | Streptococcus_{—}infantis | 0.031 | 3.001 |
| 21 | Prospective pilot | Fold change | Bifidobacterium_{—}longum | 6.738 | 2.6875 |
| 22 | Prospective pilot | Fold change | Rothia_{—}mucilaginosa | 0.011 | 2.655 |
| 23 | Prospective pilot, | Fold change, | Alistipes_{—}putredinis | 6.452 | 2.457 |
| | Cross-sectional | LEfSe | | | |
| 24 | Prospective pilot, | Fold change, | Ruminococcus_sp_5_1_39BFAA | 5.59 | 2.43 |
| | Cross-sectional | LEfSe | | | |
| 25 | Prospective pilot | Fold change | Roseburia_{—}inulinivorans | 0.173 | 2.374 |
| 26 | Prospective pilot | Fold change | Erysipelotrichaceae_{—}bacterium_{—} | 0.042 | 2.045 |
| | | | 6_1_45 | | |
| 27 | Prospective pilot | Fold change | Streptococcus_{—}salivarius | 0.722 | 1.353 |
| 28 | Prospective pilot | Fold change | Collinsella_{—}aerofaciens | 2.619 | 1.282 |
| 29 | Prospective pilot | Fold change | Erysipelotrichaceae_{—}bacterium_{—} | 0.168 | 1.16 |
| | | | 2_2_44A | | |
| 30 | Prospective pilot | Fold change | Clostridium_{—}leptum | 0.106 | 1.123 |
| 31 | Cross-sectional | LEfSe | Actinomyces_{—}massiliensis. | | |
| 32 | Cross-sectional | LEfSe | Alistipes_{—}finegoldii | | |
| 33 | Cross-sectional | LEfSe | Alistipes_{—}onderdonkii | | |
| 34 | Cross-sectional | LEfSe | Bacteroides_{—}dorei | | |
| 35 | Cross-sectional | LEfSe | Bacteroides_{—}eggerthii | | |
| 36 | Cross-sectional | LEfSe | Bacteroides_{—}nordii | | |
| 37 | Cross-sectional | LEfSe | Bacteroides_{—}ovatus | | |
| 38 | Cross-sectional | LEfSe | Bacteroides_{—}thetaiotaomicron | | |
| 39 | Cross-sectional | LEfSe | Bacteroides_{—}uniformis | | |
| 40 | Cross-sectional | LEfSe | Dorea_{—}longicatena | | |
| 41 | Cross-sectional | LEfSe | Enterobacter_{—}aerogenes | | |
| 42 | Cross-sectional | LEfSe | Enterococcus_{—}avium | | |
| 43 | Cross-sectional | LEfSe | Enterococcus_{—}faecalis | | |
| 44 | Cross-sectional | LEfSe | Eubacterium_{—}dolichum | | |
| 45 | Cross-sectional | LEfSe | Eubacterium_{—}hallii | | |
| 46 | Cross-sectional | LEfSe | Eubacterium_{—}ramulus | | |
| 47 | Cross-sectional | LEfSe | Eubacterium_{—}rectale | | |
| 48 | Cross-sectional | LEfSe | Lachnospiraceae_{—}bacterium_{—} | | |
| | | | 2_1_58FAA | | |
| 49 | Cross-sectional | LEfSe | Parabacteroides_{—}goldsteinii | | |
| 50 | Cross-sectional | LEfSe | Parabacteroides_{—}merdae | | |
| 51 | Cross-sectional | LEfSe | Streptococcus_{—}intermedius | | |
| 52 | Cross-sectional | LEfSe | Streptococcus_{—}macedonicus | | |
| 53 | Cross-sectional | LEfSe | Streptococcus_{—}thermophilus | | |

*Relative abundance calculated as number of all sequence belong to the taxa divided by number of all sequence detected in the sample

TABLE 5
“Favourable” fungi in donors for the use of FMT to treat CRE

| | | | | | Average |
|---|---|---|---|---|---|
| | | | | | fold |
| | | | | Minimum relative | change in |
| | | | | abundance* in | recipient |
| No. | Study | Analysis | Taxa (species) | donor for FMT (%) | after FMT |
| 1 | Prospective pilot trial | Fold change | Encephalitozoon hellem | 0.025 | 45.175 |
| 2 | Prospective pilot trial | Fold change | Naumovozyma castellii | 0.011 | 4.912 |
| 3 | Prospective pilot trial | Fold change | Candida orthopsilosis | 0.012 | 3.17 |
| 4 | Prospective pilot trial | Fold change | Cercospora beticola | 0.071 | 2.045 |
| 5 | Prospective pilot trial | Fold change | Schizosaccharomyces pombe | 0.156 | 1.973 |
| 6 | Prospective pilot trial | Fold change | Yarrowia lipolytica | 0.016 | 1.83 |
| 7 | Prospective pilot trial | Fold change | Cryptococcus gattii VGI | 0.011 | 1.793 |
| 8 | Prospective pilot trial | Fold change | Naumovozyma dairenensis | 0.012 | 1.72 |
| 9 | Prospective pilot trial | Fold change | Kluyveromyces marxianus | 0.016 | 1.574 |
| 10 | Prospective pilot trial | Fold change | Debaryomyces hansenii | 0.012 | 1.541 |
| 11 | Prospective pilot trial | Fold change | Scheffersomyces stipitis | 0.01 | 1.532 |
| 12 | Prospective pilot trial | Fold change | Torulaspora delbrueckii | 0.012 | 1.512 |
| 13 | Prospective pilot trial | Fold change | Fusarium fujikuroi | 0.013 | 1.471 |
| 14 | Prospective pilot trial | Fold change | Eremothecium gossypii | 0.003 | 1.404 |
| 15 | Prospective pilot trial | Fold change | Thielavia terrestris | 0.053 | 1.328 |
| 16 | Prospective pilot trial | Fold change | Zymoseptoria tritici | 0.013 | 1.3245 |
| 17 | Prospective pilot trial | Fold change | Thermothelomyces thermophila | 0.038 | 1.317 |
| 18 | Prospective pilot trial | Fold change | Fusarium oxysporum | 0.014 | 1.265 |
| 19 | Prospective pilot trial | Fold change | Ogataea parapolymorpha | 0.01 | 1.2635 |
| 20 | Prospective pilot trial | Fold change | Candida glabrata | 0.008 | 1.247 |
| 21 | Prospective pilot trial | Fold change | Neurospora crassa | 0.033 | 1.246 |
| 22 | Prospective pilot trial | Fold change | Aspergillus fumigatus | 0.023 | 1.237 |
| 23 | Prospective pilot trial | Fold change | Fusarium venenatum | 0.015 | 1.234 |
| 24 | Prospective pilot trial | Fold change | Ustilago maydis | 0.02 | 1.194 |
| 25 | Prospective pilot trial | Fold change | Fusarium graminearum | 0.007 | 1.142 |
| 26 | Prospective pilot trial | Fold change | Lachancea thermotolerans | 0.01 | 1.134 |
| 27 | Prospective pilot trial | Fold change | Eremothecium sinecaudum | 0.03 | 1.061 |
| 28 | Prospective pilot trial | Fold change | Magnaporthe oryzae | 0.025 | 1.046 |

*Relative abundance calculated as number of all sequence belong to the taxa divided by number of all sequence detected in the sample

TABLE 6a
“Favourable” bacteria in donors for the use of FMT to treat acute Graft versus host disease

| | | | | Minimum relative |
|---|---|---|---|---|
| | | | | abundance* in donor |
| No. | Study | Analysis | Taxa (species) | for FMT (%) |
| 1 | Case Report | Heatmap | Ruminococcus gnavus | 4.4 |
| 2 | Case Report | Heatmap | Eggerthella unclassified | 1.3 |
| 3 | Case Report | Heatmap, Correlation with | Clostridium nexile | 1.1 |
| | | symptom | | |
| 4 | Case Report | Heatmap, Correlation with | Alistipes putredinis | 1 |
| | | symptom, LEFSe (Symptoms) | | |
| 5 | Case Report | Heatmap, LEFSe (Symptoms) | Alistipes onderdonkii | 0.1 |
| 6 | Case Report | Heatmap, Correlation with | Clostridium bolteae | 0.1 |
| | | symptom | | |
| 7 | Case Report | Heatmap | Clostridium symbiosum | 0.1 |
| 8 | Case Report | LEFSe (Symptoms), | Clostridium hathewayi | |
| | | Correlation with symptom | | |

TABLE 6b
Maximum abundance of “unfavourable” bacterial
species in donors for using FMT to treat aGvHD

| | | | | Maximum relative |
|---|---|---|---|---|
| | | | | abundance* in donor |
| No. | Study | Analysis | Taxa (species) | for FMT (%) |
| 1 | Case Report | Heatmap, Correlation with | Corynebacterium jeikeium | 0 |
| | | symptom | | |
| 2 | Case Report | LEFSe (Symptoms) | Eubacterium rectale | |

TABLE 7a
“Favourable” fungal species in donors for using FMT to treat aGvHD

| | | | | Minimum |
|---|---|---|---|---|
| | | | | relative |
| | | | | abundance* |
| | | | | in donor for |
| No. | Study | Analysis | Taxa | FMT (%) |
| 1 | Case Report | Heatmap | Schizosaccharomyces pombe | 15.6 |
| 2 | Case Report | Heatmap | Cercospora beticola | 7.1 |
| 3 | Case Report | Heatmap, Correlation with symptom | Colletotrichum higginsianum | 3.9 |
| 4 | Case Report | Heatmap, Correlation with symptom | Neurospora crassa | 3 |
| 5 | Case Report | Heatmap, Correlation with symptom | Thermothelomyces | 2.9 |
| | | | thermophila | |
| 6 | Case Report | Heatmap, Correlation with symptom | Fusarium pseudograminearum | 2.4 |
| 7 | Case Report | Heatmap, Correlation with symptom | Thielavia terrestris | 2 |
| 8 | Case Report | Heatmap | Eremothecium sinecaudum | 1.7 |
| 9 | Case Report | Heatmap | Fusarium verticillioides | 1.7 |
| 10 | Case Report | Heatmap, Correlation with symptom | Fusarium venenatum | 1.5 |
| 11 | Case Report | Heatmap | Candida dubliniensis | 1.4 |
| 12 | Case Report | Heatmap, Correlation with symptom | Eremothecium gossypii | 1.4 |
| 13 | Case Report | Correlation with symptom | [Candida] glabrata | |
| 14 | Case Report | Correlation with symptom | Aspergillus fumigatus | |
| 15 | Case Report | Correlation with symptom | Encephalitozoon hellem | |
| 16 | Case Report | Correlation with symptom | Encephalitozoon intestinalis | |
| 17 | Case Report | Correlation with symptom | Eremothecium cymbalariae | |
| 18 | Case Report | Correlation with symptom | Lachancea thermotolerans | |
| 19 | Case Report | Correlation with symptom | Ogataea parapolymorpha | |
| 20 | Case Report | Correlation with symptom | Pochonia chlamydosporia | |
| 21 | Case Report | Correlation with symptom | Saccharomyces eubayanus | |
| 22 | Case Report | Correlation with symptom | Sugiyamaella lignohabitans | |
| 23 | Case Report | Correlation with symptom | Torulaspora delbrueckii | |
| 24 | Case Report | Correlation with symptom | Zymoseptoria tritici | |

TABLE 7b
“Unfavourable” fungal species in donors for using FMT to treat aGvHD

| | | | | Maximum |
|---|---|---|---|---|
| | | | | relative |
| | | | | abundance* |
| | | | | in donor for |
| No | Study | Analysis | Taxa | FMT (%) |
| 1 | Case Report | Heatmap, Correlation with symptom | Botrytis cinerea | 3.2 |
| 2 | Case Report | Heatmap, Correlation with symptom | Fusarium oxysporum | 1.4 |

TABLE 8a
Viral taxa over-represented in ulcerative colitis

| | | Fold change compared to healthy |
|---|---|---|
| No. | Taxa (family/genus/species) | population (log₂ transformed) |
| 1 | Sodalis_phage (species) | 4.9077 |
| 2 | Phix174microvirus (genus) | 3.8329 |
| 3 | Escherichia_phage (species) | 3.3513 |
| 4 | Mimivirus (species) | 3.0426 |
| 5 | Enterobacteria_phage (species) | 2.6909 |
| 6 | P1virus (genus) | 2.6352 |
| 7 | Propionibacterium_phage (species) | 2.3084 |
| 8 | Cellulophaga_phage (species) | 2.0411 |
| 9 | Microviridae (family) | 1.6509 |
| 10 | Rhizobium_phage (species) | 1.4602 |
| 11 | Orthopneumovirus (genus) | 1.1122 |
| 12 | Human_respiratory_syncytial_virus (species) | 1.0094 |
| 13 | Acidithiobacillus_phage (species) | 1.008 |
| 14 | Lambdavirus (genus) | 0.9913 |
| 15 | Chrysochromulina_ericina_virus (species) | 0.9154 |
| 16 | P22virus (genus) | 0.9066 |
| 17 | Pneumoviridae (family) | 0.8928 |
| 18 | T4virus (genus) | 0.8646 |
| 19 | Myoviridae (family) | 0.7369 |
| 20 | Podoviridae (family) | 0.5781 |
| 21 | Planktothrix_phage (species) | 0.5488 |

TABLE 8b
Viral taxa under-represented in UC

| | | Fold change compared to healthy |
|---|---|---|
| No. | Taxa (family/genus/species) | population (log₂ transformed) |
| 1 | Anelloviridae (family) | −4.6378 |
| 2 | Phaeocystis_globosa_virus (species) | −1.5589 |
| 3 | Bathycoccus_sp_RCC1105_virus (species) | −1.3418 |
| 4 | uncultured_phage (species) | −1.2617 |
| 5 | Powai_lake_megavirus (species) | −1.102 |
| 6 | Mimivirus (genus) | −0.8692 |
| 7 | Orthopoxvirus (genus) | −0.8657 |
| 8 | Coccolithovirus (genus) | −0.7681 |
| 9 | uncultured_Mediterranean_phage (species) | −0.4914 |

TABLE 9a
Viral species over-represented in Enterotype
2 compared to Enterotype 1

| | Fold change compared to healthy |
|---|---|
| Taxa (species) | population (log₂ transformed) |
| Escherichia_phage | 5.40 |
| Cellulophaga_phage | 2.47 |
| uncultured_Caudovirales_phage | 1.17 |
| Enterobacteria_phage | 4.48 |
| Pseudomonas_phage | 1.04 |
| Tupanvirus | 1.34 |
| Acinetobacter_phage | 1.77 |
| Salmonella_phage | 1.22 |
| Staphylococcus_phage | 1.19 |
| Mycobacterium_phage | 2.17 |
| Acidithiobacillus_phage | 1.12 |
| Vibrio_phage | 2.19 |
| Aeromonas_phage | 2.38 |
| Caulobacter_phage | 1.76 |
| Rhizobium_phage | 2.89 |
| Tetraselmis_virus | 1.07 |
| Yellowstone_lake_phycodnavirus | 1.63 |
| Stx2-converting_phage | 1.60 |
| Propionibacterium_phage | 2.92 |
| Enterobacter_phage | 2.18 |
| Megavirus | 2.06 |
| Micromonas_sp_RCC1109_virus | 1.15 |
| Caulobacter_virus | 2.19 |
| Brazilian_cedratvirus | 1.27 |
| Sodalis_phage | 3.50 |

TABLE 9b
Viral species under-represented in Enterotype
2 compared to Enterotype 1

| | Fold change of |
|---|---|
| | Enterotype 2 vs |
| | Enterotype 1 |
| Taxa (species) | (log₂ transformed) |
| Indivirus | −1.33 |
| Ralstonia_phage | −1.61 |
| Thermus_phage | −1.45 |
| Powai_lake_megavirus | −2.66 |
| Bathycoccus_sp_RCC1105_virus | −2.82 |
| Bacillus_thuringiensis_phage | −1.33 |
| Klebsiella_phage | −2.16 |
| Aeropyrum_pernix_spindle-shaped_virus | −3.26 |
| Rhodococcus_phage | −2.02 |
| uncultured_marine_virus | −1.59 |
| Lysinibacillus_phage | −3.02 |
| Brevibacillus_phage | −1.42 |
| Ochrobactrum_phage | −2.96 |
| Serratia_phage | −1.51 |
| Orpheovirus | −1.36 |
| Feldmannia_irregularis_virus | −1.62 |
| Streptomyces_phage | −1.76 |
| Clostridium_phage | −1.50 |
| Ostreococcus_lucimarinus_virus | −2.53 |
| Flavobacterium_phage | −2.73 |
| Acanthamoeba_castellanii_mamavirus | −2.51 |
| Salinibacter_virus | −3.38 |
| Xanthomonas_phage | −1.40 |
| Bacteriophage | −3.85 |
| Phaeocystis_globosa_virus | −2.60 |
| Pike_perch_iridovirus | −3.53 |
| Dickeya_phage | −1.40 |
| Acidianus_two-tailed_virus | −2.17 |

TABLE 10a
Bacterial taxa that are over-represented in UC

| Taxa (family/genus/species) | LDA effect size |
|---|---|
| Firmicutes (phylum) | 5.5610 |
| Pseudomonadaceae (family) | 5.2509 |
| Ruminococcaceae (family) | 4.6693 |
| Thermaceae (family) | 3.6370 |
| Clostridiales_unclassified (family) | 3.5887 |
| Veillonellaceae (family) | 3.5867 |
| Coprococcus (genus) | 3.9023 |

TABLE 10b
Bacterial taxa that are under-represented in UC

| Taxa (family/genus/species) | LDA effect size |
|---|---|
| Proteobacteria (phylum) | 5.8313 |
| Actinobacteria (phylum) | 4.7581 |
| Fusobacteria (phylum) | 4.5352 |
| Oxalobacteraceae (family) | 5.6387 |
| Fusobacteriaceae (family) | 4.5406 |
| Cellulomonadaceae (family) | 4.4426 |
| Fusobacterium (genus) | 4.5392 |
| Massilia (genus) | 5.6450 |
| Escherichia (genus) | 4.7503 |
| Cellulomonas (genus) | 4.4122 |

TABLE 11
Supplementation of bacteria species that can enhance
FMT efficacy in Crohn's Disease patients with AIEC

| No* | Study | Analysis | Species |
|---|---|---|---|
| 1 | Mice to Mice FMT | LEfSe | Akkermansia_muciniphila_Otu00007 |
| 2 | Mice to Mice FMT | LEfSe | Allobaculum_Otu00012 |
| 3 | Mice to Mice FMT | LEfSe | S24_7_Otu00017 |
| 4 | Mice to Mice FMT | LEfSe | Allobaculum_Otu00018 |
| 5 | Mice to Mice FMT | LEfSe | Akkermansia_muciniphila_Otu00035 |
| 6 | Mice to Mice FMT | LEfSe | S24_7_Otu00059 |
| 7 | Mice to Mice FMT | LEfSe | Sutterella_Otu00051 |
| 8 | Mice to Mice FMT | LEfSe | S24_7_Otu00048 |
| 9 | Mice to Mice FMT | LEfSe | Allobaculum_Otu00050 |
| 10 | Mice to Mice FMT | LEfSe | S24_7_Otu00041 |
| 11 | Mice to Mice FMT | LEfSe | S24_7_Otu00110 |
| 12 | Mice to Mice FMT | LEfSe | Dorea_Otu00090 |
| 13 | Mice to Mice FMT | LEfSe | S24_7_Otu00040 |
| 14 | Mice to Mice FMT | LEfSe | Helicobacteraceae_Otu00016 |
| 15 | Mice to Mice FMT | LEfSe | Helicobacteraceae_Otu00020 |
| 16 | Mice to Mice FMT | LEfSe | Thiobacillus_Otu00037 |
| 17 | Mice to Mice FMT | LEfSe | Sinobacteraceae_Otu00073 |
| 18 | Mice to Mice FMT | LEfSe | Sutterella_Otu00253 |
| 19 | Mice to Mice FMT | LEfSe | Escherichia_coli_Otu00024 |
| 20 | Mice to Mice FMT | LEfSe | S24_7_Otu00252 |
| 21 | Mice to Mice FMT | LEfSe | Prevotella_Otu00286 |
| 22 | Mice to Mice FMT | LEfSe | Prevotella_copri_Otu00100 |
| 23 | Mice to Mice FMT | LEfSe | Allobaculum_Otu00715 |
| 24 | Mice to Mice FMT | LEfSe | Rhodobacteraceae_Otu00405 |
| 25 | Mice to Mice FMT | LEfSe | Lactobacillus_Otu00228 |
| 26 | Mice to Mice FMT | LEfSe | Bacteroidetes_Otu00349 |
| 27 | Mice to Mice FMT | LEfSe | Akkermansia_muciniphila_Otu00462 |
| 28 | Mice to Mice FMT | LEfSe | Ruminococcus_gnavus_Otu00848 |
| 29 | Mice to Mice FMT | LEfSe | Bacteroides_plebeius_Otu00282 |
| 30 | Mice to Mice FMT | LEfSe | S24_7_Otu00381 |
| 31 | Mice to Mice FMT | LEfSe | SHA_20_Otu00319 |
| 32 | Mice to Mice FMT | LEfSe | Chromatiaceae_Otu00422 |
| 33 | Mice to Mice FMT | LEfSe | Desulfarculaceae_Otu00318 |
| 34 | Mice to Mice FMT | LEfSe | S24_7_Otu00283 |
| 35 | Mice to Mice FMT | LEfSe | Capnocytophaga_Otu00270 |
| 36 | Mice to Mice FMT | LEfSe | S24_7_Otu00264 |
| 37 | Mice to Mice FMT | LEfSe | Christensenellaceae_Otu00288 |
| 38 | Mice to Mice FMT | LEfSe | Helicobacteraceae_Otu00146 |
| 39 | Mice to Mice FMT | LEfSe | OM60_Otu00403 |
| 40 | Mice to Mice FMT | LEfSe | Desulfococcus_Otu00390 |
| 41 | Mice to Mice FMT | LEfSe | Piscirickettsiaceae_Otu00393 |
| 42 | Mice to Mice FMT | LEfSe | S24_7_Otu00122 |
| 43 | Mice to Mice FMT | LEfSe | Akkermansia_muciniphila_Otu00499 |
| 44 | Mice to Mice FMT | LEfSe | S24_7_Otu00212 |
| 45 | Mice to Mice FMT | LEfSe | Allobaculum_Otu00542 |
| 46 | Mice to Mice FMT | LEfSe | S24_7_Otu00395 |
| 47 | Mice to Mice FMT | LEfSe | S24_7_Otu00461 |
| 48 | Mice to Mice FMT | LEfSe | Burkholderiales_Otu00278 |
| 49 | Mice to Mice FMT | LEfSe | Desulfococcus_Otu00320 |
| 50 | Mice to Mice FMT | LEfSe | Cardiobacterium_Otu00762 |
| 51 | Mice to Mice FMT | LEfSe | Sinobacteraceae_Otu00267 |
| 52 | Mice to Mice FMT | LEfSe | Bacillus_cereus_Otu00268 |
| 53 | Mice to Mice FMT | LEfSe | Betaproteobacteria_Otu00269 |
| 54 | Mice to Mice FMT | LEfSe | Faecalibacterium_prausnitzii_Otu00260 |
| 55 | Mice to Mice FMT | LEfSe | S24_7_Otu01040 |
| 56 | Mice to Mice FMT | LEfSe | Roseburia_faecis_Otu00457 |
| 57 | Mice to Mice FMT | LEfSe | Lachnospiraceae_Otu00522 |
| 58 | Mice to Mice FMT | LEfSe | S24_7_Otu00564 |
| 59 | Mice to Mice FMT | LEfSe | Piscirickettsiaceae_Otu00407 |
| 60 | Mice to Mice FMT | LEfSe | Clostridia_Otu00298 |
| 61 | Mice to Mice FMT | LEfSe | LCP_6_Otu00406 |
| 62 | Mice to Mice FMT | LEfSe | SHA_20_Otu00279 |

*sorted by order of importance

TABLE 12
Relative abundance (%) of Favourable
Fecal Sample from D8, D15 and D16.

| “Favourable” bacteria in Table 1A | D8 | D15 | D16 |
|---|---|---|---|
| Bifidobacterium_bifidum (species) | 0 | 0.31241 | 0.74602 |
| Roseburia_intestinalis (species) | 0 | 3.58995 | 5.9403 |
| Sutterella_wadsworthensis (species) | 0 | 0 | 1.12418 |

TABLE 13
Relative abundance (%) of Unfavourable Fecal Sample from D8, D15 and D16.

| “Unfavourable” bacteria in Table 1B | D8 | D15 | D16 |
|---|---|---|---|
| Blautia_hydrogenotrophica (species) | 0.01821 | 0 | 0 |
| Peptostreptococcaceae_noname_unclassified (species [symbol] | 0.06985 | 0 | 0.00101 |

[symbol]

indicates data missing or illegible when filed

TABLE 14
Characteristics and weight loss of FMT recipients
after receiving FMT from D8, D15 and D16

| Recipient Study | | | | | |
|---|---|---|---|---|---|
| Number | FB001 | FB002 | FB003 | FB004 | FB005 |
| Gender | M | M | F | F | F |
| Stool Donor | D8 | D8 | D8 | D16 | D15 |
| Original weight | 113.4 | 127.1 | 95.5 | 111.6 | 98.3 |
| Weight loss at Week 4 | −2.4% | 0.3% | −0.5% | −2.4% | −1.8% |
| Weight loss at Week 6 | −2.4% | −1.3% | −2.4% | −3.3% | NA |
| Weight loss at Week 8 | −2.8% | 1.0% | −1.9% | −3.8% | −5.5% |

TABLE 15
“Favourable” bacteria in donors for the use of FMT to treat CRE

| | | | | Minimum |
|---|---|---|---|---|
| | | | | relative |
| | | | | abundance in |
| | | | | donor for FMT |
| No. | Study | Analysis | Taxa (species) | (%) |
| 1 | Prospective | Lefse | Ruminococcus_sp_5_1_39BFAA | 12.53 |
| | pilot | | | |
| 2 | Prospective | Lefse | Collinsella_tanakaei | 0.174 |
| | pilot | | | |
| 3 | Prospective | Lefse | Eubacterium_siraeum | 0.0064 |
| | pilot | | | |

TABLE 16
“Favourable” fungi in donors for the use of FMT to treat CRE

| | | | | | Average fold |
|---|---|---|---|---|---|
| | | | | Minimum relative | change in |
| | | | | abundance* in | recipient after |
| No. | Study | Analysis | Taxa (species) | donor for FMT (%) | FMT |
| 1 | Prospective pilot | Lefse | Candida | 0.0955 | 4.3789 |
| | | | duobushaemulonis | | |
| 2 | Prospective pilot | Lefse | Aspergillus arachidicola | 0.0414 | 2.3444 |
| 3 | Prospective pilot | Lefse | Tsuchiyaea wingfieldii | 0.0285 | 4.2174 |
| 4 | Prospective pilot | Lefse | Pseudogymnoascus sp. | 0.0604 | 3.4754 |
| | | | VKM F-4518 | | |
| 5 | Prospective pilot | Lefse | Daedalea quercina | 0.0781 | 1.9931 |
| 6 | Prospective pilot | Lefse | Pseudogymnoascus sp. | 0.0251 | 2.5144 |
| | | | 03VT05 | | |
| 7 | Prospective pilot | Lefse | Xylona heveae | 0.0709 | 2.0832 |
| 8 | Prospective pilot | Lefse | Zymoseptoria brevis | 0.1822 | 2.8792 |
| 9 | Prospective pilot | Lefse | Pisolithus tinctorius | 0.1514 | 2.1794 |
| 10 | Prospective pilot | Lefse | Aspergillus neoniger | 0.1697 | 2.8689 |
| 11 | Prospective pilot | Lefse | Candida intermedia | 0.2633 | 3.0098 |
| 12 | Prospective pilot | Lefse | Ophiocordyceps | 0.1908 | 2.4761 |
| | | | unilateralis | | |
| 13 | Prospective pilot | Lefse | Kwoniella heveanensis | 0.1923 | 1.6540 |
| 14 | Prospective pilot | Lefse | Chaetomium globosum | 0.2576 | 2.1256 |
| 15 | Prospective pilot | Lefse | Cryptococcus | 0.1639 | 5.7038 |
| | | | amylolentus | | |
| 16 | Prospective pilot | Lefse | Stemphylium lycopersici | 0.2031 | 2.3152 |
| 17 | Prospective pilot | Lefse | Leptosphaeria maculans | 0.1375 | 2.5495 |
| 18 | Prospective pilot | Lefse | Aspergillus | 0.1296 | 3.2034 |
| | | | eucalypticola | | |
| 19 | Prospective pilot | Lefse | Aspergillus luchuensis | 0.0325 | 9.7023 |
| 20 | Prospective pilot | Lefse | Aspergillus | 0.2090 | 2.4095 |
| | | | taichungensis | | |
| 21 | Prospective pilot | Lefse | Wallemia mellicola | 0.1534 | 5.2990 |
| 22 | Prospective pilot | Lefse | Ceraceosorus guamensis | 0.0582 | 12.9681 |

1. A method for identifying a suitable donor for FMT, comprising the step of determining level of one or more bacterial species set forth in Table 1a, 4, 6a, 10b, 11, or 15 in a stool sample obtained from a candidate.

2. The method of claim 1, wherein the level of the one or more bacterial species is a percentage relative abundance.

3. The method of claim 1, wherein the level of the one or more bacterial species is greater than 0.1% and the candidate is identified as a suitable donor for FMT.

4. The method of claim 3, further comprising obtaining stool material from the candidate for use in FMT.

5. The method of claim 3, wherein the level of the one or more bacterial species set forth in Table 1b, 6b, or 10a is no greater than 0.01%.

6. The method of claim 5, further comprising obtaining stool material from the candidate for use in FMT.

7. The method of claim 2, wherein the level of the one or more bacterial species set forth in 1b, 6b, or 10a is greater than 0.01% and the candidate is identified as an unsuitable donor for FMT.

8. The method of claim 1, further comprising determining total bacterial load in the stool sample.

9. The method of claim 1, wherein the level of one or more bacterial species set forth in Table 1a, 1b, 4, 6a, 6b, 10a, 10b, 11, or 15 is determined in a first stool sample obtained from a first candidate and in a second stool sample obtained from a second candidate.

10. The method of claim 9, wherein the first candidate has a higher level of the one or more bacterial species set forth in Table 1a, 4, 6a, 10b, 11, or 15 than the second candidate and is deemed to be a more suitable FMT donor than the second candidate.

11. The method of claim 9, wherein the first candidate has a lower level of the one or more bacterial species set forth in Table 1b, 6b, or 10a than the second candidate and is deemed to be a more suitable FMT donor that the second candidate.

12-85. (canceled)

86. A method for weight reduction in a subject, comprising introducing into the subject's gastrointestinal tract an effective amount of one or more of Sutterella wadsworthensis, Roseburia intestinalis, or Mitsuokella multacida.

87. The method of claim 86, comprising introducing into the subject's gastrointestinal tract an effective amount of each of Sutterella wadsworthensis, Roseburia intestinalis, and Mitsuokella multacida.

88. A method for reducing total cholesterol and low-density lipoprotein cholesterol in a subject, comprising introducing into the subject's gastrointestinal tract an effective amount of one or more of Sutterella wadsworthensis, Roseburia intestinalis, or Mitsuokella multacida.

89. The method of claim 88, comprising introducing into the subject's gastrointestinal tract an effective amount of each of Sutterella wadsworthensis, Roseburia intestinalis, and Mitsuokella multacida.
